(19) United States
(12) Patent Application Publication
(10) Pub. No.: US 2023/0338587 A1
(43) Pub. Date: Oct. 26, 2023

(54) METHOD OF TREATING PSMA-EXPRESSING CANCERS

(71) Applicants: ADVANCED ACCELERATOR APPLICATIONS INTERNATIONAL SA, Geneva (CH); Advanced Accelerator Applications USA, Inc., Millburn, NJ (US); Novartis Pharma AG, Basel (CH)

(72) Inventors: Germo GERICKE, Fischingen (DE); Jose-Maria GIMENEZ ANARU, Reinach (CH); Darshan DALAL, Millburn, NJ (US)

(21) Appl. No.: 18/023,217

(22) PCT Filed: Aug. 30, 2021
(86) PCT No.: PCT/EP2021/073900

§ 371 Date: Feb. 24, 2023

(30) Foreign Application Priority Data
Aug. 31, 2020 (EP) 20193733.1

(51) Int. Cl. A61K 51/04; A61K 45/06

(52) U.S. Cl. CPC A61K 51/0497; A61K 51/0402; A61K 45/06

(57) ABSTRACT

The present invention relates to combinations for use and methods of treating cancers that express prostate specific membrane antigen (PSMA). In particular, the invention provides novel therapies based on the combination of a PSMA therapeutic agent, such as radiolabeled Compound I, and immuno-oncology (I-O) therapeutic agents, wherein said I-O therapeutic agents are selected from the group consisting of LAG-3 inhibitors, TIM-3 inhibitors, GITR agonists, TGF-β inhibitors, IL15/IL-15RA, PD-1 inhibitors, PD-L1 inhibitors, and CTLA-4 inhibitors.

### FIELD OF THE INVENTION

The present invention relates to methods of treating prostate cancer and other cancers that express prostate-specific membrane antigen (PSMA), e.g. PSMA expressing cancers such as PSMA over-expressing cancers and cancers that express PSMA in their neovasculature. In particular, the invention provides novel therapies based on the combination of a PSMA therapeutic agent, such as radiolabeled Compound I described herein, and immuno-oncology (I-O) therapeutic agents, wherein said I-O therapeutic agents are selected from LAG-3 inhibitors, TIM-3 inhibitors, GITR agonists, TGF-β inhibitors, IL15/IL-15RA complex, PD-1 inhibitors, PD-L1 inhibitors, and CTLA-4 inhibitors.

### BACKGROUND AND SUMMARY OF THE INVENTION

The prostate is a male reproductive organ and functions to produce and store seminal fluid that provides nutrients and fluids for the survival of sperm introduced into the vagina during reproduction. Like other tissues, the prostate gland may develop either malignant (cancerous) or benign (non-cancerous) tumors. In fact, prostate cancer is one of the most common male cancers in western societies, and is the second leading form of malignancy among American men. Current treatment methods for prostate cancer include hormonal therapy, radiation therapy, surgery, chemotherapy, photodynamic therapy, and combination therapy. However, many of these treatments affect the quality of life of the patient, especially for those men who are diagnosed with prostate cancer over age 50. For example, the use of hormonal drugs is often accompanied by side effects such as osteoporosis and liver damage. Such side effects might be mitigated by the use of treatments that are more selective or specific to the tissue responsible for the disease state, and that avoid non-target tissues like the bones or the liver.

Prostate-specific membrane antigen (PSMA) is a biomarker that is expressed on prostate cancer. PSMA can be over-expressed in malignant prostate tissues when compared to other organs in the human body such as kidney, proximal small intestine, and salivary glands. PSMA is also expressed on the neovasculature within many non-prostate solid tumors, including lung, colon, breast, renal, liver and pancreatic carcinomas, but not on normal vasculature. PSMA is also expressed minimally in brain. PSMA is a type II cell surface membrane-bound glycoprotein with ˜110 kD molecular weight, including an intracellular segment (amino acids 1-18), a transmembrane domain (amino acids 19-43), and an extensive extracellular domain (amino acids 44-750). While the functions of the intracellular segment and the transmembrane domains are currently believed to be insignificant, the extracellular domain is involved in several distinct activities. For example, PSMA plays a role in the central nervous system, where it metabolizes N-acetyl-aspartyl glutamate (NAAG) into glutamic and N-acetyl aspartic acid. PSMA also plays a role in the proximal small intestine where it removes γ-linked glutamate from poly-γ-glutamated folate and α-linked glutamate from peptides and small molecules. However, PSMA's particular function on prostate cancer cells remains unresolved.

PSMA is named largely due to its higher level of expression on prostate cancer cells relative to other tissues; however, its particular function on prostate cancer cells remains unresolved. PSMA expression is highly restricted in normal tissues. It is present in only salivary gland tissue, renal tissue, and on small numbers of cells in the small and large intestine. PSMA is over-expressed in malignant prostate tissues when compared to other organs in the human body such as kidney, proximal small intestine, and salivary glands. Higher PSMA expression is associated with high grade, metastatic and castration resistance disease. Tumor expression of PSMA in prostate cancer is typically 100 to 1,000-fold higher than normal tissues. Unlike many other membrane-bound proteins, PSMA undergoes rapid internalization into the cell in a fashion similar to cell surface bound receptors like vitamin receptors. PSMA is internalized through clathrin-coated pits and subsequently can either recycle to the cell surface or go to lysosomes. Accordingly, diagnostic, imaging, and therapeutic agents can be targeted to PSMA for delivery into PSMA expressing cells, such as prostate cancer cells.

PSMA is also expressed on the neovasculature of cancers other than prostate cancers, such as thyroid cancer, renal clear cell carcinoma, transitional cell carcinoma of the bladder, colonic adenocarcinoma, neuroendocrine carcinoma, glioblastoma multiforme, malignant melanoma, pancreatic duct carcinoma, non-small cell lung carcinoma, soft tissue sarcoma, and breast carcinoma. These cancers represent a large range of different cancers with different histological subtypes, growth rates and cell cycle times. In some cases, the cancers are imbedded within normal tissues having variable radiation tolerances. In addition, hypoxic areas of larger deposits may also lead to radio resistance. These and other factors are known to result in different intrinsic responses to traditional external beam radiation therapy.

Though the activity of the PSMA on the cell surface of prostate cells remains under investigation, it has been recognized that PSMA represents a viable target for the selective and/or specific delivery of biologically active agents or combinations of biologically active agents, including drug compounds, to such prostate cells. One such drug compound is the compound of Formula I

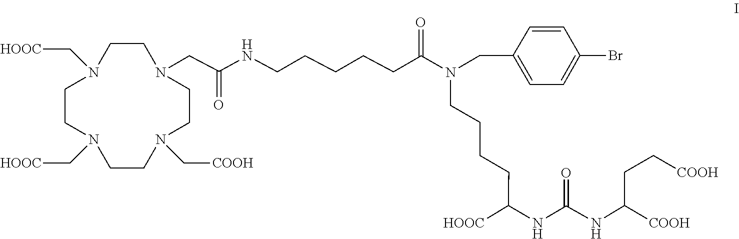 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

Compound I can be described as a small molecule that specifically binds to PSMA (prostate specific membrane antigen) which is expressed on the surface of prostate cancer cells. Compound I can be characterized as composed of a pharmacophore ligand, glutamate-urea-lysine; a chelator, DOTA (able to complex ¹⁷⁷Lu and ²²⁵Ac); and a linker connecting the ligand and the chelator. Without being bound by theory, it is believed that the urea-based pharmacophore ligand allows the agent to bind to, and be internalized by PSMA at the site of disease. It is further believed that the binding of I—Lu or I—Ac can lead to internalization through endocytosis which can provide a sustained retention of the ligand and its bound radioactive cargo within the cancer cell. As used herein, when the term “Compound I” is used in a therapeutic context, it is bound to a radionuclide.

Previous radioligand therapy (RLT) used in the clinic includes ¹³¹I in thyroid cancer, and elements emitting alpha radiation, such as ²²³Radium or ⁸⁹Strontium, for the treatment of bone metastases.

¹⁷⁷Lu has a half-life of 6.7 days. It emits 0.5 MeV energy consisting of negatively charged β particles (electrons) that travel chaotically through tissues for approximately 20-80 cells or 0.5-2 mm and cause predominantly base damage and single strand breaks. At high dose these lesions can interact to convert sublethal damage (SLD) or potentially lethal damage (PLD) to irreparable, lethal damage. ¹⁷⁷Lu also emits 113 Kv and 208 kV radiation which can be used for imaging.

²²⁵Ac has a half-life of 9.9 days, and in contrast emits 8.38 MV energy alpha particles. Only 0.5% of the energy is emitted as 142 Kv photon emissions. The majority of radiation particles are therefore positively charged, and about 8,000 times larger than β particles. Furthermore, the energy from these particles is deposited over relatively short distances (2-3 cells). As a result, there is dense and severe tissue damage in the form of double strand breaks with multiply damaged sites that represent irreparable lethal damage. This is called High Linear Energy Transfer (LET) or densely ionizing ionization and it delivers 3-7× more absorbed dose than β particles. The type of cellular damage inflicted by either isotope (¹⁷⁷Lu or ²²⁵Ac) is expected to be different due to the difference of the characteristics of each warhead. ¹⁷⁷Lu is believed to provide a longer path length of radiation and therefore can be effective in delivering radiation to adjacent cells. The preponderance of single strand breaks, especially in the presence of oxygen, provides the opportunity to repair sub lethal damage (SLD) and or potentially lethal damage (PLD) providing the optimal conditions for normal tissue repair. On the contrary, ²²⁵Ac delivers extremely powerful, high LET radiation, and the potential for repair of normal tissue is much more limited. The radiological biological effectiveness of alpha radiation is at least 5 times that of beta irradiation and for administered doses the relative biological effectiveness (RBE) has to be taken into account. With ²²⁵Ac therapy, the type of DNA damage inflicted does not require the presence of oxygen so it will also be more effective in hypoxic tumor regions. A possible disadvantage of ²²⁵Ac therapy is that the short path length can lead to large amounts of damaging radiation deposited only within a short distance of 2-4 cells.

Combinations of therapeutic agents and methods of treating PSMA-expressing cancers based on the combination of a PSMA therapeutic agent and immuno-oncology (I-O) therapeutic agents are described herein.

Immuno-oncology (I-O) therapeutic agents can defeat the established tolerance toward the cancer and recover an effective cancer-specific immune response.

The tumor-cell internal radiation provided by radiolabeled Compound I described herein can cause damage to the tumor and the release of tumor antigens, thus making the tumor more visible to the immune system. I-O therapy provides immune checkpoint blockade and therefore improves the immune anti-tumor T-cell response. In this way the I-O therapy may enhance the effect of the internal radiation by radiolabeled Compound I in a synergistic way.

The present invention provides novel combinations comprising a PSMA therapeutic agent and one or more immuno-oncology (I-O) therapeutic agent(s) for use in treating a PSMA expressing cancer in a subject, wherein said I-O therapeutic agent(s) is (are) selected from the group consisting of LAG-3 inhibitors, TIM-3 inhibitors, GITR angonists, TGF-β inhibitors, IL15/IL-15RA complexes, PD-1 inhibitors, PD-L1 inhibitors, and CTLA-4 inhibitors, wherein said PD-1 inhibitors are selected from the group consisting of Spartalizumab, Pembrolizumab, Pidilizumab, Durvalomab, Atezolizumab, Avelumab, Nivolumab, MK-3475, MPDL3280A, MEDI4736, ipilimumab, tremelimumab, MEDI0680, REGN2810, TSR-042, PF-06801591, BGB-A317, BGB-108, INCSHR1210, and AMP-224, and wherein the PSMA therapeutic agent is radiolabeled Compound I.

The combinations or methods according to the present invention may comprise one or more further anti-cancer agent(s).

In the combinations or methods according to the present invention, the LAG-3 inhibitor may be selected from LAG525, BMS-986016, or TSR-033.

In the combinations or methods according to the present invention, the TIM-3 inhibitor may be MBG453 or TSR-022.

In the combinations or methods according to the present invention, the GITR agonist may be selected from GWN323, BMS-986156, MK-4166, MK-1248, TRX518, INCAGN1876, AMG 228, or INBRX-110.

In the combinations or methods according to the present invention, the TGF-β inhibitor may be XOMA 089 or fresolimumab.

In the combinations or methods according to the present invention, the IL-15/IL-15RA complex may be selected from NIZ985, ATL-803 or CYP0150.

Further anti-cancer agents according to the present invention may be selected from octreotide, lanreotide, vaproreotide, pasireotide, satoreotide, everolimus, temozolomide, telotristat, sunitinib, sulfatinib, ribociclib, entinostat, and pazopanib.

The present invention provides the following exemplary embodiments:

1. A Combination Comprising a Compound of Formula I (Compound I)

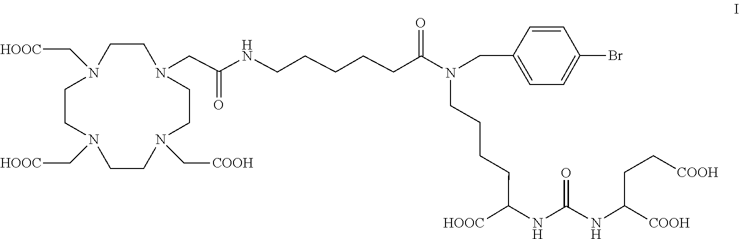 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

wherein Compound I is radiolabeled and one or more immuno-oncology (I-O) therapeutic agent(s) for use in treating a PSMA expressing cancer in a subject, wherein said I-O therapeutic agent(s) is (are) selected from LAG-3 inhibitors, TIM-3 inhibitors, GITR angonists, TGF-β inhibitors, IL15/IL-15RA complexes, PD-1 inhibitors, PD-L1 inhibitors, and CTLA-4 inhibitors, wherein said PD-1 inhibitors are selected from Spartalizumab, Pembrolizumab, Pidilizumab, Durvalomab, Atezolizumab, Avelumab, Nivolumab, MK-3475, MPDL3280A, MEDI4736, ipilimumab, tremelimumab, MEDI0680, REGN2810, TSR-042, PF-06801591, BGB-A317, BGB-108, INCSHR1210, and AMP-224.

2. A Method of Treating a PSMA Expressing Cancer in a Subject, Comprising Administering to the Subject a Combination of a Compound of Formula I (Compound I)

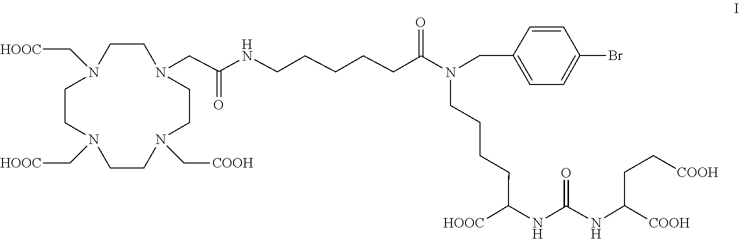 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

wherein Compound I is radiolabeled and one or more immuno-oncology (I-O) therapeutic agent(s), wherein said I-O therapeutic agent(s) is(are) selected from an LAG-3 inhibitor, a TIM-3 inhibitor, a GITR angonists, a TGF-β inhibitor, an IL15/IL-15RA complex, and a PD-1 inhibitor, wherein said PD-1 inhibitor is selected from Spartalizumab, Pembrolizumab, Pidilizumab, Durvalomab, Atezolizumab, Avelumab, Nivolumab, MK-3475, MPDL3280A, MEDI4736, ipilimumab, tremelimumab, MEDI0680, REGN2810, TSR-042, PF-06801591, BGB-A317, BGB-108, INCSHR1210, and AMP-224.

3. The combination of embodiment 1, or the method of embodiment 2, wherein the radiolabeled Compound I and the I-O therapeutic agent(s) are in separate compositions and are administered to the subject separately.

4. The combination of any one of embodiments 1 or 3, or the method of any one of embodiments 2 or 3, wherein the LAG-3 inhibitor is chosen from LAG525, BMS-986016, or TSR-033.

5. The combination of any one of embodiments 1 or 3 to 4, or the method of any one of embodiments 2 to 4, wherein the TIM-3 inhibitor is MBG453 or TSR-022.

6. The combination of any one of embodiments 1 or 3 to 5, or the method of any one of embodiments 2 to 5, wherein the GITR agonist is chosen from GWN323, BMS-986156, MK-4166, MK-1248, TRX518, INCAGN1876, AMG 228, or INBRX-110.

7. The combination of any one of embodiments 1 or 3 to 6, or the method of any one of embodiments 2 to 6, wherein the TGF-β inhibitor is XOMA 089 or fresolimumab.

8. The combination of any one of embodiments 1 or 3 to 7, or the method of any one of embodiments 2 to 7, wherein the IL-15/IL-15RA complex is chosen from NIZ985, ATL-803 or CYP0150.

9. The combination of any one of embodiments 1 or 3 to 8, or the method of any one of embodiments 2 to 8, comprising one or more further anti-cancer agent(s).

10. The combination or the method of embodiment 9, wherein the further anti-cancer agent(s) is (are) selected from octreotide, lanreotide, vaproreotide, pasireotide, satoreotide, everolimus, temozolomide, telotristat, sunitinib, sulfatinib, ribociclib, entinostat, and pazopanib.

11. The combination of any one of embodiments 1 or 3 to 10, or the method of any one of embodiments 2 to 10, wherein the PSMA expressing cancer is a prostate cancer.

12 The combination of any one of embodiments 1 or 3 to 10 or the method of any one of embodiments 2 to 10, wherein the PSMA-expressing cancer is selected from thyroid cancer, renal clear cell carcinoma, transitional cell carcinoma of the bladder, colonic adenocarcinoma, neuroendocrine carcinoma, glioblastoma multiforme, malignant melanoma, pancreatic duct carcinoma, non-small cell lung carcinoma, soft tissue sarcoma, and breast carcinoma.

13. The combination or the method of embodiment 12 wherein the PSMA is expressed on the neovasculature of the cancer.

14. The combination of any one of embodiments 1 or 3 to 13 or the method of any one of embodiments 2 to 13 wherein Compound I binds a radionuclide selected from ¹⁷⁷Lu and ²²⁵Ac.

15. The combination or the method of embodiment 14 wherein the Compound I is bound to ¹⁷⁷Lu.

16. The combination or the method of embodiment 14 wherein the Compound I is bound to ²²⁵Ac.

17. The combination or the method of embodiment 14 wherein both the Compound I bound to ¹⁷⁷Lu and the Compound I bound to ²²⁵Ac are administered to the subject.

18. The combination of any one of embodiments 1 or 3 to 16 or the method of any one of embodiments 2 to 17 wherein a PD-1 inhibitor is administered and the PD-1 inhibitor is not Pembrolizumab.

19. The combination or the method of embodiment 15 or 17 wherein the amount of the Compound I bound to ¹⁷⁷Lu that is administered is from about 2 GBq to about 13 GBq.

20. The combination or the method of embodiment 15 or 17 wherein the amount of the Compound I bound to ¹⁷⁷Lu that is administered is from about 4 GBq to about 11 GBq.

21. The combination or the method of embodiment 15 or 17 wherein the amount of the Compound I bound to ¹⁷⁷Lu that is administered is from about 5 GBq to about 10 GBq.

22. The combination or the method of embodiment 15 or 17 wherein the amount of the Compound I bound to ¹⁷⁷Lu that is administered is from about 6 GBq to about 9 GBq.

23. The combination or the method of embodiment 15 or 17 wherein the amount of the Compound I bound to ¹⁷⁷Lu that is administered is from about 6.5 GBq to about 8.5 GBq.

24. The combination or the method of embodiment 15 or 17 wherein the amount of the Compound I bound to ¹⁷⁷Lu that is administered is from about 7 GBq to about 8 GBq.

25. The combination or the method of embodiment 16 or 17 wherein the amount of the Compound I bound to ²²⁵Ac that is administered is from about 1 MBq to about 6 MBq.

26. The combination or the method of embodiment 16 or 17 wherein the amount of the Compound I bound to ²²⁵Ac that is administered is from about 1 MBq to about 5 MBq.

27. The combination or the method of embodiment 16 or 17 wherein the amount of the Compound I bound to ²²⁵Ac that is administered is from about 1 MBq to about 4 MBq.

28. The combination or the method of embodiment 16 or 17 wherein the amount of the Compound I bound to ²²⁵Ac that is administered is from about 1 MBq to about 3 MBq.

29. The combination or the method of embodiment 16 or 17 wherein the amount of the Compound I bound to ²²⁵Ac that is administered is or from about 2 MBq to about 3 MBq.

30. The combination or the method of embodiment 16 or 17 wherein the amount of the Compound I bound to ²²⁵Ac that is administered is about 2 MBq.

31. The combination of any one of embodiments 1, 3 to 16, or 18 to 30 or the method of any one of embodiments 2 to 30 wherein the I-O therapeutic agent is nivolumab.

32. The combination of any one of embodiments 1, 3 to 16, or 18 to 30 or the method of any one of embodiments 2 to 30 wherein the I-O therapeutic agent is ipilimumab.

33. The combination of any one of embodiments 1, 3 to 16, or 18 to 30 or the method of any one of embodiments 2 to 30 wherein the I-O therapeutic agent is tremelimumab.

In some embodiments, the combinations described herein may provide a beneficial anti-cancer effect, e.g., an enhanced anti-cancer effect, reduced toxicity, and/or reduced side effects. For example, the PSMA therapeutic agent, such as radiolabeled Compound I, and a second therapeutic agent, e.g., the one or more additional therapeutic agents, or all, such as the I-O therapeutic agent(s), can be administered at a lower dosage than would be required to achieve the same therapeutic effect compared to a monotherapy dose. Thus, compositions and methods for treating proliferative disorders, including cancer, using the aforesaid combination therapies are disclosed.

In some embodiments, a method of treating a subject, e.g., a subject having a cancer described herein, with a combination described herein, comprises administration of a combination as part of a therapeutic regimen. In an embodiment, a therapeutic regimen comprises one or more, e.g., two, three, or four combinations described herein. In some embodiments, the therapeutic regimen is administered to the subject in at least one phase, and optionally two phases, e.g., a first phase and a second phase. In some embodiments, the first phase comprises a dose escalation phase. In some embodiments, the first phase comprises one or more dose escalation phases, e.g., a first, second, or third dose escalation phase. In some embodiments, the dose escalation phase comprises administration of a combination comprising two, three, four, or more therapeutic agents, e.g., as described herein. In some embodiments, the second phase comprises a dose expansion phase. In some embodiments, the dose expansion phase comprises administration of a combination comprising two, three, four, or more therapeutic agents, e.g., as described herein. In some embodiments, the dose expansion phase comprises the same two, three, four, or more therapeutic agents as the dose escalation phase.

In some embodiments, the first dose escalation phase comprises administration of a combination comprising a PSMA therapeutic agent, such as radiolabeled Compound I, and one or more additional therapeutic agents, such as the I-O therapeutic agent(s) described herein, wherein a maximum tolerated dose (MTD) or recommended dose for expansion (RDE) for one or both of the therapeutic agents is determined. In some embodiments, prior to the first dose escalation phase, the subject can be administered with one of the therapeutic agents administered in the first dose escalation phase as a single agent.

In some embodiments, the second dose escalation phase comprises administration of a combination comprising a PSMA therapeutic agent, such as radiolabeled Compound I, and one or more additional therapeutic agents, e.g., such as two of the I-O therapeutic agent(s) described herein, wherein a maximum tolerated dose (MTD) or recommended dose for expansion (RDE) for one, two, or all of the therapeutic agents is determined. In some embodiments, the second dose escalation phase starts after the first dose escalation phase ends. In some embodiments, the second dose escalation phase comprises administration of one or more of the therapeutic agents administered in the first dose escalation phase. In some embodiments, the second dose escalation phase is performed without performing the first dose escalation phase.

In some embodiments, the third dose escalation phase comprises administration of a combination comprising a PSMA therapeutic agent, such as radiolabeled Compound I, and one or more additional therapeutic agents, e.g., such as three of the I-O therapeutic agent(s) described herein, wherein a maximum tolerated dose (MTD) or recommended dose for expansion (RDE) of one, two, three, or all of the therapeutic agents is determined. In some embodiments, the third dose escalation phase starts after the first or second dose escalation phase ends. In some embodiments, the third dose escalation phase comprises administration of one or more (e.g., all) of therapeutic agents administered in the second dose escalation phase. In some embodiments, the third dose escalation phase comprises administration of one or more of the therapeutic agents administered in the first dose escalation phase. In some embodiments, the third dose escalation phase is performed without performing the first, second, or both dose escalation phases.

In some embodiments, the dose expansion phase starts after the first, second or third dose escalation phase ends. In some embodiments, the dose expansion phase comprises administration of a combination administered in the dose escalation phase, e.g., the first, second, or third dose escalation phase. In an embodiment, a biopsy is obtained from a subject in the dose expansion phase. In an embodiment, the subject is treated for prostate cancer.

Without wishing to be bound by theory, it is believed that in some embodiments, a therapeutic regimen comprising a dose escalation phase and a dose expansion phase allows for entry of new agents or regiments for combination, rapid generation of combinations, and/or assessment of safety and activity of tolerable combinations.

### DETAILED DESCRIPTION

### Definitions

As used herein, the term “alkyl” includes a chain of carbon atoms, which is optionally branched and contains from 1 to 4 carbon atoms, and the like, and may be referred to as “lower alkyl.” Illustrative alkyl groups include, but are not limited to, methyl, ethyl, n-propyl, isopropyl, n-butyl, isobutyl, sec-butyl, and tert-butyl.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise. The word “or”, however, in reference to claims that are in a multiple dependent format, means “or” and is not interchangeable with “and”.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The term “therapeutic agent(s)” encompasses all therapeutic agents described in this application unless otherwise specified.

By “combination” or “in combination with,” it is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapies can be administered in any order. In general, each therapeutic agent will be administered at a dose and/or using a regimen determined for that therapeutic agent. It will further be appreciated that the therapeutic agents utilized in this combination may be administered together in a single composition or administered separately in different compositions. In some embodiments, the therapeutic agents utilized in combination can be utilized at levels that do not exceed the levels at which they are typically utilized individually. In some embodiments, the levels of the therapeutic agents utilized in combination can be lower than those utilized individually.

In some embodiments, the therapeutic agent is administered at a therapeutic dose or a dose that is lower-than the therapeutic dose when administered individually. In certain embodiments, the concentration of the second therapeutic agent administered when administered in a combination is lower than the dose that would cause therapeutic efficacy when the second therapeutic agent is administered individually. In certain embodiments, the concentration of the first therapeutic agent when administered in a combination is lower than the dose that would cause therapeutic efficacy when the first therapeutic agent is administered individually. In certain embodiments, in a combination therapy, the concentration of the second therapeutic agent that is required to achieve inhibition, e.g., growth inhibition, is lower than the therapeutic dose of the second therapeutic agent as a monotherapy, e.g., 10-20%, 20-30%, 30-40%, 40-50%, 50-60%, 60-70%, 70-80%, or 80-90% lower. In certain embodiments, in a combination therapy, the concentration of the first therapeutic agent that is required to achieve inhibition, e.g., growth inhibition, is lower than the therapeutic dose of the first therapeutic agent as a monotherapy, e.g., at least or about 10-20%, 20-30%, 30-40%, 40-50%, 50-60%, 60-70%, 70-80%, or 80-90% lower.

The term “inhibition,” “inhibitor,” or “antagonist” includes a reduction in a certain parameter, e.g., an activity, of a given molecule, e.g., an immune checkpoint inhibitor. For example, inhibition of an activity, e.g., an activity of a given molecule, e.g., an inhibitory molecule, of at least or about 5%10%, 20%, 30%, 40%, 50%, 60%, 70%, 80%, 90%, 95%, or more is included by this term. Thus, inhibition need not be 100%.

A “fusion protein” and a “fusion polypeptide” refer to a protein or polypeptide having at least two portions covalently linked together, where each of the portions is a polypeptide having a different property. The property may be a biological property, such as activity in vitro or in vivo. The property can also be a simple chemical or physical property, such as binding to a target molecule, catalysis of a reaction, etc. The two portions can be linked directly by a single peptide bond or through a peptide linker, but are in reading frame with each other.

The term “activation,” “activator,” or “agonist” includes an increase in a certain parameter, e.g., an activity, of a given molecule, e.g., a costimulatory molecule. For example, increase of an activity, e.g., a costimulatory activity, of at least or about 5%, 10%, 15%, 25%, 30%, 35%, 40%, 45%, 50%, 55%, 60%, 65%, 70%, 75%, 80%, 85%, 90%, 95%, or more is included by this term.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the therapeutic agents described herein (e.g., peptides, polynucleotides, cells, small molecules, and antibodies to prevent the occurrence of cancer in the first place.

The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The term “cancer” refers to a disease characterized by the rapid and uncontrolled growth of aberrant cells, but can include benign cancers. In various embodiments, cancer cells can spread locally or through the bloodstream and lymphatic system to other parts of the body. In some aspects, the cancer is a PSMA expressing cancer. In some embodiments, the PSMA can be expressed on the neovasculature of the cancer. Examples of various cancers are described herein include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, renal clear cell carcinoma, transitional cell carcinoma of the bladder, colonic adenocarcinoma, neuroendocrine carcinoma, glioblastoma multiforme, malignant melanoma, pancreatic duct carcinoma, non-small cell lung carcinoma, soft tissue sarcoma, and the like. In some embodiments, the cancer is selected from the group consisting of a glioma, a carcinoma, a sarcoma, a lymphoma, a melanoma, a mesothelioma, a nasopharyngeal carcinoma, a leukemia, an adenocarcinoma, and a myeloma. Other exemplary cancers include small cell lung cancer, bone cancer, cancer of the head or neck, hepatocellular carcinoma, cutaneous or intraocular melanoma, uterine cancer, stomach cancer, colon cancer, carcinoma of the fallopian tubes, carcinoma of the endometrium, carcinoma of the cervix, carcinoma of the vagina, carcinoma of the vulva, Hodgkin's Disease, gastric and esophago-gastric cancers, cancer of the endocrine system, cancer of the parathyroid gland, cancer of the adrenal gland, cancer of the urethra, cancer of the penis, cancer of the ureter, neoplasms of the central nervous system (CNS), primary CNS lymphoma, spinal axis tumors, brain stem glioma, pituitary adenoma, inflammatory myofibroblastic tumors, and combinations thereof.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors and benign cancers. The term “cancer” as used herein includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

As used herein, the terms “treat,” “treatment” and “treating” refer to the reduction or amelioration of the progression, severity and/or duration of a disorder, e.g., a proliferative disorder, such as cancer, or the amelioration of one or more symptoms (e.g., one or more discernible symptoms) of the disorder resulting from the administration of one or more therapies or therapeutic agents. In specific embodiments, the terms “treat,” “treatment” and “treating” refer to the amelioration of at least one measurable physical parameter of a proliferative disorder, such as a cancer, for example, growth of a tumor, not necessarily discernible by the patient. In other embodiments the terms “treat”, “treatment” and “treating” refer to the inhibition of the progression of a proliferative disorder, such as a cancer, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

In many of the embodiments described herein, the therapeutic agent administered as described herein, in a combination with a PSMA therapeutic agent, such as radiolabeled Compound I, can be an antibody or another polypeptide, or a nucleic acid encoding such a polypeptide. In some embodiments, the therapeutic agents described herein for use in the combinations and methods, with a PSMA therapeutic agent, such as radiolabeled Compound I, encompass polypeptides and/or nucleic acids having the sequences specified herein, or sequences substantially identical or similar thereto, e.g., sequences at least 85%, 90%, 95%, 96%, 97%, 98%, 99% identical or higher to the sequence specified. In the context of an amino acid sequence, the term “substantially identical” is used herein to refer to a first amino acid sequence that contains a sufficient or minimum number of amino acid residues that are i) identical to, or ii) conservative substitutions of aligned amino acid residues in a second amino acid sequence, such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., a sequence provided herein, are substantially similar to the amino acid sequence provided herein.

In the context of a nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein, are substantially identical to the nucleic acid sequence provided herein.

The term “functional variant” refers to polypeptides that have a substantially identical amino acid sequence to the naturally-occurring sequence or a sequence provided herein, are encoded by a substantially identical nucleotide sequence, and are capable of having one or more activities of the naturally-occurring sequence or a sequence provided herein.

In various embodiments, calculations of homology or sequence identity or similarity between sequences (the terms are used interchangeably herein) can be performed as follows.

To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes). In various embodiments, the length of a reference sequence aligned for comparison purposes is at least 30%, at least 40%, at least 50% or 60%, or at least 70%, 80%, 90%, or 100% of the length of the reference sequence. The amino acid residues or nucleotides at corresponding amino acid positions or nucleotide positions can then be compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account, for example, the number of gaps, and the length of each gap, which need to be introduced for optimal alignment of the two sequences.

In one embodiment, the comparison of sequences and determination of percent identity between two sequences can be accomplished using a mathematical algorithm. In one embodiment, the percent identity between two amino acid sequences is determined using the Needleman and Wunsch ((1970) J. Mol. Biol. 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. In one aspect, a set of parameters that can be used is a Blossum 62 scoring matrix with a gap penalty of 12, a gap extend penalty of 4, and a frameshift gap penalty of 5.

In one illustrative embodiment, the percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

In one aspect, the nucleic acid and amino acid sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. In one embodiment, BLAST nucleotide searches can be performed with the NBLAST program, score=100, wordlength=12 to obtain nucleotide sequences homologous to a nucleic acid molecule described herein. In one aspect, BLAST amino acid sequence searches can be performed with the XBLAST program, score=50, wordlength=3 to obtain amino acid sequences homologous to amino acid sequences described herein. In one illustrative aspect, to obtain gapped alignments for comparison purposes, Gapped BLAST can be utilized as described in Altschul et al., (1997) Nucleic Acids Res. 25:3389-3402. In another aspect, when utilizing BLAST and Gapped BLAST programs, the default parameters of the respective programs (e.g., XBLAST and NBLAST) can be used. See www.ncbi.nlm.nih.gov.

As used herein, the term “hybridizes under low stringency, medium stringency, high stringency, or very high stringency conditions” describes conditions for hybridization and washing. Exemplary guidance for performing hybridization reactions can be found in Current Protocols in Molecular Biology, John Wiley & Sons, N.Y. (1989), 6.3.1-6.3.6, which is incorporated herein by reference. Aqueous and nonaqueous methods are described in that reference and either can be used. Exemplary specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and 4) very high stringency hybridization conditions are 0.5 M sodium phosphate, 7% SDS at 65° C., followed by one or more washes at 0.2×SSC, 1% SDS at 65° C. In one aspect, therapeutic agents for use in the combinations and methods described herein may have additional conservative or non-essential amino acid substitutions relative to the therapeutic agents described herein, which do not have a substantial effect on their functions.

The term “amino acid” is intended to embrace all molecules, whether natural or synthetic, which include both an amino functionality and an acid functionality and are capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, by disulfide bond formation, glycosylation, lipidation, acetylation, phosphorylation, or any other manipulation, such as conjugation with a labeling component. In various embodiments, the polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. In several embodiments, the polynucleotide may be either single-stranded or double-stranded, and if single-stranded may be the coding strand or non-coding (antisense) strand. In various embodiments, a polynucleotide may comprise modified nucleotides, such as methylated nucleotides and nucleotide analogs, the sequence of nucleotides may be interrupted by non-nucleotide components, and/or the polynucleotide may be further modified after polymerization, such as by conjugation with a labeling component. In various embodiments, the nucleic acid may be a recombinant polynucleotide, or a polynucleotide of genomic, cDNA, semisynthetic, or synthetic origin which either does not occur in nature or is linked to another polynucleotide in a nonnatural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

Various aspects of the invention are described in further detail below. Additional definitions are set out throughout the specification.

### PSMA Therapeutic Agents

The combinations described herein comprise a PSMA therapeutic agent, such as radiolabeled Compound I, and one or more additional therapeutic agent as described below, which can be administered to a patient to treat a cancer, particularly PSMA expressing cancer. The additional therapeutic agent(s) can be any of the therapeutic agents described herein, including one or more of the I-O agents described above. In one embodiment, the PSMA therapeutic agent is a compound of Formula I wherein the compound is radiolabeled

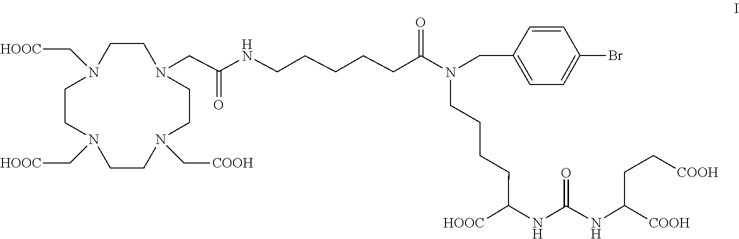 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]

In one embodiment of the combinations for use or the methods described herein, wherein the PSMA therapeutic agent is radiolabeled Compound I, radiolabeled Compound I can bind a radionuclide selected from ¹⁷⁷Lu and ²²⁵Ac. In one aspect, radiolabeled Compound I, bound to ¹⁷⁷Lu is administered. In another embodiment, radiolabeled Compound I bound to ²²⁵Ac is administered. In yet another embodiment, radiolabeled Compound I bound to ¹⁷⁷Lu, and radiolabeled Compound I bound to ²²⁵Ac, are both administered. The PSMA therapeutic agent, such as radiolabeled Compound I, can be administered in a parenteral dosage form. In some embodiments, the parenteral dosage form is selected from the group consisting of intradermal, subcutaneous, intramuscular, intraperitoneal, intravenous, and intrathecal.

In various embodiments, where radiolabeled Compound I bound to ¹⁷⁷Lu is administered, the amount administered is from about 2 GBq to about 13 GBq, from about 4 GBq to about 11 GBq, from about 5 GBq to about 10 GBq, from about 6 GBq to about 9 GBq, from about 6.5 GBq to about 8.5 GBq, or from about 7 GBq to about 8 GBq. In various embodiments, the amount administered is about 2 GBq, about 3 GBq, about 4 GBq, about 5 GBq, about 6 GBq, about 7 GBq, about 8 GBq, about 9 GBq, about 10 GBq, or about 7.4 GBq. In some embodiments, the total dose of radiolabeled Compound I bound to ¹⁷⁷Lu ranges from about 15 GBq to about 200 GBq, from about 25 GBq to about 185 GBq, from about 35 GBq to about 150 GBq, from about 40 GBq to about 100 GBq, from about 40 GBq to about 90 GBq, from about 40 GBq to about 80 GBq, from about 40 GBq to about 70 GBq, from about 40 GBq to about 60 GBq, from about 40 GBq to about 50 GBq, from about 42 GBq to about 58 GBq. In other embodiments, the total dose of radiolabeled Compound I bound to ¹⁷⁷Lu is about 20 GBq, about 30 GBq, about 40 GBq, about 41 GBq, about 42 GBq, about 43 GBq, about 44 GBq, about 45 GBq, about 46 GBq, about 47 GBq, about 48 GBq, about 49 GBq, about 50 GBq, about 60 GBq, or about 70 GBq. In some embodiments, the maximum duration of treatment of a subject is about 19 to about 23 months.

In some embodiments, where radiolabeled Compound I bound to ²²⁵Ac is administered, the amount administered is from about 1 MBq to about 20 MBq, from about 4 MBq to about 14 MBq, from about 5 MBq to about 10 MBq, from about 6 MBq to about 8 MBq, from about 1 MBq to about 10 MBq, from about 1 MBq to about 9 MBq, from about 1 MBq to about 8 MBq, from about 1 MBq to about 7 MBq, from about 1 MBq to about 6 MBq, from about 1 MBq to about 5 MBq, from about 1 MBq to about 4 MBq, from about 1 MBq to about 3 MBq, or from about 2 MBq to about 3 MBq. In other embodiments, the amount administered is about 1 MBq, about 2 MBq, about 2.5 MBq, about 3 MBq, about 4 MBq, about 5 MBq, about 6 MBq, about 7 MBq, about 8 MBq, about 9 MBq, or about 10 MBq.

In other aspects, the combinations and methods described herein further comprise imaging PSMA expression by the cancer. In some embodiments, the step of imaging occurs before the step of administering the PSMA therapeutic agent, such as radiolabeled Compound I. In other embodiments, the step of imaging occurs after the step of administering the PSMA therapeutic agent, such as radiolabeled Compound I. In various embodiments, the imaging method is selected from the group consisting of SPECT imaging, positron-emission tomography imaging, IHC, and FISH. In one embodiment, the imaging is performed by SPECT imaging.

### Additional Therapeutic Agent

The combination according to the invention comprises a PSMA therapeutic agent as described above, such as radiolabeled Compound I and one or more additional therapeutic agent, such as immuno-oncology (I-O) therapeutic agents, as described below.

### Antibody Molecules

In one embodiment, a combination described herein comprises an additional therapeutic agent which is an antibody molecule along with a PSMA therapeutic agent, such as radiolabeled Compound I, or a method described herein uses such a therapeutic agent which is an antibody molecule, along with a PSMA therapeutic agent, such as radiolabeled Compound I.

As used herein, the term “antibody molecule” refers to a protein comprising at least one immunoglobulin variable domain sequence. The term antibody molecule includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. In one aspect, functional antibody fragments retain the ability to selectively bind with their respective antigen or receptor. In various embodiments, antibodies and antibody fragments can be from any class of antibodies including, but not limited to, IgG, IgA, IgM, IgD, and IgE, and from any subclass (e.g., IgG1, IgG2, IgG3, and IgG4) of antibodies, and can be monoclonal or polyclonal, human, humanized, CDR-grafted, or an in vitro generated antibody. In other aspects, the antibody can have a heavy chain constant region chosen from, e.g., IgG1, IgG2, IgG3, or IgG4. In another embodiment, the antibody can have a light chain chosen from, e.g., kappa or lambda.

Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. In one aspect, these antibody fragments can be obtained using conventional techniques known to those with skill in the art, and the fragments can be screened for utility in the same manner as are intact antibodies.

The term “antibody” includes intact molecules as well as functional fragments thereof. In various embodiments, constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

In one aspect, antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any known in the art, or any future single domain antibodies. In various embodiments, single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity, this variable domain derived from a heavy chain antibody naturally devoid of light chain is described herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to the antigen, or an epitope thereof. With respect to proteins (or protein mimetics), the antigen-binding site typically includes one or more loops (of at least four amino acids or amino acid mimics) that form an interface that binds to the antigen. Typically, the antigen-binding site of an antibody molecule includes at least one or two CDRs and/or hypervariable loops, or more typically at least three, four, five or six CDRs and/or hypervariable loops.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

In various embodiments, the antibody molecule can be a polyclonal or a monoclonal antibody. In other embodiments, the antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. In one aspect, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest can be used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

In one aspect, an antibody can be one in which the variable region, or a portion thereof, e.g., the CDRs, are generated in a non-human organism, e.g., a rat or mouse. In another aspect, chimeric, CDR-grafted, and humanized antibodies can be used. In another embodiment, antibodies generated in a non-human organism, e.g., a rat or mouse, and then modified, e.g., in the variable framework or constant region, to decrease antigenicity in a human can be used.

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

In one embodiment, a humanized or CDR-grafted antibody can have at least one or two but generally all three recipient CDRs (of heavy and or light immuoglobulin chains) replaced with a donor CDR. In various aspects, the antibody may be replaced with at least a portion of a non-human CDR or only some of the CDRs may be replaced with non-human CDRs. In one illustrative embodiment, the number of CDRs required for binding of the humanized antibody to the antigen can be replaced. In one aspect, the donor can be a rodent antibody, e.g., a rat or mouse antibody, and the recipient can be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). In one exemplary embodiment, the acceptor framework is a naturally-occurring (e.g., a human) framework or a consensus framework, or a sequence about 85% or higher, or with about 90%, 95%, 96%, 97%, 98%, 99% or higher identity thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated herein by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare humanized antibodies used in the combinations and methods described herein (UK Patent Application GB 2188638A, filed on Mar. 26, 1987; Winter U.S. Pat. No. 5,225,539), the contents of which is expressly incorporated by reference herein.

In additional aspects, humanized antibodies in which specific amino acids have been substituted, deleted or added can be used. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference herein. Other techniques for humanizing antibodies are described in Padlan et al. EP 519596 A1, published on Dec. 23, 1992.

In one embodiment, the antibody molecule can be a single chain antibody. In one aspect, a single-chain antibody (scFV) may be engineered (see, for example, Colcher, D. et al. (1999) Ann N Y Acad Sci 880:263-80; and Reiter, Y. (1996) Clin Cancer Res 2:245-52). In another aspect, the single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; or chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. In another aspect, the constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment, the antibody has effector function and can fix complement. In other embodiments, the antibody does not recruit effector cells or fix complement. In another embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

Methods for altering an antibody constant region are known in the art. In one embodiment, antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar types of alterations could be used which if applied to the murine, or other species immunoglobulin would reduce or eliminate these functions.

In one embodiment, an antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules for use a therapeutic agents along with a PSMA therapeutic agent, such as radiolabeled Compound I, described herein may include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

In one aspect, one type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

In various aspects, an antibody molecule may be conjugated to another molecular entity, typically a label or a therapeutic (e.g., a cytotoxic or cytostatic) agent or moiety. Radioactive isotopes can be used in therapeutic applications. Radioactive isotopes that can be coupled to the antibodies include, but are not limited to α-, β-, or γ-emitters, or β- and γ-emitters. Such radioactive isotopes include, but are not limited to iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹ mTc), phosphorus (³²P), rhodium (¹⁸⁸Rh), sulfur (35S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵I), indium (¹¹¹In), technetium (⁹⁹mTc), phosphorus (³²P), carbon (¹⁴C), and tritium (³H), or one or more of the therapeutic isotopes listed above.

In one aspect, radiolabeled antibody molecules and methods of labeling the same are provided. In one embodiment, a method of labeling an antibody molecule is disclosed. The method includes contacting an antibody molecule, with a chelating agent, to thereby produce a conjugated antibody. The conjugated antibody can be radiolabeled with a radioisotope, e.g., 111Indium, 90Yttrium and 177Lutetium, to thereby produce a labeled antibody molecule.

In one embodiment, as is discussed above, the antibody molecule can be conjugated to a therapeutic agent. Therapeutically active radioisotopes have already been mentioned. Examples of other therapeutic agents include taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846, 545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

### Multispecific Antibody Molecules

In an embodiment, an antibody molecule for use with PSMA therapeutic agents, such as radiolabeled Compound I, as described herein is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap. In an embodiment, the first and second epitopes do not overlap. In an embodiment, the first and second epitopes are on different antigens, e.g., the different proteins (or different subunits of a multimeric protein). In an embodiment, a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule.

In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

Protocols for generating bispecific or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhydryl reactive groups, as described in, e.g., U.S. Pat. No. 5,273,743; biosynthetic binding proteins, e.g., pair of scFvs cross-linked through C-terminal tails through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH—CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also disclosed creating bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002/004587A1, US2002/076406A1, US2002/103345A1, US2003/207346A1, US2003/211078A1, US2004/219643A1, US2004/220388A1, US2004/242847A1, US2005/003403A1, US2005/004352A1, US2005/069552A1, US2005/079170A1, US2005/100543A1, US2005/136049A1, US2005/136051A1, US2005/163782A1, US2005/266425A1, US2006/083747A1, US2006/120960A1, US2006/204493A1, US2006/263367A1, US2007/004909A1, US2007/087381A1, US2007/128150A1, US2007/141049A1, US2007/154901A1, US2007/274985A1, US2008/050370A1, US2008/069820A1, US2008/152645A1, US2008/171855A1, US2008/241884A1, US2008/254512A1, US2008/260738A1, US2009/130106A1, US2009/148905A1, US2009/155275A1, US2009/162359A1, US2009/162360A1, US2009/175851A1, US2009/175867A1, US2009/232811A1, US2009/234105A1, US2009/263392A1, US2009/274649A1, EP346087A2, WO00/06605A2, WO02/072635A2, WO04/081051A1, WO06/020258A2, WO2007/044887A2, WO2007/095338A2, WO2007/137760A2, WO2008/119353A1, WO2009/021754A2, WO2009/068630A1, WO91/03493A1, WO93/23537A1, WO94/09131A1, WO94/12625A2, WO95/09917A1, WO96/37621A2, WO99/64460A1. The contents of the above-referenced applications are incorporated herein by reference in their entireties.

In one aspect, an isolated nucleic acid molecule encoding the antibody molecule, vectors and host cells thereof for producing the antibodies described herein are provided. The nucleic acid molecule includes but is not limited to RNA, genomic DNA and cDNA.

### Immuno-Oncology Therapeutic Agents

### Selected PD-1 Inhibitors

In various preferred embodiments I-O agents can be used as additional therapeutic agent with the PSMA therapeutic agent, such as radiolabeled Compound I, described herein. Any of the I-O agents described below in this section titled “Immuno-Oncology Therapeutic Agents” can be used with a PSMA therapeutic agent, such as radiolabeled Compound I described herein, to treat cancer preferably prostate cancer and other cancers that express prostate-specific membrane antigen (PSMA), e.g. PSMA expressing cancers such as PSMA over-expressing cancers and cancers that express PSMA in their neovasculature.

For example, PD-1 inhibitors can be used. The Programmed Death 1 (PD-1) protein is an inhibitory member of the extended CD28/CTLA-4 family of T cell regulators (Okazaki et al. (2002) Curr Opin Immunol 14: 391779-82; Bennett et al. (2003) J. Immunol. 170:711-8). Two ligands for PD-1 have been identified, PD-L1 (B7-H1) and PD-L2 (B7-DC), that have been shown to downregulate T cell activation upon binding to PD-1 (Freeman et al. (2000) J. Exp. Med. 192:1027-34; Carter et al. (2002) Eur. J. Immunol. 32:634-43). PD-L1 is abundant in a variety of human cancers (Dong et al. (2002) Nat. Med. 8:787-9).

PD-1 is known as an immunoinhibitory protein that negatively regulates TCR signals (Ishida, Y. et al. (1992) EMBO J. 11:3887-3895; Blank, C. et al. (Epub 2006 Dec. 29) Immunol. Immunother. 56(5):739-745). The interaction between PD-1 and PD-L1 can act as an immune checkpoint, which can lead to, e.g., a decrease in tumor infiltrating lymphocytes, a decrease in T-cell receptor mediated proliferation, and/or immune evasion by cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1 or PD-L2; the effect is additive when the interaction of PD-1 with PD-L2 is blocked as well (Iwai et al. (2002) Proc. Nat'l. Acad. Sci. USA 99:12293-7; Brown et al. (2003) J. Immunol. 170:1257-66).

In certain embodiments, a combination or method as described herein comprises a PD-1 inhibitor as I-O agent. In some embodiments, the PD-1 inhibitor is chosen from PDR001 (Novartis), Pembrolizumab (Merck & Co), Pidilizumab (CureTech), Durvalomab, Atezolizumab, Avelumab, Nivolumab (Bristol-Myers Squibb Company), MK-3475, MPDL3280A, MEDI4736, ipilimumab (Bristol-Myers Squibb Company), tremelimumab, MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is PDR001. PDR001 is also known as Spartalizumab. In other embodiments, the PD-1 inhibitor is not Pembrolizumab.

### Exemplary PD-1 Inhibitors

In one embodiment, the PD-1 inhibitor is an anti-PD-1 antibody molecule. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety. In some embodiments, the anti-PD-1 antibody molecule is Spartalizumab (PDR001).

In one embodiment, the anti-PD-1 antibody molecule comprises at least one, two, three, four, five or six complementarity determining regions (CDRs) (or collectively all of the CDRs) from a heavy and light chain variable region comprising an amino acid sequence shown in Table 1 (e.g., from the heavy and light chain variable region sequences of BAP049-Clone-E or BAP049-Clone-B disclosed in Table 1), or encoded by a nucleotide sequence shown in Table 1. In some embodiments, the CDRs are according to the Kabat definition (e.g., as set out in Table 1). In some embodiments, the CDRs are according to the Chothia definition (e.g., as set out in Table 1). In some embodiments, the CDRs are according to the combined CDR definitions of both Kabat and Chothia (e.g., as set out in Table 1). In one embodiment, the combination of Kabat and Chothia CDR of VH CDR1 comprises the amino acid sequence GYTFTTYWMH (SEQ ID NO: 39). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 1, or encoded by a nucleotide sequence shown in Table 1.

In one embodiment, the anti-PD-1 antibody molecule comprises a heavy chain variable region (VH) comprising a VHCDR1 amino acid sequence of SEQ ID NO: 1, a VHCDR2 amino acid sequence of SEQ ID NO: 2, and a VHCDR3 amino acid sequence of SEQ ID NO: 3; and a light chain variable region (VL) comprising a VLCDR1 amino acid sequence of SEQ ID NO: 10, a VLCDR2 amino acid sequence of SEQ ID NO: 11, and a VLCDR3 amino acid sequence of SEQ ID NO: 12, each disclosed in Table 1.

In one embodiment, the antibody molecule comprises a VH comprising a VHCDR1 encoded by the nucleotide sequence of SEQ ID NO: 24, a VHCDR2 encoded by the nucleotide sequence of SEQ ID NO: 25, and a VHCDR3 encoded by the nucleotide sequence of SEQ ID NO: 26; and a VL comprising a VLCDR1 encoded by the nucleotide sequence of SEQ ID NO: 29, a VLCDR2 encoded by the nucleotide sequence of SEQ ID NO: 30, and a VLCDR3 encoded by the nucleotide sequence of SEQ ID NO: 31, each disclosed in Table 1.

In one embodiment, the anti-PD-1 antibody molecule comprises a VH comprising the amino acid sequence of SEQ ID NO: 6, or an amino acid sequence at least 85%, 90%, 95%, 96%, 97%, 98%, or 99% identical or higher to SEQ ID NO: 6. In one embodiment, the anti-PD-1 antibody molecule comprises a VL comprising the amino acid sequence of SEQ ID NO: 20, or an amino acid sequence at least 85%, 90%, 95%, 96%, 97%, 98%, or 99% identical or higher to SEQ ID NO: 20. In one embodiment, the anti-PD-1 antibody molecule comprises a VL comprising the amino acid sequence of SEQ ID NO: 16, or an amino acid sequence at least 85%, 90%, 95%, 96%, 97%, 98%, or 99% identical or higher to SEQ ID NO: 16. In one embodiment, the anti-PD-1 antibody molecule comprises a VH comprising the amino acid sequence of SEQ ID NO: 6 and a VL comprising the amino acid sequence of SEQ ID NO: 20. In one embodiment, the anti-PD-1 antibody molecule comprises a VH comprising the amino acid sequence of SEQ ID NO: 6 and a VL comprising the amino acid sequence of SEQ ID NO: 16.

In one embodiment, the antibody molecule comprises a VH encoded by the nucleotide sequence of SEQ ID NO: 7, or a nucleotide sequence at least 85%, 90%, 95%, 96%, 97%, 98%, or 99% identical or higher to SEQ ID NO: 7. In one embodiment, the antibody molecule comprises a VL encoded by the nucleotide sequence of SEQ ID NO: 21 or 17, or a nucleotide sequence at least 85%, 90%, 95%, 96%, 97%, 98%, or 99% identical or higher to SEQ ID NO: 21 or 17. In one embodiment, the antibody molecule comprises a VH encoded by the nucleotide sequence of SEQ ID NO: 07 and a VL encoded by the nucleotide sequence of SEQ ID NO: 21 or 17.

In one embodiment, the anti-PD-1 antibody molecule comprises a heavy chain comprising the amino acid sequence of SEQ ID NO: 08, or an amino acid sequence at least 85%, 90%, 95%, 96%, 97%, 98%, or 99% identical or higher to SEQ ID NO: 08. In one embodiment, the anti-PD-1 antibody molecule comprises a light chain comprising the amino acid sequence of SEQ ID NO: 22, or an amino acid sequence at least 85%, 90%, 95%, 96%, 97%, 98%, or 99% identical or higher to SEQ ID NO: 22. In one embodiment, the anti-PD-1 antibody molecule comprises a light chain comprising the amino acid sequence of SEQ ID NO: 18, or an amino acid sequence at least 85%, 90%, 95%, 96%, 97%, 98%, or 99% identical or higher to SEQ ID NO: 18. In one embodiment, the anti-PD-1 antibody molecule comprises a heavy chain comprising the amino acid sequence of SEQ ID NO: 08 and a light chain comprising the amino acid sequence of SEQ ID NO: 22. In one embodiment, the anti-PD-1 antibody molecule comprises a heavy chain comprising the amino acid sequence of SEQ ID NO: 08 and a light chain comprising the amino acid sequence of SEQ ID NO: 18.

In one embodiment, the antibody molecule comprises a heavy chain encoded by the nucleotide sequence of SEQ ID NO: 9, or a nucleotide sequence at least 85%, 90%, 95%, 96%, 97%, 98%, or 99% identical or higher to SEQ ID NO: 9. In one embodiment, the antibody molecule comprises a light chain encoded by the nucleotide sequence of SEQ ID NO: 23 or 19, or a nucleotide sequence at least 85%, 90%, 95%, 96%, 97%, 98%, or 99% identical or higher to SEQ ID NO: 23 or 19. In one embodiment, the antibody molecule comprises a heavy chain encoded by the nucleotide sequence of SEQ ID NO: 9 and a light chain encoded by the nucleotide sequence of SEQ ID NO: 23 or 19.

The antibody molecules described herein can be made by vectors, host cells, and methods described in US 2015/0210769, incorporated by reference herein in its entirety.

TABLE 1
Amino acid and nucleotide sequences of exemplary
anti-PD-1 antibody molecules

| | | |
|---|---|---|
| BAP049-Clone-B HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 6 | VH | EVQLVQSGAEVKKPGESLRISCKGSGYTFTTYWMHW |
| | | VRQATGQGLEWMGNIYPGTGGSNFDEKFKNRVTITA |
| | | DKSTSTAYMELSSLRSEDTAVYYCTRWTTGTGAYWG |
| | | TQGTVTVSS |
| SEQ ID NO: 7 | DNA VH | GAGGTGCAGCTGGTGCAGTCAGGCGCCGAAGTGAAG |
| | | AAGCCCGGCGAGTCACTGAGAATTAGCTGTAAAGGT |
| | | TCAGGCTACACCTTCACTACCTACTGGATGCACTGG |
| | | GTCCGCCAGGCTACCGGTCAAGGCCTCGAGTGGATG |
| | | GGTAATATCTACCCCGGCACCGGCGGCTCTAACTTC |
| | | GACGAGAAGTTTAAGAATAGAGTGACTATCACCGCC |
| | | GATAAGTCTACTAGCACCGCCTATATGGAACTGTCT |
| | | AGCCTGAGATCAGAGGACACCGCCGTCTACTACTGC |
| | | ACTAGGTGGACTACCGGCACAGGCGCCTACTGGGGT |
| | | CAAGGCACTACCGTGACCGTGTCTAGC |
| SEQ ID NO: 8 | Heavy | EVQLVQSGAEVKKPGESLRISCKGSGYTFTTYWMHW |
| | chain | VRQATGQGLEWMGNIYPGTGGSNFDEKFKNRVTITA |
| | | DKSTSTAYMELSSLRSEDTAVYYCTRWTTGTGAYWG |
| | | QGTTVTVSSASTKGPSVFPLAPCSRSTSESTAALGC |
| | | LVKDYFPEPVTVSWNSGALTSGVHTFPAVLQSSGLY |
| | | SLSSVVTVPSSSLGTKTYTCNVDHKPSNTKVDKRVE |
| | | SKYGPPCPPCPAPEFLGGPSVFLFPPKPKDTLMISR |
| | | TPEVTCVVVDVSQEDPEVQFNWYVDGVEVHNAKTKP |
| | | REEQFNSTYRVVSVLTVLHQDWLNGKEYKCKVSNKG |
| | | LPSSIEKTISKAKGQPREPQVYTLPPSQEEMTKNQV |
| | | SLTCLVKGFYPSDIAVEWESNGQPENNYKTTPPVLD |
| | | SDGSFFLYSRLTVDKSRWQEGNVFSCSVMHEALHNH |
| | | YTQKSLSLSLG |
| SEQ ID NO: 9 | DNA | GAGGTGCAGCTGGTGCAGTCAGGCGCCGAAGTGAAG |
| | heavy | AAGCCCGGCGAGTCACTGAGAATTAGCTGTAAAGGT |
| | chain | TCAGGCTACACCTTCACTACCTACTGGATGCACTGG |
| | | GTCCGCCAGGCTACCGGTCAAGGCCTCGAGTGGATG |
| | | GGTAATATCTACCCCGGCACCGGCGGCTCTAACTTC |
| | | GACGAGAAGTTTAAGAATAGAGTGACTATCACCGCC |
| | | GATAAGTCTACTAGCACCGCCTATATGGAACTGTCT |
| | | AGCCTGAGATCAGAGGACACCGCCGTCTACTACTGC |
| | | ACTAGGTGGACTACCGGCACAGGCGCCTACTGGGGT |
| | | CAAGGCACTACCGTGACCGTGTCTAGCGCTAGCACT |
| | | AAGGGCCCGTCCGTGTTCCCCCTGGCACCTTGTAGC |
| | | CGGAGCACTAGCGAATCCACCGCTGCCCTCGGCTGC |
| | | CTGGTCAAGGATTACTTCCCGGAGCCCGTGACCGTG |
| | | TCCTGGAACAGCGGAGCCCTGACCTCCGGAGTGCAC |
| | | ACCTTCCCCGCTGTGCTGCAGAGCTCCGGGCTGTAC |
| | | TCGCTGTCGTCGGTGGTCACGGTGCCTTCATCTAGC |
| | | CTGGGTACCAAGACCTACACTTGCAACGTGGACCAC |
| | | AAGCCTTCCAACACTAAGGTGGACAAGCGCGTCGAA |
| | | TCGAAGTACGGCCCACCGTGCCCGCCTTGTCCCGCG |
| | | CCGGAGTTCCTCGGCGGTCCCTCGGTCTTTCTGTTC |
| | | CCACCGAAGCCCAAGGACACTTTGATGATTTCCCGC |
| | | ACCCCTGAAGTGACATGCGTGGTCGTGGACGTGTCA |
| | | CAGGAAGATCCGGAGGTGCAGTTCAATTGGTACGTG |
| | | GATGGCGTCGAGGTGCACAACGCCAAAACCAAGCCG |
| | | AGGGAGGAGCAGTTCAACTCCACTTACCGCGTCGTG |
| | | TCCGTGCTGACGGTGCTGCATCAGGACTGGCTGAAC |
| | | GGGAAGGAGTACAAGTGCAAAGTGTCCAACAAGGGA |
| | | CTTCCTAGCTCAATCGAAAAGACCATCTCGAAAGCC |
| | | AAGGGACAGCCCCGGGAACCCCAAGTGTATACCCTG |
| | | CCACCGAGCCAGGAAGAAATGACTAAGAACCAAGTC |
| | | TCATTGACTTGCCTTGTGAAGGGCTTCTACCCATCG |
| | | GATATCGCCGTGGAATGGGAGTCCAACGGCCAGCCG |
| | | GAAAACAACTACAAGACCACCCCTCCGGTGCTGGAC |
| | | TCAGACGGATCCTTCTTCCTCTACTCGCGGCTGACC |
| | | GTGGATAAGAGCAGATGGCAGGAGGGAAATGTGTTC |
| | | AGCTGTTCTGTGATGCATGAAGCCCTGCACAACCAC |
| | | TACACTCAGAAGTCCCTGTCCCTCTCCCTGGGA |
| BAP049-Clone-B LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 12 (Kabat) | LCDR3 | QNDYSYPYT |
| SEQ ID NO: 13 | LCDR1 | SQSLLDSGNQKNF |
| (Chothia) | | |
| SEQ ID NO: 14 | LCDR2 | WAS |
| (Chothia) | | |
| SEQ ID NO: 15 | LCDR3 | DYSYPY |
| (Chothia) | | |
| SEQ ID NO: 16 | VL | EIVLTQSPATLSLSPGERATLSCKSSQSLLDSGNQK |
| | | NFLTWYQQKPGKAPKLLIYWASTRESGVPSRFSGSG |
| | | SGTDFTFTISSLQPEDIATYYCQNDYSYPYTFGQGT |
| | | KVEIK |
| SEQ ID NO: 17 | DNA VL | GAGATCGTCCTGACTCAGTCACCCGCTACCCTGAGC |
| | | CTGAGCCCTGGCGAGCGGGCTACACTGAGCTGTAAA |
| | | TCTAGTCAGTCACTGCTGGATAGCGGTAATCAGAAG |
| | | AACTTCCTGACCTGGTATCAGCAGAAGCCCGGTAAA |
| | | GCCCCTAAGCTGCTGATCTACTGGGCCTCTACTAGA |
| | | GAATCAGGCGTGCCCTCTAGGTTTAGCGGTAGCGGT |
| | | AGTGGCACCGACTTCACCTTCACTATCTCTAGCCTG |
| | | CAGCCCGAGGATATCGCTACCTACTACTGTCAGAAC |
| | | GACTATAGCTACCCCTACACCTTCGGTCAAGGCACT |
| | | AAGGTCGAGATTAAG |
| SEQ ID NO: 18 | Light | EIVLTQSPATLSLSPGERATLSCKSSQSLLDSGNQK |
| | chain | NFLTWYQQKPGKAPKLLIYWASTRESGVPSRFSGSG |
| | | SGTDFTFTISSLQPEDIATYYCQNDYSYPYTFGQGT |
| | | KVEIKRTVAAPSVFIFPPSDEQLKSGTASVVCLLNN |
| | | FYPREAKVQWKVDNALQSGNSQESVTEQDSKDSTYS |
| | | LSSTLTLSKADYEKHKVYACEVTHQGLSSPVTKSFN |
| | | RGEC |
| SEQ ID NO: 19 | DNA light | GAGATCGTCCTGACTCAGTCACCCGCTACCCTGAGC |
| | chain | CTGAGCCCTGGCGAGCGGGCTACACTGAGCTGTAAA |
| | | TCTAGTCAGTCACTGCTGGATAGCGGTAATCAGAAG |
| | | AACTTCCTGACCTGGTATCAGCAGAAGCCCGGTAAA |
| | | GCCCCTAAGCTGCTGATCTACTGGGCCTCTACTAGA |
| | | GAATCAGGCGTGCCCTCTAGGTTTAGCGGTAGCGGT |
| | | AGTGGCACCGACTTCACCTTCACTATCTCTAGCCTG |
| | | CAGCCCGAGGATATCGCTACCTACTACTGTCAGAAC |
| | | GACTATAGCTACCCCTACACCTTCGGTCAAGGCACT |
| | | AAGGTCGAGATTAAGCGTACGGTGGCCGCTCCCAGC |
| | | GTGTTCATCTTCCCCCCCAGCGACGAGCAGCTGAAG |
| | | AGCGGCACCGCCAGCGTGGTGTGCCTGCTGAACAAC |
| | | TTCTACCCCCGGGAGGCCAAGGTGCAGTGGAAGGTG |
| | | GACAACGCCCTGCAGAGCGGCAACAGCCAGGAGAGC |
| | | GTCACCGAGCAGGACAGCAAGGACTCCACCTACAGC |
| | | CTGAGCAGCACCCTGACCCTGAGCAAGGCCGACTAC |
| | | GAGAAGCATAAGGTGTACGCCTGCGAGGTGACCCAC |
| | | CAGGGCCTGTCCAGCCCCGTGACCAAGAGCTTCAAC |
| | | AGGGGCGAGTGC |
| BAP049-Clone-E HC | | |
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 6 | VH | EVQLVQSGAEVKKPGESLRISCKGSGYTFTTYWMHW |
| | | VRQATGQGLEWMGNIYPGTGGSNFDEKFKNRVTITA |
| | | DKSTSTAYMELSSLRSEDTAVYYCTRWTTGTGAYWG |
| | | QGTTVTVSS |
| SEQ ID NO: 7 | DNA VH | GAGGTGCAGCTGGTGCAGTCAGGCGCCGAAGTGAAG |
| | | AAGCCCGGCGAGTCACTGAGAATTAGCTGTAAAGGT |
| | | TCAGGCTACACCTTCACTACCTACTGGATGCACTGG |
| | | GTCCGCCAGGCTACCGGTCAAGGCCTCGAGTGGATG |
| | | GGTAATATCTACCCCGGCACCGGCGGCTCTAACTTC |
| | | GACGAGAAGTTTAAGAATAGAGTGACTATCACCGCC |
| | | GATAAGTCTACTAGCACCGCCTATATGGAACTGTCT |
| | | AGCCTGAGATCAGAGGACACCGCCGTCTACTACTGC |
| | | ACTAGGTGGACTACCGGCACAGGCGCCTACTGGGGT |
| | | CAAGGCACTACCGTGACCGTGTCTAGC |
| SEQ ID NO: 8 | Heavy | EVQLVQSGAEVKKPGESLRISCKGSGYTFTTYWMHW |
| | chain | VRQATGQGLEWMGNIYPGTGGSNFDEKFKNRVTITA |
| | | DKSTSTAYMELSSLRSEDTAVYYCTRWTTGTGAYWG |
| | | QGTTVTVSSASTKGPSVFPLAPCSRSTSESTAALGC |
| | | LVKDYFPEPVTVSWNSGALTSGVHTFPAVLQSSGLY |
| | | SLSSVVTVPSSSLGTKTYTCNVDHKPSNTKVDKRVE |
| | | SKYGPPCPPCPAPEFLGGPSVFLFPPKPKDTLMISR |
| | | TPEVTCVVVDVSQEDPEVQFNWYVDGVEVHNAKTKP |
| | | REEQFNSTYRVVSVLTVLHQDWLNGKEYKCKVSNKG |
| | | LPSSIEKTISKAKGQPREPQVYTLPPSQEEMTKNQV |
| | | SLTCLVKGFYPSDIAVEWESNGQPENNYKTTPPVLD |
| | | SDGSFFLYSRLTVDKSRWQEGNVFSCSVMHEALHNH |
| | | YTQKSLSLSLG |
| SEQ ID NO: 9 | DNA | GAGGTGCAGCTGGTGCAGTCAGGCGCCGAAGTGAAG |
| | heavy | AAGCCCGGCGAGTCACTGAGAATTAGCTGTAAAGGT |
| | chain | TCAGGCTACACCTTCACTACCTACTGGATGCACTGG |
| | | GTCCGCCAGGCTACCGGTCAAGGCCTCGAGTGGATG |
| | | GGTAATATCTACCCCGGCACCGGCGGCTCTAACTTC |
| | | GACGAGAAGTTTAAGAATAGAGTGACTATCACCGCC |
| | | GATAAGTCTACTAGCACCGCCTATATGGAACTGTCT |
| | | AGCCTGAGATCAGAGGACACCGCCGTCTACTACTGC |
| | | ACTAGGTGGACTACCGGCACAGGCGCCTACTGGGGT |
| | | CAAGGCACTACCGTGACCGTGTCTAGCGCTAGCACT |
| | | AAGGGCCCGTCCGTGTTCCCCCTGGCACCTTGTAGC |
| | | CGGAGCACTAGCGAATCCACCGCTGCCCTCGGCTGC |
| | | CTGGTCAAGGATTACTTCCCGGAGCCCGTGACCGTG |
| | | TCCTGGAACAGCGGAGCCCTGACCTCCGGAGTGCAC |
| | | ACCTTCCCCGCTGTGCTGCAGAGCTCCGGGCTGTAC |
| | | TCGCTGTCGTCGGTGGTCACGGTGCCTTCATCTAGC |
| | | CTGGGTACCAAGACCTACACTTGCAACGTGGACCAC |
| | | AAGCCTTCCAACACTAAGGTGGACAAGCGCGTCGAA |
| | | TCGAAGTACGGCCCACCGTGCCCGCCTTGTCCCGCG |
| | | CCGGAGTTCCTCGGCGGTCCCTCGGTCTTTCTGTTC |
| | | CCACCGAAGCCCAAGGACACTTTGATGATTTCCCGC |
| | | ACCCCTGAAGTGACATGCGTGGTCGTGGACGTGTCA |
| | | CAGGAAGATCCGGAGGTGCAGTTCAATTGGTACGTG |
| | | GATGGCGTCGAGGTGCACAACGCCAAAACCAAGCCG |
| | | AGGGAGGAGCAGTTCAACTCCACTTACCGCGTCGTG |
| | | TCCGTGCTGACGGTGCTGCATCAGGACTGGCTGAAC |
| | | GGGAAGGAGTACAAGTGCAAAGTGTCCAACAAGGGA |
| | | CTTCCTAGCTCAATCGAAAAGACCATCTCGAAAGCC |
| | | AAGGGACAGCCCCGGGAACCCCAAGTGTATACCCTG |
| | | CCACCGAGCCAGGAAGAAATGACTAAGAACCAAGTC |
| | | TCATTGACTTGCCTTGTGAAGGGCTTCTACCCATCG |
| | | GATATCGCCGTGGAATGGGAGTCCAACGGCCAGCCG |
| | | GAAAACAACTACAAGACCACCCCTCCGGTGCTGGAC |
| | | TCAGACGGATCCTTCTTCCTCTACTCGCGGCTGACC |
| | | GTGGATAAGAGCAGATGGCAGGAGGGAAATGTGTTC |
| | | AGCTGTTCTGTGATGCATGAAGCCCTGCACAACCAC |
| | | TACACTCAGAAGTCCCTGTCCCTCTCCCTGGGA |
| BAP049-Clone-E LC | | |
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 12 (Kabat) | LCDR3 | QNDYSYPYT |
| SEQ ID NO: 13 | LCDR1 | SQSLLDSGNQKNF |
| (Chothia) | | |
| SEQ ID NO: 14 | LCDR2 | WAS |
| (Chothia) | | |
| SEQ ID NO: 15 | LCDR3 | DYSYPY |
| (Chothia) | | |
| SEQ ID NO: 20 | VL | EIVLTQSPATLSLSPGERATLSCKSSQSLLDSGNQK |
| | | NFLTWYQQKPGQAPRLLIYWASTRESGVPSRFSGSG |
| | | SGTDFTFTISSLEAEDAATYYCQNDYSYPYTFGQGT |
| | | KVEIK |
| SEQ ID NO: 21 | DNA VL | GAGATCGTCCTGACTCAGTCACCCGCTACCCTGAGC |
| | | CTGAGCCCTGGCGAGCGGGCTACACTGAGCTGTAAA |
| | | TCTAGTCAGTCACTGCTGGATAGCGGTAATCAGAAG |
| | | AACTTCCTGACCTGGTATCAGCAGAAGCCCGGTCAA |
| | | GCCCCTAGACTGCTGATCTACTGGGCCTCTACTAGA |
| | | GAATCAGGCGTGCCCTCTAGGTTTAGCGGTAGCGGT |
| | | AGTGGCACCGACTTCACCTTCACTATCTCTAGCCTG |
| | | GAAGCCGAGGACGCCGCTACCTACTACTGTCAGAAC |
| | | GACTATAGCTACCCCTACACCTTCGGTCAAGGCACT |
| | | AAGGTCGAGATTAAG |
| SEQ ID NO: 22 | Light | EIVLTQSPATLSLSPGERATLSCKSSQSLLDSGNQK |
| | chain | NFLTWYQQKPGQAPRLLIYWASTRESGVPSRFSGSG |
| | | SGTDFTFTISSLEAEDAATYYCONDYSYPYTFGQGT |
| | | KVEIKRTVAAPSVFIFPPSDEQLKSGTASVVCLLNN |
| | | FYPREAKVQWKVDNALQSGNSQESVTEQDSKDSTYS |
| | | LSSTLTLSKADYEKHKVYACEVTHQGLSSPVTKSFN |
| | | RGEC |
| SEQ ID NO: 23 | DNA light | GAGATCGTCCTGACTCAGTCACCCGCTACCCTGAGC |
| | chain | CTGAGCCCTGGCGAGCGGGCTACACTGAGCTGTAAA |
| | | TCTAGTCAGTCACTGCTGGATAGCGGTAATCAGAAG |
| | | AACTTCCTGACCTGGTATCAGCAGAAGCCCGGTCAA |
| | | GCCCCTAGACTGCTGATCTACTGGGCCTCTACTAGA |
| | | GAATCAGGCGTGCCCTCTAGGTTTAGCGGTAGCGGT |
| | | AGTGGCACCGACTTCACCTTCACTATCTCTAGCCTG |
| | | GAAGCCGAGGACGCCGCTACCTACTACTGTCAGAAC |
| | | GACTATAGCTACCCCTACACCTTCGGTCAAGGCACT |
| | | AAGGTCGAGATTAAGCGTACGGTGGCCGCTCCCAGC |
| | | GTGTTCATCTTCCCCCCCAGCGACGAGCAGCTGAAG |
| | | AGCGGCACCGCCAGCGTGGTGTGCCTGCTGAACAAC |
| | | TTCTACCCCCGGGAGGCCAAGGTGCAGTGGAAGGTG |
| | | GACAACGCCCTGCAGAGCGGCAACAGCCAGGAGAGC |
| | | GTCACCGAGCAGGACAGCAAGGACTCCACCTACAGC |
| | | CTGAGCAGCACCCTGACCCTGAGCAAGGCCGACTAC |
| | | GAGAAGCATAAGGTGTACGCCTGCGAGGTGACCCAC |
| | | CAGGGCCTGTCCAGCCCCGTGACCAAGAGCTTCAAC |
| | | AGGGGCGAGTGC |
| BAP049-Clone-B HC | | |
| SEQ ID NO: 24 (Kabat) | HCDR1 | ACCTACTGGATGCAC |
| SEQ ID NO: 25 (Kabat) | HCDR2 | AATATCTACCCCGGCACCGGCGGCTCTAACTTCGAC |
| | | GAGAAGTTTAAGAAT |
| SEQ ID NO: 26 (Kabat) | HCDR3 | TGGACTACCGGCACAGGCGCCTAC |
| SEQ NO: 27 | HCDR1 | GGCTACACCTTCACTACCTAC |
| (Chothia) | | |
| SEQ ID NO: 28 | HCDR2 | TACCCCGGCACCGGCGGC |
| (Chothia) | | |
| SEQ ID NO: 26 | HCDR3 | TGGACTACCGGCACAGGCGCCTAC |
| (Chothia) | | |
| BAP049-Clone-B LC | | |
| SEQ ID NO: 29 (Kabat) | LCDR1 | AAATCTAGTCAGTCACTGCTGGATAGCGGTAATCAG |
| | | AAGAACTTCCTGACC |
| SEQ ID NO: 30 (Kabat) | LCDR2 | TGGGCCTCTACTAGAGAATCA |
| SEQ ID NO: 31 (Kabat) | LCDR3 | CAGAACGACTATAGCTACCCCTACACC |
| SEQ ID NO: 32 | LCDR1 | AGTCAGTCACTGCTGGATAGCGGTAATCAGAAGAAC |
| (Chothia) | | TTC |
| SEQ ID NO: 33 | LCDR2 | TGGGCCTCT |
| (Chothia) | | |
| SEQ ID NO: 34 | LCDR3 | GACTATAGCTACCCCTAC |
| (Chothia) | | |
| BAP049-Clone-E HC | | |
| SEQ ID NO: 24 (Kabat) | HCDR1 | ACCTACTGGATGCAC |
| SEQ ID NO: 25 (Kabat) | HCDR2 | AATATCTACCCCGGCACCGGCGGCTCTAACTTCGAC |
| | | GAGAAGTTTAAGAAT |
| SEQ ID NO: 26 (Kabat) | HCDR3 | TGGACTACCGGCACAGGCGCCTAC |
| SEQ ID NO: 27 | HCDR1 | GGCTACACCTTCACTACCTAC |
| (Chothia) | | |
| SEQ ID NO: 28 | HCDR2 | TACCCCGGCACCGGCGGC |
| (Chothia) | | |
| SEQ ID NO: 26 | HCDR3 | TGGACTACCGGCACAGGCGCCTAC |
| (Chothia) | | |
| BAP049-Clone-E LC | | |
| SEQ ID NO: 29 (Kabat) | LCDR1 | AAATCTAGTCAGTCACTGCTGGATAGCGGTAATCAG |
| | | AAGAACTTCCTGACC |
| SEQ ID NO: 30 (Kabat) | LCDR2 | TGGGCCTCTACTAGAGAATCA |
| SEQ ID NO: 31 (Kabat) | LCDR3 | CAGAACGACTATAGCTACCCCTACACC |
| SEQ ID NO: 32 | LCDR1 | AGTCAGTCACTGCTGGATAGCGGTAATCAGAAGAAC |
| (Chothia) | | TTC |
| SEQ ID NO: 33 | LCDR2 | TGGGCCTCT |
| (Chothia) | | |
| SEQ ID NO: 34 | LCDR3 | GACTATAGCTACCCCTAC |
| (Chothia) | | |

In some embodiments, the PD-1 inhibitor is administered at a dose of about 200 mg to about 500 mg (e.g., about 300 mg to about 400 mg). In some embodiments, the PD-1 inhibitor is administered once every 3 weeks. In some embodiments, the PD-1 inhibitor is administered once every 4 weeks. In other embodiments, the PD-1 inhibitor is administered at a dose of about 200 mg to about 400 mg (e.g., about 300 mg) once every 3 weeks. In yet other embodiments, the PD-1 inhibitor is administered at a dose of about 300 mg to about 500 mg (e.g., about 400 mg) once every 4 weeks.

In some embodiments, the combination or method comprises a PD-1 inhibitor, e.g., PDR001, and a TGF-β inhibitor, e.g., NIS793. In some embodiments, this combination is administered to a subject in a therapeutically effective amount to treat, e.g., a prostate cancer.

In some embodiments, the combination or method comprises a PD-1 inhibitor, e.g., PDR001, and a TLR7 agonist, e.g., LHC165. In some embodiments, this combination is administered to a subject in a therapeutically effective amount to treat, e.g., a prostate cancer. In some embodiments, the TLR7 agonist, e.g., LHC165 is administered via intra-tumoral injection.

In some embodiments, the combination or method comprises a PD-1 inhibitor, e.g., PDR001, and an adenosine receptor antagonist, e.g., PBF509 (NIR178). In some embodiments, this combination is administered to a subject in a therapeutically effective amount to treat, e.g., a prostate cancer.

In some embodiments, the combination or method comprises a PD-1 inhibitor, e.g., PDR001, and an inhibitor of Porcupine, e.g., WNT974. In some embodiments, this combination is administered to a subject in a therapeutically effective amount to treat, e.g., a prostate cancer.

In some embodiments, the combination or method comprises a PD-1 inhibitor, e.g., PDR001, and an A2aR antagonist, e.g., PBF509 (NIR178). In some embodiments, this combination is administered to a subject in a therapeutically effective amount to treat, e.g., a prostate cancer. Without wishing to be bound by theory, it is believed that a combination or method comprising a PD-1 inhibitor, e.g., PDR001, and an A2aR antagonist, e.g., PBF509 (NIR178), can result in increased efficacy of the anti-PD-1 inhibitor. In some embodiments, the combination of a PD-1 inhibitor, e.g., PDR001, and an A2aR antagonist, e.g., PBF509 (NIR178), results in regression of a prostate tumor.

In some embodiments, the combination or method comprises a PD-1 inhibitor, e.g., PDR001, and a PD-L1 inhibitor, e.g., FAZ053. In some embodiments, the combination is administered to a subject in a therapeutically effective amount to treat, e.g., a prostate cancer.

### Other Exemplary PD-1 Inhibitors

In one embodiment, the anti-PD-1 antibody molecule is Pembrolizumab (Merck & Co), also known as Lambrolizumab, MK-3475, MK03475, SCH-900475, or KEYTRUDA®. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated herein by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab, e.g., as disclosed in Table 2. In another embodiment, the PD-1 inhibitor is not Pembrolizumab.

In one embodiment, the anti-PD-1 antibody molecule is Pidilizumab (CureTech), also known as CT-011. Pidilizumab and other anti-PD-1 antibodies are disclosed in Rosenblatt, J. et al. (2011) J Immunotherapy 34(5): 409-18, U.S. Pat. Nos. 7,695,715, 7,332,582, and 8,686,119, incorporated herein by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pidilizumab, e.g., as disclosed in Table 2.

In one embodiment, the anti-PD-1 antibody molecule is Durvalomab.

In one embodiment, the anti-PD-1 antibody molecule is Atezolizumab.

In one embodiment, the anti-PD-1 antibody molecule is Avelumab.

In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated herein by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MEDI0680.

In one embodiment, the anti-PD-1 antibody molecule is REGN2810 (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is BGB-A317 or BGB-108 (Beigene). In one embodiment, the anti-PD-1 antibody molecule comprises one or more of the CDR sequences (or collectively all of the CDR sequences), the heavy chain or light chain variable region sequence, or the heavy chain or light chain sequence of BGB-A317 or BGB-108.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANB011. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated herein by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated herein by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated herein by reference in their entirety).

In another embodiment, the PD-1 inhibitor is ipilimumab (Bristol-Myers Squibb Company). In yet another embodiment, the PD-1 inhibitor is nivolumab (Bristol-Myers Squibb Company). In one aspect, nivolumab is administered intravenously at doses of about 3 mg/kg every 2-3 weeks, for an initial period of two years. Thereafter, maintenance therapies every 12 weeks after the initial treatment can be used. It should be understood that these dosing regimens will vary according to the patient's response to the treatments and at the discretion of the treating clinician. In another embodiment, the dose of ipilimumab for the treatment of unresectable or metastatic melanoma is 3 mg/kg administered intravenously over 90 minutes every 3 weeks for a total of four doses. In other embodiments, the PD-1 inhibitor is selected from MK-3475, MPDL3280A, MEDI4736, and tremelimumab. In another embodiment, the PD-1 inhibitor is ipilimumab (Bristol-Myers Squibb Company) and the PSMA therapeutic agent is radiolabeled Compound I. In yet another embodiment, the PD-1 inhibitor is nivolumab (Bristol-Myers Squibb Company) and the PSMA therapeutic agent is radiolabeled Compound I. In still another embodiment, the PD-1 inhibitor is tremelimumab and the PSMA therapeutic agent is radiolabeled Compound I.

TABLE 2
Amino acid sequences of other exemplary
anti-PD-1 antibody molecules

| | | |
|---|---|---|
| Pembrolizumab | | |
| SEQ ID NO: 35 | Heavy chain | QVQLVQSGVEVKKPGASVKVSCKASGYTFTNYYMYWVR |
| | | QAPGQGLEWMGGINPSNGGTNFNEKFKNRVTLTTDSST |
| | | TTAYMELKSLQFDDTAVYYCARRDYRFDMGFDYWGQGT |
| | | TVTVSSASTKGPSVFPLAPCSRSTSESTAALGCLVKDY |
| | | FPEPVTVSWNSGALTSGVHTFPAVLQSSGLYSLSSVVT |
| | | VPSSSLGTKTYTCNVDHKPSNTKVDKRVESKYGPPCPP |
| | | CPAPEFLGGPSVFLFPPKPKDTLMISRTPEVTCVVVDV |
| | | SQEDPEVQFNWYVDGVEVHNAKTKPREEQFNSTYRVVS |
| | | VLTVLHQDWLNGKEYKCKVSNKGLPSSIEKTISKAKGQ |
| | | PREPQVYTLPPSQEEMTKNQVSLTCLVKGFYPSDIAVE |
| | | WESNGQPENNYKTTPPVLDSDGSFFLYSRLTVDKSRWQ |
| | | EGNVFSCSVMHEALHNHYTQKSLSLSLGK |
| SEQ ID NO: 36 | Light chain | EIVLTQSPATLSLSPGERATLSCRASKGVSTSGYSYLH |
| | | WYQQKPGQAPRLLIYLASYLESGVPARFSGSGSGTDFT |
| | | LTISSLEPEDFAVYYCQHSRDLPLTFGGGTKVEIKRTV |
| | | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |
| Pidilizumab | | |
| SEQ ID NO: 37 | Heavy chain | QVQLVQSGSELKKPGASVKISCKASGYTFTNYGMNWVR |
| | | QAPGQGLQWMGWINTDSGESTYAEEFKGRFVFSLDTSV |
| | | NTAYLQITSLTAEDTGMYFCVRVGYDALDYWGQGTLVT |
| | | VSSASTKGPSVFPLAPSSKSTSGGTAALGCLVKDYFPE |
| | | PVTVSWNSGALTSGVHTFPAVLQSSGLYSLSSVVTVPS |
| | | SSLGTQTYICNVNHKPSNTKVDKRVEPKSCDKTHTCPP |
| | | CPAPELLGGPSVFLFPPKPKDTLMISRTPEVTCVVVDV |
| | | SHEDPEVKFNWYVDGVEVHNAKTKPREEQYNSTYRVVS |
| | | VLTVLHQDWLNGKEYKCKVSNKALPAPIEKTISKAKGQ |
| | | PREPQVYTLPPSREEMTKNQVSLTCLVKGFYPSDIAVE |
| | | WESNGQPENNYKTTPPVLDSDGSFFLYSKLTVDKSRWQ |
| | | QGNVFSCSVMHEALHNHYTQKSLSLSPGK |
| SEQ ID NO: 38 | Light chain | EIVLTQSPSSLSASVGDRVTITCSARSSVSYMHWFQQK |
| | | PGKAPKLWIYRTSNLASGVPSRFSGSGSGTSYCLTINS |
| | | LQPEDFATYYCQQRSSFPLTFGGGTKLEIKRTVAAPSV |
| | | FIFPPSDEQLKSGTASVVCLLNNFYPREAKVQWKVDNA |
| | | LQSGNSQESVTEQDSKDSTYSLSSTLTLSKADYEKHKV |
| | | YACEVTHQGLSSPVTKSFNRGEC |

### Additional Combination Therapies

In an embodiment, the combination or method comprises a PD-1 inhibitor (e.g., PDR001), and an mTOR inhibitor, e.g., RAD001 (also known as everolimus). In some embodiments, the combination comprises PDR001 and an mTOR inhibitor, e.g., RAD001. In some embodiments, the combination comprises PDR001 and RAD001. In some embodiments, the mTOR inhibitor, e.g., RAD001, is administered once weekly at a dose of at least 0.5 mg, 1 mg, 2 mg, 3 mg, 4 mg, 5 mg, 6 mg, 7 mg, 8 mg, 9 mg, or 10 mgs. In some embodiments, the mTOR inhibitor, e.g., RAD001, is administered once weekly at a dose of 10 mg. In some embodiments, the mTOR inhibitor, e.g., RAD001, is administered once weekly at a dose of 5 mg. In some embodiments, the mTOR inhibitor, e.g., RAD001, is administered once daily at a dose of at least 0.5 mg, 1 mg, 2 mg, 3 mg, 4 mg, 5 mg, 6 mg, 7 mg, 8 mg, 9 mg, or 10 mgs. In some embodiments, the mTOR inhibitor, e.g., RAD001, is administered once daily at a dose of 0.5 mg. In some embodiments, this combination is administered to a subject in a therapeutically effective amount to treat a cancer, e.g., a cancer described herein, e.g., a prostate cancer.

### LAG-3 Inhibitors

In certain embodiments, the combination or method according to the invention comprising a PSMA therapeutic agent as described herein comprises a LAG-3 inhibitor as I-O therapeutic agent. In some embodiments, the LAG-3 inhibitor is chosen from LAG525 (Novartis), BMS-986016 (Bristol-Myers Squibb), or TSR-033 (Tesaro).

### Exemplary LAG-3 Inhibitors

In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated herein by reference in its entirety.

In one embodiment, the anti-LAG-3 antibody molecule comprises at least one, two, three, four, five or six complementarity determining regions (CDRs) (or collectively all of the CDRs) from a heavy and light chain variable region comprising an amino acid sequence shown in Table 3 (e.g., from the heavy and light chain variable region sequences of BAP050-Clone I or BAP050-Clone J disclosed in Table 3), or encoded by a nucleotide sequence shown in Table 5. In some embodiments, the CDRs are according to the Kabat definition (e.g., as set out in Table 3). In some embodiments, the CDRs are according to the Chothia definition (e.g., as set out in Table 3). In some embodiments, the CDRs are according to the combined CDR definitions of both Kabat and Chothia (e.g., as set out in Table 3). In one embodiment, the combination of Kabat and Chothia CDR of VH CDR1 comprises the amino acid sequence GFTLTNYGMN (SEQ ID NO: 105). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 3, or encoded by a nucleotide sequence shown in Table 3.

In one embodiment, the anti-LAG-3 antibody molecule comprises a heavy chain variable region (VH) comprising a VHCDR1 amino acid sequence of SEQ ID NO: 40, a VHCDR2 amino acid sequence of SEQ ID NO: 41, and a VHCDR3 amino acid sequence of SEQ ID NO: 42; and a light chain variable region (VL) comprising a VLCDR1 amino acid sequence of SEQ ID NO: 49, a VLCDR2 amino acid sequence of SEQ ID NO: 50, and a VLCDR3 amino acid sequence of SEQ ID NO: 51, each disclosed in Table 3.

In one embodiment, the anti-LAG-3 antibody molecule comprises a VH comprising a VHCDR1 encoded by the nucleotide sequence of SEQ ID NO: 75 or 76, a VHCDR2 encoded by the nucleotide sequence of SEQ ID NO: 77 or 78, and a VHCDR3 encoded by the nucleotide sequence of SEQ ID NO: 79 or 80; and a VL comprising a VLCDR1 encoded by the nucleotide sequence of SEQ ID NO: 85 or 86, a VLCDR2 encoded by the nucleotide sequence of SEQ ID NO: 87 or 88, and a VLCDR3 encoded by the nucleotide sequence of SEQ ID NO: 89 or 90, each disclosed in Table 3. In one embodiment, the anti-LAG-3 antibody molecule comprises a VH comprising a VHCDR1 encoded by the nucleotide sequence of SEQ ID NO: 97 or 76, a VHCDR2 encoded by the nucleotide sequence of SEQ ID NO: 98 or 78, and a VHCDR3 encoded by the nucleotide sequence of SEQ ID NO: 99 or 80; and a VL comprising a VLCDR1 encoded by the nucleotide sequence of SEQ ID NO: 85 or 86, a VLCDR2 encoded by the nucleotide sequence of SEQ ID NO: 87 or 88, and a VLCDR3 encoded by the nucleotide sequence of SEQ ID NO: 89 or 90, each disclosed in Table 3.

In one embodiment, the anti-LAG-3 antibody molecule comprises a VH comprising the amino acid sequence of SEQ ID NO: 45, or an amino acid sequence at least 85%, 90%, 95%, 96%, 97%, 98%, or 99% identical or higher to SEQ ID NO: 45. In one embodiment, the anti-LAG-3 antibody molecule comprises a VL comprising the amino acid sequence of SEQ ID NO: 57, or an amino acid sequence at least 85%, 90%, 95%, 96%, 97%, 98%, or 99% identical or higher to SEQ ID NO: 57. In one embodiment, the anti-LAG-3 antibody molecule comprises a VH comprising the amino acid sequence of SEQ ID NO: 63, or an amino acid sequence at least 85%, 90%, 95%, 96%, 97%, 98%, or 99% identical or higher to SEQ ID NO: 63. In one embodiment, the anti-LAG-3 antibody molecule comprises a VL comprising the amino acid sequence of SEQ ID NO: 69, or an amino acid sequence at least 85%, 90%, 95%, 96%, 97%, 98%, or 99% identical or higher to SEQ ID NO: 69. In one embodiment, the anti-LAG-3 antibody molecule comprises a VH comprising the amino acid sequence of SEQ ID NO: 45 and a VL comprising the amino acid sequence of SEQ ID NO: 57. In one embodiment, the anti-LAG-3 antibody molecule comprises a VH comprising the amino acid sequence of SEQ ID NO: 63 and a VL comprising the amino acid sequence of SEQ ID NO: 69.

In one embodiment, the antibody molecule comprises a VH encoded by the nucleotide sequence of SEQ ID NO: 46 or 47, or a nucleotide sequence at least 85%, 90%, 95%, 96%, 97%, 98%, or 99% identical or higher to SEQ ID NO: 46 or 47. In one embodiment, the antibody molecule comprises a VL encoded by the nucleotide sequence of SEQ ID NO: 58 or 59, or a nucleotide sequence at least 85%, 90%, 95%, 96%, 97%, 98%, or 99% identical or higher to SEQ ID NO: 58 or 59. In one embodiment, the antibody molecule comprises a VH encoded by the nucleotide sequence of SEQ ID NO: 64 or 65, or a nucleotide sequence at least 85%, 90%, 95%, 96%, 97%, 98%, or 99% identical or higher to SEQ ID NO: 64 or 65. In one embodiment, the antibody molecule comprises a VL encoded by the nucleotide sequence of SEQ ID NO: 70 or 71, or a nucleotide sequence at least 85%, 90%, 95%, 96%, 97%, 98%, or 99% identical or higher to SEQ ID NO: 70 or 71. In one embodiment, the antibody molecule comprises a VH encoded by the nucleotide sequence of SEQ ID NO: 46 or 47 and a VL encoded by the nucleotide sequence of SEQ ID NO: 58 or 59. In one embodiment, the antibody molecule comprises a VH encoded by the nucleotide sequence of SEQ ID NO: 64 or 65 and a VL encoded by the nucleotide sequence of SEQ ID NO: 70 or 71.

In one embodiment, the anti-LAG-3 antibody molecule comprises a heavy chain comprising the amino acid sequence of SEQ ID NO: 48, or an amino acid sequence at least 85%, 90%, 95%, 96%, 97%, 98%, or 99% identical or higher to SEQ ID NO: 48. In one embodiment, the anti-LAG-3 antibody molecule comprises a light chain comprising the amino acid sequence of SEQ ID NO: 60, or an amino acid sequence at least 85%, 90%, 95%, 96%, 97%, 98%, or 99% identical or higher to SEQ ID NO: 60. In one embodiment, the anti-LAG-3 antibody molecule comprises a heavy chain comprising the amino acid sequence of SEQ ID NO: 66, or an amino acid sequence at least 85%, 90%, 95%, 96%, 97%, 98%, or 99% identical or higher to SEQ ID NO: 66. In one embodiment, the anti-LAG-3 antibody molecule comprises a light chain comprising the amino acid sequence of SEQ ID NO: 72, or an amino acid sequence at least 85%, 90%, 95%, 96%, 97%, 98%, or 99% identical or higher to SEQ ID NO: 72. In one embodiment, the anti-LAG-3 antibody molecule comprises a heavy chain comprising the amino acid sequence of SEQ ID NO: 48 and a light chain comprising the amino acid sequence of SEQ ID NO: 60. In one embodiment, the anti-LAG-3 antibody molecule comprises a heavy chain comprising the amino acid sequence of SEQ ID NO: 66 and a light chain comprising the amino acid sequence of SEQ ID NO: 72.

In one embodiment, the antibody molecule comprises a heavy chain encoded by the nucleotide sequence of SEQ ID NO: 55 or 56, or a nucleotide sequence at least 85%, 90%, 95%, 96%, 97%, 98%, or 99% identical or higher to SEQ ID NO: 55 or 56. In one embodiment, the antibody molecule comprises a light chain encoded by the nucleotide sequence of SEQ ID NO: 61 or 62, or a nucleotide sequence at least 85%, 90%, 95%, 96%, 97%, 98%, or 99% identical or higher to SEQ ID NO: 61 or 62. In one embodiment, the antibody molecule comprises a heavy chain encoded by the nucleotide sequence of SEQ ID NO: 67 or 68, or a nucleotide sequence at least 85%, 90%, 95%, 96%, 97%, 98%, or 99% identical or higher to SEQ TD NO: 67 or 68. In one embodiment, the antibody molecule comprises a light chain encoded by the nucleotide sequence of SEQ ID NO: 73 or 74, or a nucleotide sequence at least 85%, 90%, 95%, 96%, 97%, 98%, or 99% identical or higher to SEQ ID NO: 73 or 74. In one embodiment, the antibody molecule comprises a heavy chain encoded by the nucleotide sequence of SEQ TD NO: 55 or 56 and a light chain encoded by the nucleotide sequence of SEQ ID NO: 61 or 62. In one embodiment, the antibody molecule comprises a heavy chain encoded by the nucleotide sequence of SEQ ID NO: 67 or 68 and a light chain encoded by the nucleotide sequence of SEQ ID NO: 73 or 74.

The antibody molecules described herein can be made by vectors, host cells, and methods described in US 2015/0259420, incorporated herein by reference in its entirety.

TABLE 3
Amino acid and nucleotide sequences of
exemplary anti-LAG-3 antibody molecules

| | | |
|---|---|---|
| BAP050-Clone I HC | | |
| SEQ ID NO: 40 | HCDR1 | NYGMN |
| (Kabat) | | |
| SEQ ID NO: 41 | HCDR2 | WINTDTGEPTYADDFKG |
| (Kabat) | | |
| SEQ ID NO: 42 | HCDR3 | NPPYYYGTNNAEAMDY |
| (Kabat) | | |
| SEQ ID NO: 43 | HCDR1 | GFTLTNY |
| (Chothia) | | |
| SEQ ID NO: 44 | HCDR2 | NTDTGE |
| (Chothia) | | |
| SEQ ID NO: 42 | HCDR3 | NPPYYYGTNNAEAMDY |
| (Chothia) | | |
| SEQ ID NO: 45 | VH | QVQLVQSGAEVKKPGASVKVSCKASGFTLTNYG |
| | | MNWVRQARGQRLEWIGWINTDTGEPTYADDFKG |
| | | RFVFSLDTSVSTAYLQISSLKAEDTAVYYCARN |
| | | PPYYYGTNNAEAMDYWGQGTTVTVSS |
| SEQ ID NO: 46 | DNA | CAAGTGCAGCTGGTGCAGTCGGGAGCCGAAGTG |
| | VH | AAGAAGCCTGGAGCCTCGGTGAAGGTGTCGTGC |
| | | AAGGCATCCGGATTCACCCTCACCAATTACGGG |
| | | ATGAACTGGGTCAGACAGGCCCGGGGTCAACGG |
| | | CTGGAGTGGATCGGATGGATTAACACCGACACC |
| | | GGGGAGCCTACCTACGCGGACGATTTCAAGGGA |
| | | CGGTTCGTGTTCTCCCTCGACACCTCCGTGTCC |
| | | ACCGCCTACCTCCAAATCTCCTCACTGAAAGCG |
| | | GAGGACACCGCCGTGTACTATTGCGCGAGGAAC |
| | | CCGCCCTACTACTACGGAACCAACAACGCCGAA |
| | | GCCATGGACTACTGGGGCCAGGGCACCACTGTG |
| | | ACTGTGTCCAGC |
| SEQ ID NO: 47 | DNA | CAGGTGCAGCTGGTGCAGTCTGGCGCCGAAGTG |
| | VH | AAGAAACCTGGCGCCTCCGTGAAGGTGTCCTGC |
| | | AAGGCCTCTGGCTTCACCCTGACCAACTACGGC |
| | | ATGAACTGGGTGCGACAGGCCAGGGGCCAGCGG |
| | | CTGGAATGGATCGGCTGGATCAACACCGACACC |
| | | GGCGAGCCTACCTACGCCGACGACTTCAAGGGC |
| | | AGATTCGTGTTCTCCCTGGACACCTCCGTGTCC |
| | | ACCGCCTACCTGCAGATCTCCAGCCTGAAGGCC |
| | | GAGGATACCGCCGTGTACTACTGCGCCCGGAAC |
| | | CCCCCTTACTACTACGGCACCAACAACGCCGAG |
| | | GCCATGGACTATTGGGGCCAGGGCACCACCGTG |
| | | ACCGTGTCCTCT |
| SEQ ID NO: 48 | Heavy | QVQLVQSGAEVKKPGASVKVSCKASGFTLTNYG |
| | chain | MNWVRQARGQRLEWIGWINTDTGEPTYADDFKG |
| | | RFVFSLDTSVSTAYLQISSLKAEDTAVYYCARN |
| | | PPYYYGTNNAEAMDYWGQGTTVTVSSASTKGPS |
| | | VFPLAPCSRSTSESTAALGCLVKDYFPEPVTVS |
| | | WNSGALTSGVHTFPAVLQSSGLYSLSSVVTVPS |
| | | SSLGTKTYTCNVDHKPSNTKVDKRVESKYGPPC |
| | | PPCPAPEFLGGPSVFLFPPKPKDTLMISRTPEV |
| | | TCVVVDVSQEDPEVQFNWYVDGVEVHNAKTKPR |
| | | EEQFNSTYRVVSVLTVLHQDWLNGKEYKCKVSN |
| | | KGLPSSIEKTISKAKGQPREPQVYTLPPSQEEM |
| | | TKNQVSLTCLVKGFYPSDIAVEWESNGQPENNY |
| | | KTTPPVLDSDGSFFLYSRLTVDKSRWQEGNVFS |
| | | CSVMHEALHNHYTQKSLSLSLG |
| SEQ ID NO: 55 | DNA | CAAGTGCAGCTGGTGCAGTCGGGAGCCGAAGTG |
| | heavy | AAGAAGCCTGGAGCCTCGGTGAAGGTGTCGTGC |
| | chain | AAGGCATCCGGATTCACCCTCACCAATTACGGG |
| | | ATGAACTGGGTCAGACAGGCCCGGGGTCAACGG |
| | | CTGGAGTGGATCGGATGGATTAACACCGACACC |
| | | GGGGAGCCTACCTACGCGGACGATTTCAAGGGA |
| | | CGGTTCGTGTTCTCCCTCGACACCTCCGTGTCC |
| | | ACCGCCTACCTCCAAATCTCCTCACTGAAAGCG |
| | | GAGGACACCGCCGTGTACTATTGCGCGAGGAAC |
| | | CCGCCCTACTACTACGGAACCAACAACGCCGAA |
| | | GCCATGGACTACTGGGGCCAGGGCACCACTGTG |
| | | ACTGTGTCCAGCGCGTCCACTAAGGGCCCGTCC |
| | | GTGTTCCCCCTGGCACCTTGTAGCCGGAGCACT |
| | | AGCGAATCCACCGCTGCCCTCGGCTGCCTGGTC |
| | | AAGGATTACTTCCCGGAGCCCGTGACCGTGTCC |
| | | TGGAACAGCGGAGCCCTGACCTCCGGAGTGCAC |
| | | ACCTTCCCCGCTGTGCTGCAGAGCTCCGGGCTG |
| | | TACTCGCTGTCGTCGGTGGTCACGGTGCCTTCA |
| | | TCTAGCCTGGGTACCAAGACCTACACTTGCAAC |
| | | GTGGACCACAAGCCTTCCAACACTAAGGTGGAC |
| | | AAGCGCGTCGAATCGAAGTACGGCCCACCGTGC |
| | | CCGCCTTGTCCCGCGCCGGAGTTCCTCGGCGGT |
| | | CCCTCGGTCTTTCTGTTCCCACCGAAGCCCAAG |
| | | GACACTTTGATGATTTCCCGCACCCCTGAAGTG |
| | | ACATGCGTGGTCGTGGACGTGTCACAGGAAGAT |
| | | CCGGAGGTGCAGTTCAATTGGTACGTGGATGGC |
| | | GTCGAGGTGCACAACGCCAAAACCAAGCCGAGG |
| | | GAGGAGCAGTTCAACTCCACTTACCGCGTCGTG |
| | | TCCGTGCTGACGGTGCTGCATCAGGACTGGCTG |
| | | AACGGGAAGGAGTACAAGTGCAAAGTGTCCAAC |
| | | AAGGGACTTCCTAGCTCAATCGAAAAGACCATC |
| | | TCGAAAGCCAAGGGACAGCCCCGGGAACCCCAA |
| | | GTGTATACCCTGCCACCGAGCCAGGAAGAAATG |
| | | ACTAAGAACCAAGTCTCATTGACTTGCCTTGTG |
| | | AAGGGCTTCTACCCATCGGATATCGCCGTGGAA |
| | | TGGGAGTCCAACGGCCAGCCGGAAAACAACTAC |
| | | AAGACCACCCCTCCGGTGCTGGACTCAGACGGA |
| | | TCCTTCTTCCTCTACTCGCGGCTGACCGTGGAT |
| | | AAGAGCAGATGGCAGGAGGGAAATGTGTTCAGC |
| | | TGTTCTGTGATGCATGAAGCCCTGCACAACCAC |
| | | TACACTCAGAAGTCCCTGTCCCTCTCCCTGGGA |
| SEQ ID NO: 56 | DNA | CAGGTGCAGCTGGTGCAGTCTGGCGCCGAAGTG |
| | heavy | AAGAAACCTGGCGCCTCCGTGAAGGTGTCCTGC |
| | chain | AAGGCCTCTGGCTTCACCCTGACCAACTACGGC |
| | | ATGAACTGGGTGCGACAGGCCAGGGGCCAGCGG |
| | | CTGGAATGGATCGGCTGGATCAACACCGACACC |
| | | GGCGAGCCTACCTACGCCGACGACTTCAAGGGC |
| | | AGATTCGTGTTCTCCCTGGACACCTCCGTGTCC |
| | | ACCGCCTACCTGCAGATCTCCAGCCTGAAGGCC |
| | | GAGGATACCGCCGTGTACTACTGCGCCCGGAAC |
| | | CCCCCTTACTACTACGGCACCAACAACGCCGAG |
| | | GCCATGGACTATTGGGGCCAGGGCACCACCGTG |
| | | ACCGTGTCCTCTGCTTCTACCAAGGGGCCCAGC |
| | | GTGTTCCCCCTGGCCCCCTGCTCCAGAAGCACC |
| | | AGCGAGAGCACAGCCGCCCTGGGCTGCCTGGTG |
| | | AAGGACTACTTCCCCGAGCCCGTGACCGTGTCC |
| | | TGGAACAGCGGAGCCCTGACCAGCGGCGTGCAC |
| | | ACCTTCCCCGCCGTGCTGCAGAGCAGCGGCCTG |
| | | TACAGCCTGAGCAGCGTGGTGACCGTGCCCAGC |
| | | AGCAGCCTGGGCACCAAGACCTACACCTGTAAC |
| | | GTGGACCACAAGCCCAGCAACACCAAGGTGGAC |
| | | AAGAGGGTGGAGAGCAAGTACGGCCCACCCTGC |
| | | CCCCCCTGCCCAGCCCCCGAGTTCCTGGGCGGA |
| | | CCCAGCGTGTTCCTGTTCCCCCCCAAGCCCAAG |
| | | GACACCCTGATGATCAGCAGAACCCCCGAGGTG |
| | | ACCTGTGTGGTGGTGGACGTGTCCCAGGAGGAC |
| | | CCCGAGGTCCAGTTCAACTGGTACGTGGACGGC |
| | | GTGGAGGTGCACAACGCCAAGACCAAGCCCAGA |
| | | GAGGAGCAGTTTAACAGCACCTACCGGGTGGTG |
| | | TCCGTGCTGACCGTGCTGCACCAGGACTGGCTG |
| | | AACGGCAAAGAGTACAAGTGTAAGGTCTCCAAC |
| | | AAGGGCCTGCCAAGCAGCATCGAAAAGACCATC |
| | | AGCAAGGCCAAGGGCCAGCCTAGAGAGCCCCAG |
| | | GTCTACACCCTGCCACCCAGCCAAGAGGAGATG |
| | | ACCAAGAACCAGGTGTCCCTGACCTGTCTGGTG |
| | | AAGGGCTTCTACCCAAGCGACATCGCCGTGGAG |
| | | TGGGAGAGCAACGGCCAGCCCGAGAACAACTAC |
| | | AAGACCACCCCCCCAGTGCTGGACAGCGACGGC |
| | | AGCTTCTTCCTGTACAGCAGGCTGACCGTGGAC |
| | | AAGTCCAGATGGCAGGAGGGCAACGTCTTTAGC |
| | | TGCTCCGTGATGCACGAGGCCCTGCACAACCAC |
| | | TACACCCAGAAGAGCCTGAGCCTGTCCCTGGGC |
| BAP050-Clone I LC | | |
| SEQ ID NO: 49 | LCDR1 | SSSQDISNYLN |
| (Kabat) | | |
| SEQ ID NO: 50 | LCDR2 | YTSTLHL |
| (Kabat) | | |
| SEQ ID NO: 51 | LCDR3 | QQYYNLPWT |
| (Kabat) | | |
| SEQ ID NO: 52 | LCDR1 | SQDISNY |
| (Chothia) | | |
| SEQ ID NO: 53 | LCDR2 | YTS |
| (Chothia) | | |
| SEQ ID NO: 54 | LCDR3 | YYNLPW |
| (Chothia) | | |
| SEQ ID NO: 57 | VL | DIQMTQSPSSLSASVGDRVTITCSSSQDISNYL |
| | | NWYLQKPGQSPQLLIYYTSTLHLGVPSRFSGSG |
| | | SGTEFTLTISSLQPDDFATYYCQQYYNLPWTFG |
| | | QGTKVEIK |
| SEQ ID NO: 58 | DNA | GATATTCAGATGACTCAGTCACCTAGTAGCCTG |
| | VL | AGCGCTAGTGTGGGCGATAGAGTGACTATCACC |
| | | TGTAGCTCTAGTCAGGATATCTCTAACTACCTG |
| | | AACTGGTATCTGCAGAAGCCCGGTCAATCACCT |
| | | CAGCTGCTGATCTACTACACTAGCACCCTGCAC |
| | | CTGGGCGTGCCCTCTAGGTTTAGCGGTAGCGGT |
| | | AGTGGCACCGAGTTCACCCTGACTATCTCTAGC |
| | | CTGCAGCCCGACGACTTCGCTACCTACTACTGT |
| | | CAGCAGTACTATAACCTGCCCTGGACCTTCGGT |
| | | CAAGGCACTAAGGTCGAGATTAAG |
| SEQ ID NO: 59 | DNA | GACATCCAGATGACCCAGTCCCCCTCCAGCCTG |
| | VL | TCTGCTTCCGTGGGCGACAGAGTGACCATCACC |
| | | TGTTCCTCCAGCCAGGACATCTCCAACTACCTG |
| | | AACTGGTATCTGCAGAAGCCCGGCCAGTCCCCT |
| | | CAGCTGCTGATCTACTACACCTCCACCCTGCAC |
| | | CTGGGCGTGCCCTCCAGATTTTCCGGCTCTGGC |
| | | TCTGGCACCGAGTTTACCCTGACCATCAGCTCC |
| | | CTGCAGCCCGACGACTTCGCCACCTACTACTGC |
| | | CAGCAGTACTACAACCTGCCCTGGACCTTCGGC |
| | | CAGGGCACCAAGGTGGAAATCAAG |
| SEQ ID NO: 60 | Light | DIQMTQSPSSLSASVGDRVTITCSSSQDISNYL |
| | chain | NWYLQKPGQSPQLLIYYTSTLHLGVPSRFSGSG |
| | | SGTEFTLTISSLQPDDFATYYCQQYYNLPWTFG |
| | | QGTKVEIKRTVAAPSVFIFPPSDEQLKSGTASV |
| | | VCLLNNFYPREAKVQWKVDNALQSGNSQESVTE |
| | | QDSKDSTYSLSSTLTLSKADYEKHKVYACEVTH |
| | | QGLSSPVTKSFNRGEC |
| SEQ ID NO: 61 | DNA | GATATTCAGATGACTCAGTCACCTAGTAGCCTG |
| | light | AGCGCTAGTGTGGGCGATAGAGTGACTATCACC |
| | chain | TGTAGCTCTAGTCAGGATATCTCTAACTACCTG |
| | | AACTGGTATCTGCAGAAGCCCGGTCAATCACCT |
| | | CAGCTGCTGATCTACTACACTAGCACCCTGCAC |
| | | CTGGGCGTGCCCTCTAGGTTTAGCGGTAGCGGT |
| | | AGTGGCACCGAGTTCACCCTGACTATCTCTAGC |
| | | CTGCAGCCCGACGACTTCGCTACCTACTACTGT |
| | | CAGCAGTACTATAACCTGCCCTGGACCTTCGGT |
| | | CAAGGCACTAAGGTCGAGATTAAGCGTACGGTG |
| | | GCCGCTCCCAGCGTGTTCATCTTCCCCCCCAGC |
| | | GACGAGCAGCTGAAGAGCGGCACCGCCAGCGTG |
| | | GTGTGCCTGCTGAACAACTTCTACCCCCGGGAG |
| | | GCCAAGGTGCAGTGGAAGGTGGACAACGCCCTG |
| | | CAGAGCGGCAACAGCCAGGAGAGCGTCACCGAG |
| | | CAGGACAGCAAGGACTCCACCTACAGCCTGAGC |
| | | AGCACCCTGACCCTGAGCAAGGCCGACTACGAG |
| | | AAGCATAAGGTGTACGCCTGCGAGGTGACCCAC |
| | | CAGGGCCTGTCCAGCCCCGTGACCAAGAGCTTC |
| | | AACAGGGGCGAGTGC |
| SEQ ID NO: 62 | DNA | GACATCCAGATGACCCAGTCCCCCTCCAGCCTG |
| | light | TCTGCTTCCGTGGGCGACAGAGTGACCATCACC |
| | chain | TGTTCCTCCAGCCAGGACATCTCCAACTACCTG |
| | | AACTGGTATCTGCAGAAGCCCGGCCAGTCCCCT |
| | | CAGCTGCTGATCTACTACACCTCCACCCTGCAC |
| | | CTGGGCGTGCCCTCCAGATTTTCCGGCTCTGGC |
| | | TCTGGCACCGAGTTTACCCTGACCATCAGCTCC |
| | | CTGCAGCCCGACGACTTCGCCACCTACTACTGC |
| | | CAGCAGTACTACAACCTGCCCTGGACCTTCGGC |
| | | CAGGGCACCAAGGTGGAAATCAAGCGTACGGTG |
| | | GCCGCTCCCAGCGTGTTCATCTTCCCCCCAAGC |
| | | GACGAGCAGCTGAAGAGCGGCACCGCCAGCGTG |
| | | GTGTGTCTGCTGAACAACTTCTACCCCAGGGAG |
| | | GCCAAGGTGCAGTGGAAGGTGGACAACGCCCTG |
| | | CAGAGCGGCAACAGCCAGGAGAGCGTCACCGAG |
| | | CAGGACAGCAAGGACTCCACCTACAGCCTGAGC |
| | | AGCACCCTGACCCTGAGCAAGGCCGACTACGAG |
| | | AAGCACAAGGTGTACGCCTGTGAGGTGACCCAC |
| | | CAGGGCCTGTCCAGCCCCGTGACCAAGAGCTTC |
| | | AACAGGGGCGAGTGC |
| BAP050-Clone J HC | | |
| SEQ ID NO: 40 | HCDR1 | NYGMN |
| (Kabat) | | |
| SEQ ID NO: 41 | HCDR2 | WINTDTGEPTYADDFKG |
| (Kabat) | | |
| SEQ ID NO: 42 | HCDR3 | NPPYYYGTNNAEAMDY |
| (Kabat) | | |
| SEQ ID NO: 43 | HCDR1 | GFTLTNY |
| (Chothia) | | |
| SEQ ID NO: 44 | HCDR2 | NTDTGE |
| (Chothia) | | |
| SEQ ID NO: 42 | HCDR3 | NPPYYYGTNNAEAMDY |
| (Chothia) | | |
| SEQ ID NO: 63 | VH | QVQLVQSGAEVKKPGASVKVSCKASGFTLTNYG |
| | | MNWVRQAPGQGLEWMGWINTDTGEPTYADDFKG |
| | | RFVFSLDTSVSTAYLQISSLKAEDTAVYYCARN |
| | | PPYYYGTNNAEAMDYWGQGTTVTVSS |
| SEQ ID NO: 64 | DNA | CAGGTGCAGCTGGTGCAGTCAGGCGCCGAAGTG |
| | VH | AAGAAACCCGGCGCTAGTGTGAAAGTCAGCTGT |
| | | AAAGCTAGTGGCTTCACCCTGACTAACTACGGG |
| | | ATGAACTGGGTCCGCCAGGCCCCAGGTCAAGGC |
| | | CTCGAGTGGATGGGCTGGATTAACACCGACACC |
| | | GGCGAGCCTACCTACGCCGACGACTTTAAGGGC |
| | | AGATTCGTGTTTAGCCTGGACACTAGTGTGTCT |
| | | ACCGCCTACCTGCAGATCTCTAGCCTGAAGGCC |
| | | GAGGACACCGCCGTCTACTACTGCGCTAGAAAC |
| | | CCCCCCTACTACTACGGCACTAACAACGCCGAG |
| | | GCTATGGACTACTGGGGTCAAGGCACTACCGTG |
| | | ACCGTGTCTAGC |
| SEQ ID NO: 65 | DNA | CAGGTGCAGCTGGTGCAGTCTGGCGCCGAAGTG |
| | VH | AAGAAACCTGGCGCCTCCGTGAAGGTGTCCTGC |
| | | AAGGCCTCTGGCTTCACCCTGACCAACTACGGC |
| | | ATGAACTGGGTGCGACAGGCCCCTGGACAGGGC |
| | | CTGGAATGGATGGGCTGGATCAACACCGACACC |
| | | GGCGAGCCTACCTACGCCGACGACTTCAAGGGC |
| | | AGATTCGTGTTCTCCCTGGACACCTCCGTGTCC |
| | | ACCGCCTACCTGCAGATCTCCAGCCTGAAGGCC |
| | | GAGGATACCGCCGTGTACTACTGCGCCCGGAAC |
| | | CCCCCTTACTACTACGGCACCAACAACGCCGAG |
| | | GCCATGGACTATTGGGGCCAGGGCACCACCGTG |
| | | ACCGTGTCCTCT |
| SEQ ID NO: 66 | Heavy | QVQLVQSGAEVKKPGASVKVSCKASGFTLTNYG |
| | chain | MNWVRQAPGQGLEWMGWINTDTGEPTYADDFKG |
| | | RFVFSLDTSVSTAYLQISSLKAEDTAVYYCARN |
| | | PPYYYGTNNAEAMDYWGQGTTVTVSSASTKGPS |
| | | VFPLAPCSRSTSESTAALGCLVKDYFPEPVTVS |
| | | WNSGALTSGVHTFPAVLQSSGLYSLSSVVTVPS |
| | | SSLGTKTYTCNVDHKPSNTKVDKRVESKYGPPC |
| | | PPCPAPEFLGGPSVFLFPPKPKDTLMISRTPEV |
| | | TCVVVDVSQEDPEVQFNWYVDGVEVHNAKTKPR |
| | | EEQFNSTYRVVSVLTVLHQDWLNGKEYKCKVSN |
| | | KGLPSSIEKTISKAKGQPREPQVYTLPPSQEEM |
| | | TKNQVSLTCLVKGFYPSDIAVEWESNGQPENNY |
| | | KTTPPVLDSDGSFFLYSRLTVDKSRWQEGNVFS |
| | | CSVMHEALHNHYTQKSLSLSLG |
| SEQ ID NO: 67 | DNA | CAGGTGCAGCTGGTGCAGTCAGGCGCCGAAGTG |
| | heavy | AAGAAACCCGGCGCTAGTGTGAAAGTCAGCTGT |
| | chain | AAAGCTAGTGGCTTCACCCTGACTAACTACGGG |
| | | ATGAACTGGGTCCGCCAGGCCCCAGGTCAAGGC |
| | | CTCGAGTGGATGGGCTGGATTAACACCGACACC |
| | | GGCGAGCCTACCTACGCCGACGACTTTAAGGGC |
| | | AGATTCGTGTTTAGCCTGGACACTAGTGTGTCT |
| | | ACCGCCTACCTGCAGATCTCTAGCCTGAAGGCC |
| | | GAGGACACCGCCGTCTACTACTGCGCTAGAAAC |
| | | CCCCCCTACTACTACGGCACTAACAACGCCGAG |
| | | GCTATGGACTACTGGGGTCAAGGCACTACCGTG |
| | | ACCGTGTCTAGCGCTAGCACTAAGGGCCCGTCC |
| | | GTGTTCCCCCTGGCACCTTGTAGCCGGAGCACT |
| | | AGCGAATCCACCGCTGCCCTCGGCTGCCTGGTC |
| | | AAGGATTACTTCCCGGAGCCCGTGACCGTGTCC |
| | | TGGAACAGCGGAGCCCTGACCTCCGGAGTGCAC |
| | | ACCTTCCCCGCTGTGCTGCAGAGCTCCGGGCTG |
| | | TACTCGCTGTCGTCGGTGGTCACGGTGCCTTCA |
| | | TCTAGCCTGGGTACCAAGACCTACACTTGCAAC |
| | | GTGGACCACAAGCCTTCCAACACTAAGGTGGAC |
| | | AAGCGCGTCGAATCGAAGTACGGCCCACCGTGC |
| | | CCGCCTTGTCCCGCGCCGGAGTTCCTCGGCGGT |
| | | CCCTCGGTCTTTCTGTTCCCACCGAAGCCCAAG |
| | | GACACTTTGATGATTTCCCGCACCCCTGAAGTG |
| | | ACATGCGTGGTCGTGGACGTGTCACAGGAAGAT |
| | | CCGGAGGTGCAGTTCAATTGGTACGTGGATGGC |
| | | GTCGAGGTGCACAACGCCAAAACCAAGCCGAGG |
| | | GAGGAGCAGTTCAACTCCACTTACCGCGTCGTG |
| | | TCCGTGCTGACGGTGCTGCATCAGGACTGGCTG |
| | | AACGGGAAGGAGTACAAGTGCAAAGTGTCCAAC |
| | | AAGGGACTTCCTAGCTCAATCGAAAAGACCATC |
| | | TCGAAAGCCAAGGGACAGCCCCGGGAACCCCAA |
| | | GTGTATACCCTGCCACCGAGCCAGGAAGAAATG |
| | | ACTAAGAACCAAGTCTCATTGACTTGCCTTGTG |
| | | AAGGGCTTCTACCCATCGGATATCGCCGTGGAA |
| | | TGGGAGTCCAACGGCCAGCCGGAAAACAACTAC |
| | | AAGACCACCCCTCCGGTGCTGGACTCAGACGGA |
| | | TCCTTCTTCCTCTACTCGCGGCTGACCGTGGAT |
| | | AAGAGCAGATGGCAGGAGGGAAATGTGTTCAGC |
| | | TGTTCTGTGATGCATGAAGCCCTGCACAACCAC |
| | | TACACTCAGAAGTCCCTGTCCCTCTCCCTGGGA |
| SEQ ID NO: 68 | DNA | CAGGTGCAGCTGGTGCAGTCTGGCGCCGAAGTG |
| | heavy | AAGAAACCTGGCGCCTCCGTGAAGGTGTCCTGC |
| | chain | AAGGCCTCTGGCTTCACCCTGACCAACTACGGC |
| | | ATGAACTGGGTGCGACAGGCCCCTGGACAGGGC |
| | | CTGGAATGGATGGGCTGGATCAACACCGACACC |
| | | GGCGAGCCTACCTACGCCGACGACTTCAAGGGC |
| | | AGATTCGTGTTCTCCCTGGACACCTCCGTGTCC |
| | | ACCGCCTACCTGCAGATCTCCAGCCTGAAGGCC |
| | | GAGGATACCGCCGTGTACTACTGCGCCCGGAAC |
| | | CCCCCTTACTACTACGGCACCAACAACGCCGAG |
| | | GCCATGGACTATTGGGGCCAGGGCACCACCGTG |
| | | ACCGTGTCCTCTGCTTCTACCAAGGGGCCCAGC |
| | | GTGTTCCCCCTGGCCCCCTGCTCCAGAAGCACC |
| | | AGCGAGAGCACAGCCGCCCTGGGCTGCCTGGTG |
| | | AAGGACTACTTCCCCGAGCCCGTGACCGTGTCC |
| | | TGGAACAGCGGAGCCCTGACCAGCGGCGTGCAC |
| | | ACCTTCCCCGCCGTGCTGCAGAGCAGCGGCCTG |
| | | TACAGCCTGAGCAGCGTGGTGACCGTGCCCAGC |
| | | AGCAGCCTGGGCACCAAGACCTACACCTGTAAC |
| | | GTGGACCACAAGCCCAGCAACACCAAGGTGGAC |
| | | AAGAGGGTGGAGAGCAAGTACGGCCCACCCTGC |
| | | CCCCCCTGCCCAGCCCCCGAGTTCCTGGGCGGA |
| | | CCCAGCGTGTTCCTGTTCCCCCCCAAGCCCAAG |
| | | GACACCCTGATGATCAGCAGAACCCCCGAGGTG |
| | | ACCTGTGTGGTGGTGGACGTGTCCCAGGAGGAC |
| | | CCCGAGGTCCAGTTCAACTGGTACGTGGACGGC |
| | | GTGGAGGTGCACAACGCCAAGACCAAGCCCAGA |
| | | GAGGAGCAGTTTAACAGCACCTACCGGGTGGTG |
| | | TCCGTGCTGACCGTGCTGCACCAGGACTGGCTG |
| | | AACGGCAAAGAGTACAAGTGTAAGGTCTCCAAC |
| | | AAGGGCCTGCCAAGCAGCATCGAAAAGACCATC |
| | | AGCAAGGCCAAGGGCCAGCCTAGAGAGCCCCAG |
| | | GTCTACACCCTGCCACCCAGCCAAGAGGAGATG |
| | | ACCAAGAACCAGGTGTCCCTGACCTGTCTGGTG |
| | | AAGGGCTTCTACCCAAGCGACATCGCCGTGGAG |
| | | TGGGAGAGCAACGGCCAGCCCGAGAACAACTAC |
| | | AAGACCACCCCCCCAGTGCTGGACAGCGACGGC |
| | | AGCTTCTTCCTGTACAGCAGGCTGACCGTGGAC |
| | | AAGTCCAGATGGCAGGAGGGCAACGTCTTTAGC |
| | | TGCTCCGTGATGCACGAGGCCCTGCACAACCAC |
| | | TACACCCAGAAGAGCCTGAGCCTGTCCCTGGGC |
| BAP050-Clone J LC | | |
| SEQ ID NO: 49 | LCDR1 | SSSQDISNYLN |
| (Kabat) | | |
| SEQ ID NO: 50 | LCDR2 | YTSTLHL |
| (Kabat) | | |
| SEQ ID NO: 51 | LCDR3 | QQYYNLPWT |
| (Kabat) | | |
| SEQ ID NO: 52 | LCDR1 | SQDISNY |
| (Chothia) | | |
| SEQ ID NO: 53 | LCDR2 | YTS |
| (Chothia) | | |
| SEQ ID NO: 54 | LCDR3 | YYNLPW |
| (Chothia) | | |
| SEQ ID NO: 69 | VL | DIQMTQSPSSLSASVGDRVTITCSSSQDISNYL |
| | | NWYQQKPGKAPKLLIYYTSTLHLGIPPRFSGSG |
| | | YGTDFTLTINNIESEDAAYYFCQQYYNLPWTFG |
| | | QGTKVEIK |
| SEQ ID NO: 70 | DNA | GATATTCAGATGACTCAGTCACCTAGTAGCCTG |
| | VL | AGCGCTAGTGTGGGCGATAGAGTGACTATCACC |
| | | TGTAGCTCTAGTCAGGATATCTCTAACTACCTG |
| | | AACTGGTATCAGCAGAAGCCCGGTAAAGCCCCT |
| | | AAGCTGCTGATCTACTACACTAGCACCCTGCAC |
| | | CTGGGAATCCCCCCTAGGTTTAGCGGTAGCGGC |
| | | TACGGCACCGACTTCACCCTGACTATTAACAAT |
| | | ATCGAGTCAGAGGACGCCGCCTACTACTTCTGT |
| | | CAGCAGTACTATAACCTGCCCTGGACCTTCGGT |
| | | CAAGGCACTAAGGTCGAGATTAAG |
| SEQ ID NO: 71 | DNA | GACATCCAGATGACCCAGTCCCCCTCCAGCCTG |
| | VL | TCTGCTTCCGTGGGCGACAGAGTGACCATCACC |
| | | TGTTCCTCCAGCCAGGACATCTCCAACTACCTG |
| | | AACTGGTATCAGCAGAAGCCCGGCAAGGCCCCC |
| | | AAGCTGCTGATCTACTACACCTCCACCCTGCAC |
| | | CTGGGCATCCCCCCTAGATTCTCCGGCTCTGGC |
| | | TACGGCACCGACTTCACCCTGACCATCAACAAC |
| | | ATCGAGTCCGAGGACGCCGCCTACTACTTCTGC |
| | | CAGCAGTACTACAACCTGCCCTGGACCTTCGGC |
| | | CAGGGCACCAAGGTGGAAATCAAG |
| SEQ ID NO: 72 | Light | DIQMTQSPSSLSASVGDRVTITCSSSQDISNYL |
| | chain | NWYQQKPGKAPKLLIYYTSTLHLGIPPRFSGSG |
| | | YGTDFTLTINNIESEDAAYYFCQQYYNLPWTFG |
| | | QGTKVEIKRTVAAPSVFIFPPSDEQLKSGTASV |
| | | VCLLNNFYPREAKVQWKVDNALQSGNSQESVTE |
| | | QDSKDSTYSLSSTLTLSKADYEKHKVYACEVTH |
| | | QGLSSPVTKSFNRGEC |
| SEQ ID NO: 73 | DNA | GATATTCAGATGACTCAGTCACCTAGTAGCCTG |
| | light | AGCGCTAGTGTGGGCGATAGAGTGACTATCACC |
| | chain | TGTAGCTCTAGTCAGGATATCTCTAACTACCTG |
| | | AACTGGTATCAGCAGAAGCCCGGTAAAGCCCCT |
| | | AAGCTGCTGATCTACTACACTAGCACCCTGCAC |
| | | CTGGGAATCCCCCCTAGGTTTAGCGGTAGCGGC |
| | | TACGGCACCGACTTCACCCTGACTATTAACAAT |
| | | ATCGAGTCAGAGGACGCCGCCTACTACTTCTGT |
| | | CAGCAGTACTATAACCTGCCCTGGACCTTCGGT |
| | | CAAGGCACTAAGGTCGAGATTAAGCGTACGGTG |
| | | GCCGCTCCCAGCGTGTTCATCTTCCCCCCCAGC |
| | | GACGAGCAGCTGAAGAGCGGCACCGCCAGCGTG |
| | | GTGTGCCTGCTGAACAACTTCTACCCCCGGGAG |
| | | GCCAAGGTGCAGTGGAAGGTGGACAACGCCCTG |
| | | CAGAGCGGCAACAGCCAGGAGAGCGTCACCGAG |
| | | CAGGACAGCAAGGACTCCACCTACAGCCTGAGC |
| | | AGCACCCTGACCCTGAGCAAGGCCGACTACGAG |
| | | AAGCATAAGGTGTACGCCTGCGAGGTGACCCAC |
| | | CAGGGCCTGTCCAGCCCCGTGACCAAGAGCTTC |
| | | AACAGGGGCGAGTGC |
| SEQ ID NO: 74 | DNA | GACATCCAGATGACCCAGTCCCCCTCCAGCCTG |
| | light | TCTGCTTCCGTGGGCGACAGAGTGACCATCACC |
| | chain | TGTTCCTCCAGCCAGGACATCTCCAACTACCTG |
| | | AACTGGTATCAGCAGAAGCCCGGCAAGGCCCCC |
| | | AAGCTGCTGATCTACTACACCTCCACCCTGCAC |
| | | CTGGGCATCCCCCCTAGATTCTCCGGCTCTGGC |
| | | TACGGCACCGACTTCACCCTGACCATCAACAAC |
| | | ATCGAGTCCGAGGACGCCGCCTACTACTTCTGC |
| | | CAGCAGTACTACAACCTGCCCTGGACCTTCGGC |
| | | CAGGGCACCAAGGTGGAAATCAAGCGTACGGTG |
| | | GCCGCTCCCAGCGTGTTCATCTTCCCCCCAAGC |
| | | GACGAGCAGCTGAAGAGCGGCACCGCCAGCGTG |
| | | GTGTGTCTGCTGAACAACTTCTACCCCAGGGAG |
| | | GCCAAGGTGCAGTGGAAGGTGGACAACGCCCTG |
| | | CAGAGCGGCAACAGCCAGGAGAGCGTCACCGAG |
| | | CAGGACAGCAAGGACTCCACCTACAGCCTGAGC |
| | | AGCACCCTGACCCTGAGCAAGGCCGACTACGAG |
| | | AAGCACAAGGTGTACGCCTGTGAGGTGACCCAC |
| | | CAGGGCCTGTCCAGCCCCGTGACCAAGAGCTTC |
| | | AACAGGGGCGAGTGC |
| BAP050-Clone I HC | | |
| SEQ ID NO: 75 | HCDR1 | AATTACGGGATGAAC |
| (Kabat) | | |
| SEQ ID NO: 76 | HCDR1 | AACTACGGCATGAAC |
| (Kabat) | | |
| SEQ ID NO: 77 | HCDR2 | TGGATTAACACCGACACCGGGGAGCCTACCTAC |
| (Kabat) | | GCGGACGATTTCAAGGGA |
| SEQ ID NO: 78 | HCDR2 | TGGATCAACACCGACACCGGCGAGCCTACCTAC |
| (Kabat) | | GCCGACGACTTCAAGGGC |
| SEQ ID NO: 79 | HCDR3 | AACCCGCCCTACTACTACGGAACCAACAACGCC |
| (Kabat) | | GAAGCCATGGACTAC |
| SEQ ID NO: 80 | HCDR3 | AACCCCCCTTACTACTACGGCACCAACAACGCC |
| (Kabat) | | GAGGCCATGGACTAT |
| SEQ ID NO: 81 | HCDR1 | GGATTCACCCTCACCAATTAC |
| (Chothia) | | |
| SEQ ID NO: 82 | HCDR1 | GGCTTCACCCTGACCAACTAC |
| (Chothia) | | |
| SEQ ID NO: 83 | HCDR2 | AACACCGACACCGGGGAG |
| (Chothia) | | |
| SEQ ID NO: 84 | HCDR2 | AACACCGACACCGGCGAG |
| (Chothia) | | |
| SEQ ID NO: 79 | HCDR3 | AACCCGCCCTACTACTACGGAACCAACAACGCC |
| (Chothia) | | GAAGCCATGGACTAC |
| SEQ ID NO: 80 | HCDR3 | AACCCCCCTTACTACTACGGCACCAACAACGCC |
| (Chothia) | | GAGGCCATGGACTAT |
| BAP050-Clone ILC | | |
| SEQ ID NO: 85 | LCDR1 | AGCTCTAGTCAGGATATCTCTAACTACCTGAAC |
| (Kabat) | | |
| SEQ ID NO: 86 | LCDR1 | TCCTCCAGCCAGGACATCTCCAACTACCTGAAC |
| (Kabat) | | |
| SEQ ID NO: 87 | LCDR2 | TACACTAGCACCCTGCACCTG |
| (Kabat) | | |
| SEQ ID NO: 88 | LCDR2 | TACACCTCCACCCTGCACCTG |
| (Kabat) | | |
| SEQ ID NO: 89 | LCDR3 | CAGCAGTACTATAACCTGCCCTGGACC |
| (Kabat) | | |
| SEQ ID NO: 90 | LCDR3 | CAGCAGTACTACAACCTGCCCTGGACC |
| (Kabat) | | |
| SEQ ID NO: 91 | LCDR1 | AGTCAGGATATCTCTAACTAC |
| (Chothia) | | |
| SEQ ID NO: 92 | LCDR1 | AGCCAGGACATCTCCAACTAC |
| (Chothia) | | |
| SEQ ID NO: 93 | LCDR2 | TACACTAGC |
| (Chothia) | | |
| SEQ ID NO: 94 | LCDR2 | TACACCTCC |
| (Chothia) | | |
| SEQ ID NO: 95 | LCDR3 | TACTATAACCTGCCCTGG |
| (Chothia) | | |
| SEQ ID NO: 96 | LCDR3 | TACTACAACCTGCCCTGG |
| (Chothia) | | |
| BAP050-Clone J HC | | |
| SEQ ID NO: 97 | HCDR1 | AACTACGGGATGAAC |
| (Kabat) | | |
| SEQ ID NO: 76 | HCDR1 | AACTACGGCATGAAC |
| (Kabat) | | |
| SEQ ID NO: 98 | HCDR2 | TGGATTAACACCGACACCGGCGAGCCTACCTAC |
| (Kabat) | | GCCGACGACTTTAAGGGC |
| SEQ ID NO: 78 | HCDR2 | TGGATCAACACCGACACCGGCGAGCCTACCTAC |
| (Kabat) | | GCCGACGACTTCAAGGGC |
| SEQ ID NO: 99 | HCDR3 | AACCCCCCCTACTACTACGGCACTAACAACGCC |
| (Kabat) | | GAGGCTATGGACTAC |
| SEQ ID NO: 80 | HCDR3 | AACCCCCCTTACTACTACGGCACCAACAACGCC |
| (Kabat) | | GAGGCCATGGACTAT |
| SEQ ID NO: 100 | HCDR1 | GGCTTCACCCTGACTAACTAC |
| (Chothia) | | |
| SEQ ID NO: 82 | HCDR1 | GGCTTCACCCTGACCAACTAC |
| (Chothia) | | |
| SEQ ID NO: 83 | HCDR2 | AACACCGACACCGGGGAG |
| (Chothia) | | |
| SEQ ID NO: 84 | HCDR2 | AACACCGACACCGGCGAG |
| (Chothia) | | |
| SEQ ID NO: 99 | HCDR3 | AACCCCCCCTACTACTACGGCACTAACAACGCC |
| (Chothia) | | GAGGCTATGGACTAC |
| SEQ ID NO: 80 | HCDR3 | AACCCCCCTTACTACTACGGCACCAACAACGCC |
| (Chothia) | | GAGGCCATGGACTAT |
| BAP050-Clone J LC | | |
| SEQ ID NO: 85 | LCDR1 | AGCTCTAGTCAGGATATCTCTAACTACCTGAAC |
| (Kabat) | | |
| SEQ ID NO: 86 | LCDR1 | TCCTCCAGCCAGGACATCTCCAACTACCTGAAC |
| (Kabat) | | |
| SEQ ID NO: 87 | LCDR2 | TACACTAGCACCCTGCACCTG |
| (Kabat) | | |
| SEQ ID NO: 88 | LCDR2 | TACACCTCCACCCTGCACCTG |
| (Kabat) | | |
| SEQ ID NO: 89 | LCDR3 | CAGCAGTACTATAACCTGCCCTGGACC |
| (Kabat) | | |
| SEQ ID NO: 90 | LCDR3 | CAGCAGTACTACAACCTGCCCTGGACC |
| (Kabat) | | |
| SEQ ID NO: 91 | LCDR1 | AGTCAGGATATCTCTAACTAC |
| (Chothia) | | |
| SEQ ID NO: 92 | LCDR1 | AGCCAGGACATCTCCAACTAC |
| (Chothia) | | |
| SEQ ID NO: 93 | LCDR2 | TACACTAGC |
| (Chothia) | | |
| SEQ ID NO: 94 | LCDR2 | TACACCTCC |
| (Chothia) | | |
| SEQ ID NO: 95 | LCDR3 | TACTATAACCTGCCCTGG |
| (Chothia) | | |
| SEQ ID NO: 96 | LCDR3 | TACTACAACCTGCCCTGG |
| (Chothia) | | |

In some embodiments, the LAG-3 inhibitor (e.g., an anti-LAG-3 antibody molecule described herein) is administered at a dose of about 300-1000 mg, e.g., about 300 mg to about 500 mg, about 400 mg to about 800 mg, or about 700 mg to about 900 mg. In embodiments, the LAG-3 inhibitor is administered once every week, once every two weeks, once every three weeks, once every four weeks, once every five weeks or once every six weeks. In embodiments, the LAG-3 inhibitor is administered once every 3 weeks. In embodiments, the LAG-3 inhibitor is administered once every 4 weeks. In other embodiments, the LAG-3 inhibitor is administered at a dose of about 300 mg to about 500 mg (e.g., about 400 mg) once every 3 weeks. In yet other embodiments, the LAG-3 inhibitor is administered at a dose of about 700 mg to about 900 mg (e.g., about 800 mg) once every 4 weeks. In yet other embodiments, the LAG-3 inhibitor is administered at a dose of about 400 mg to about 800 mg (e.g., about 600 mg) once every 4 weeks.

In some embodiments, a composition or method comprises a LAG-3 inhibitor, e.g., a LAG-3 inhibitor described herein, and a PD-1 inhibitor, e.g., a PD-1 inhibitor described herein. In some embodiments, the combination of a LAG-3 inhibitor and a PD-1 inhibitor is administered in a therapeutically effective amount to a subject with a solid tumor, e.g., a prostate cancer. Without wishing to be bound by theory, it is believed that a combination comprising a LAG-3 inhibitor and a PD-1 inhibitor has increased activity compared to administration of a PD-1 inhibitor alone.

In some embodiments, a composition or method comprises a LAG-3 inhibitor, e.g., a LAG-3 inhibitor described herein, a GITR agonist, e.g., a GITR agonist described herein, and a PD-1 inhibitor, e.g., a PD-1 inhibitor described herein. In some embodiments, the combination of a LAG-3 inhibitor, a GITR agonist, and a PD-1 inhibitor is administered in a therapeutically effective amount to a subject with a solid tumor, e.g., a prostate cancer. In some embodiments, a combination comprising a LAG-3 inhibitor, a GITR agonist, and a PD-1 inhibitor can result in increased IL-2 production.

### Other Exemplary LAG-3 Inhibitors

In one embodiment, the anti-LAG-3 antibody molecule is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated herein by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of BMS-986016, e.g., as disclosed in Table 4.

In one embodiment, the anti-LAG-3 antibody molecule is TSR-033 (Tesaro). In one embodiment, the anti-LAG-3 antibody molecule comprises one or more of the CDR sequences (or collectively all of the CDR sequences), the heavy chain or light chain variable region sequence, or the heavy chain or light chain sequence of TSR-033.

In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated herein by reference in their entirety. In one embodiment, the anti-LAG-3 antibody molecule comprises one or more of the CDR sequences (or collectively all of the CDR sequences), the heavy chain or light chain variable region sequence, or the heavy chain or light chain sequence of IMP731, e.g., as disclosed in Table 4. In one embodiment, the anti-LAG-3 antibody molecule comprises one or more of the CDR sequences (or collectively all of the CDR sequences), the heavy chain or light chain variable region sequence, or the heavy chain or light chain sequence of GSK2831781.

In one embodiment, the anti-LAG-3 antibody molecule is IMP761 (Prima BioMed). In one embodiment, the anti-LAG-3 antibody molecule comprises one or more of the CDR sequences (or collectively all of the CDR sequences), the heavy chain or light chain variable region sequence, or the heavy chain or light chain sequence of IMP761.

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated herein by reference in their entirety.

In one embodiment, the anti-LAG-3 antibody is an antibody that competes for binding with, and/or binds to the same epitope on LAG-3 as, one of the anti-LAG-3 antibodies described herein.

In one embodiment, the anti-LAG-3 inhibitor is a soluble LAG-3 protein, e.g., IMP321 (Prima BioMed), e.g., as disclosed in WO 2009/044273, incorporated herein by reference in its entirety.

TABLE 4
Amino acid sequences of other exemplary anti-LAG-3
antibody molecules

| | | |
|---|---|---|
| BMS-986016 | | |
| SEQ ID NO: | Heavy | QVQLQQWGAGLLKPSETLSLTCAVYGGSFSDYYWN |
| 101 | chain | WIRQPPGKGLEWIGEINHRGSTNSNPSLKSRVTLSLDT |
| | | SKNQFSLKLRSVTAADTAVYYCAFGYSDYEYNWFDP |
| | | WGQGTLVTVSSASTKGPSVFPLAPCSRSTSESTAALG |
| | | CLVKDYFPEPVTVSWNSGALTSGVHTFPAVLQSSGL |
| | | YSLSSVVTVPSSSLGTKTYTCNVDHKPSNTKVDKRV |
| | | ESKYGPPCPPCPAPEFLGGPSVFLFPPKPKDTLMISRTP |
| | | EVTCVVVDVSQEDPEVQFNWYVDGVEVHNAKTKPR |
| | | EEQFNSTYRVVSVLTVLHQDWLNGKEYKCKVSNKG |
| | | LPSSIEKTISKAKGQPREPQVYTLPPSQEEMTKNQVSL |
| | | TCLVKGFYPSDIAVEWESNGQPENNYKTTPPVLDSD |
| | | GSFFLYSRLTVDKSRWQEGNVFSCSVMHEALHNHYT |
| | | QKSLSLSLGK |
| SEQ ID NO: | Light | EIVLTQSPATLSLSPGERATLSCRASQSISSYLAWYQQ |
| 102 | chain | KPGQAPRLLIYDASNRATGIPARFSGSGSGTDFTLTIS |
| | | SLEPEDFAVYYCQQRSNWPLTFGQGTNLEIKRTVAAP |
| | | SVFIFPPSDEQLKSGTASVVCLLNNFYPREAKVQWKV |
| | | DNALQSGNSQESVTEQDSKDSTYSLSSTLTLSKADYE |
| | | KHKVYACEVTHQGLSSPVTKSFNRGEC |
| IMP731 | | |
| SEQ ID NO: | Heavy | QVQLKESGPGLVAPSQSLSITCTVSGFSLTAYGVNWV |
| 103 | chain | RQPPGKGLEWLGMIWDDGSTDYNSALKSRLSISKDN |
| | | SKSQVFLKMNSLQTDDTARYYCAREGDVAFDYWGQ |
| | | GTTLTVSSASTKGPSVFPLAPSSKSTSGGTAALGCLV |
| | | KDYFPEPVTVSWNSGALTSGVHTFPAVLQSSGLYSLS |
| | | SVVTVPSSSLGTQTYICNVNHKPSNTKVDKKVEPKSC |
| | | DKTHTCPPCPAPELLGGPSVFLFPPKPKDTLMISRTPE |
| | | VTCVVVDVSHEDPEVKFNWYVDGVEVHNAKTKPRE |
| | | EQYNSTYRVVSVLTVLHQDWLNGKEYKCKVSNKAL |
| | | PAPIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEALHNHYTQ |
| | | KSLSLSPGK |
| SEQ ID NO: | Light | DIVMTQSPSSLAVSVGQKVTMSCKSSQSLLNGSNQK |
| 104 | chain | NYLAWYQQKPGQSPKLLVYFASTRDSGVPDRFIGSG |
| | | SGTDFTLTISSVQAEDLADYFCLQHFGTPPTFGGGTK |
| | | LEIKRTVAAPSVFIFPPSDEQLKSGTASVVCLLNNFYP |
| | | REAKVQWKVDNALQSGNSQESVTEQDSKDSTYSLSS |
| | | TLTLSKADYEKHKVYACEVTHQGLSSPVTKSFNRGE |
| | | C |

### TIM-3 Inhibitors

In certain embodiments, the combination according to the invention comprising a PSMA therapeutic agent or method described herein comprises a TIM-3 inhibitor as I-O therapeutic agent. Without wishing to be bound by theory, it is believed that TIM-3 correlates with tumor myeloid signature in The Cancer Genome Atlas (TCGA) database and the most abundant TIM-3 on normal peripheral blood mononuclear cells (PBMCs) is on myeloid cells. TIM-3 is expressed on multiple myeloid subsets in human PBMCs, including, but not limited to, monocytes, macrophages and dendritic cells.

Tumor purity estimates are negatively correlated with TIM-3 expression in a number of TCGA tumor samples (including, e.g., adrenocortical carcinoma (ACC), bladder urothelial carcinoma (BLCA), breast invasive carcinoma (BRCA), cervical squamous cell carcinoma and endocervical adenocarcinoma (CESC), colon adenocarcinoma (COAD), glioblastoma multiforme (GBM), head and neck squamous cell carcinoma (HNSC), kidney chromophobe (KICH), kidney renal clear cell carcinoma (KIRC), kidney renal papillary cell carcinoma (KIRP), brain low grade glioma (LGG), liver hepatocellular carcinoma (LIHC), lung adenocarcinoma (LUAD), lung squamous cell carcinoma (LUSC), ovarian serous cystadenocarcinoma (OV), prostate adenocarcinoma (PRAD), rectum adenocarcinoma (READ), skin cutaneous melanoma (SKCM), thyroid carcinoma (THCA), uterine corpus endometrial carcinoma (UCEC), and uterine carcinosarcoma (UCS)), suggesting TIM-3 expression in tumor samples is from tumor infiltrates.

In certain embodiments, the combination or method is used to treat a kidney cancer (e.g., a kidney renal clear cell carcinoma (KIRC) or a kidney renal papillary cell carcinoma (KIRP)). In other embodiments, the combination is used to treat a brain tumor (e.g., a brain low grade glioma (LGG) or a glioblastoma multiforme (GBM)). In some embodiments, the combination is used to treat a mesothelioma (MESO). In some embodiments, the combination is used to treat a sarcoma (SARC), a lung adenocarcinoma (LUAD), a pancreatic adenocarcinoma (PAAD), a lung squamous cell carcinoma (LUSC), or a prostate cancer.

Without wishing to be bound by theory, it is believed that in some embodiments, by clustering indications by immune signatures, cancers that can be effectively treated by a combination or method described herein can be identified, e.g., by determining the fraction of patients in each indication above 75^{th} percentile across TCGA.

In some embodiments, a T cell gene signature comprises expression of one or more (e.g., all) of: CD2, CD247, CD3D, CD3E, CD3G, CD8A, CD8B, CXCR6, GZMK, PYHIN1, SH2D1A, SIRPG or TRAT1.

In some embodiments, a Myeloid gene signature comprises expression of one or more (e.g., all) of SIGLEC1, MSR1, LILRB4, ITGAM or CD163.

In some embodiments, a TIM-3 gene signature comprises expression of one or more (e.g., all) of HAVCR2, ADGRG1, PIK3AP1, CCL3, CCL4, PRF1, CD8A, NKG7, or KLRK1.

Without wishing to be bound by theory, it is believed that in some embodiments, a TIM-3 inhibitor, e.g., MBG453, may synergize with a PD-1 inhibitor, e.g., PDR001, in a mixed lymphocyte reaction (MLR) assay. In some embodiments, inhibition of PD-L1 and TIM-3 may result in tumor reduction and survival in mouse models of cancer. In some embodiments, inhibition of PD-L1 and LAG-3 may result in tumor reduction and survival in mouse models of cancer.

In some embodiments, the combination comprising PSMA therapeutic agent and additional therapeutic agent is used to treat a cancer having high levels of expression of TIM-3 and one or more of myeloid signature genes (e.g., one or more genes expressed in macrophages). In some embodiments, the cancer having high levels of expression of TIM-3 and myeloid signature genes is chosen from a sarcoma (SARC), a mesothelioma (MESO), a brain tumor (e.g., a glioblastoma (GBM), or a kidney cancer (e.g., a kidney renal papillary cell carcinoma (KIRP)), or a prostate cancer. In other embodiments, the combination or method is used to treat a cancer having high levels of expression of TIM-3 and one or more of T cell signature genes (e.g., one or more genes expressed in dendritic cells and/or T cells). In some embodiments, the cancer having high levels of expression of TIM-3 and T cell signature genes is chosen from a kidney cancer (e.g., a kidney renal clear cell carcinoma (KIRC)), a lung cancer (e.g., a lung adenocarcinoma (LUAD)), a pancreatic adenocarcinoma (PAAD), a prostate cancer, or a testicular cancer (e.g., a testicular germ cell tumor (TGCT)).

Without wishing to be bound by theory, it is believed that in some embodiments, by clustering indications by immune signatures, cancers that can be effectively treated by a combination or method targeting two, three, or more targets described herein can be identified, e.g., by determining the fraction of patients above 75^{th} percentile in both or all of the targets.

In some embodiments, the combination or method according to the invention comprises a TIM-3 inhibitor (e.g., a TIM-3 inhibitor described herein) and a PD-1 inhibitor (e.g., a PD-1 inhibitor described herein) as I-O therapeutic agents, e.g., to treat cancer chosen from a kidney cancer (e.g., a kidney renal papillary cell carcinoma (KIRC) or a kidney renal papillary cell carcinoma (KIRP)), a mesothelioma (MESO), a lung cancer (e.g., a lung adenocarcinoma (LUAD) or a lung squamous cell carcinoma (LUSC)), a sarcoma (SARC), a testicular cancer (e.g., a testicular germ cell tumor (TGCT)), a prostate cancer, a pancreatic cancer (e.g., a pancreatic adenocarcinoma (PAAD)), a cervical cancer (e.g., cervical squamous cell carcinoma and endocervical adenocarcinoma (CESC)), a head and neck cancer (e.g., a head and neck squamous cell carcinoma (HNSC)), a bladder cancer (e.g., bladder urothelial carcinoma (BLCA), a stomach cancer (e.g., stomach adenocarcinoma (STAD)), a skin cancer (e.g., skin cutaneous melanoma (SKCM)), a breast cancer (e.g., breast invasive carcinoma (BRCA)), or a cholangiocarcinoma (CHOL).

In some embodiments, the combination or method according to the invention comprises a TIM-3 inhibitor (e.g., a TIM-3 inhibitor described herein) and a PD-1 inhibitor (e.g., a PD-1 inhibitor described herein) as I-O therapeutic agents to treat PSMA expressing cancer.

In some embodiments, the combination or method according to the invention comprises a TIM-3 inhibitor (e.g., a TIM-3 inhibitor described herein) and a LAG-3 inhibitor (e.g., a LAG-3 inhibitor described herein) as I-O therapeutic agents, e.g., to treat cancer chosen from a kidney cancer (e.g., a kidney renal papillary cell carcinoma (KIRC)), a mesothelioma (MESO), a lung cancer (e.g., a lung adenocarcinoma (LUAD) or a lung squamous cell carcinoma (LUSC)), a sarcoma (SARC), a testicular cancer (e.g., a testicular germ cell tumor (TGCT)), a cervical cancer (e.g., cervical squamous cell carcinoma and endocervical adenocarcinoma (CESC)), an ovarian cancer (OV), a head and neck cancer (e.g., a head and neck squamous cell carcinoma (HNSC)), a stomach cancer (e.g., stomach adenocarcinoma (STAD)), a bladder cancer (e.g., bladder urothelial carcinoma (BLCA), a breast cancer (e.g., breast invasive carcinoma (BRCA)), a prostate cancer, or a skin cancer (e.g., skin cutaneous melanoma (SKCM)).

In some embodiments, the combination or method according to the invention comprises a TIM-3 inhibitor (e.g., a TIM-3 inhibitor described herein) and a LAG-3 inhibitor (e.g., a LAG-3 inhibitor described herein) as I-O therapeutic agents to treat PSMA expressing cancer.

In some embodiments, the combination or method according to the invention comprises a TIM-3 inhibitor (e.g., a TIM-3 inhibitor described herein), a PD-1 inhibitor (e.g., a PD-1 inhibitor described herein), and a LAG-3 inhibitor (e.g., a LAG-3 inhibitor described herein) as I-O therapeutic agents, e.g., to treat a cancer chosen from a kidney cancer (e.g., a kidney renal papillary cell carcinoma (KIRC)), a lung cancer (e.g., a lung adenocarcinoma (LUAD) or a lung squamous cell carcinoma (LUSC)), a mesothelioma (MESO), a testicular cancer (e.g., a testicular germ cell tumor (TGCT)), a sarcoma (SARC), a cervical cancer (e.g., cervical squamous cell carcinoma and endocervical adenocarcinoma (CESC)), a head and neck cancer (e.g., a head and neck squamous cell carcinoma (HNSC)), a stomach cancer (e.g., stomach adenocarcinoma (STAD)), an ovarian cancer (OV), a bladder cancer (e.g., bladder urothelial carcinoma (BLCA), a breast cancer (e.g., breast invasive carcinoma (BRCA)), a prostate cancer, or a skin cancer (e.g., skin cutaneous melanoma (SKCM)).

In some embodiments, the combination or method according to the invention comprises a TIM-3 inhibitor (e.g., a TIM-3 inhibitor described herein), a PD-1 inhibitor (e.g., a PD-1 inhibitor described herein), and a LAG-3 inhibitor (e.g., a LAG-3 inhibitor described herein) as I-O therapeutic agents to treat PSMA expressing cancer.

In some embodiments, the combination or method according to the invention comprises a TIM-3 inhibitor (e.g., a TIM-3 inhibitor described herein), a PD-1 inhibitor (e.g., a PD-1 inhibitor described herein), and a c-MET inhibitor (e.g., a c-MET inhibitor described herein), e.g., to treat a cancer chosen from a kidney cancer (e.g., a kidney renal papillary cell carcinoma (KIRC)), a lung cancer (e.g., a lung adenocarcinoma (LUAD), a prostate cancer, or a mesothelioma (MESO).

In some embodiments, the combination or method according to the invention comprises a TIM-3 inhibitor (e.g., a TIM-3 inhibitor described herein), a PD-1 inhibitor (e.g., a PD-1 inhibitor described herein), and a c-MET inhibitor (e.g., a c-MET inhibitor described herein) as I-O therapeutic agent to treat PSMA expressing cancer.

In some embodiments, the TIM-3 inhibitor is MBG453 (Novartis) or TSR-022 (Tesaro). In some embodiments, the TIM-3 inhibitor is MBG453.

### Exemplary TIM-3 Inhibitors

In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated herein by reference in its entirety.

In one embodiment, the anti-TIM-3 antibody molecule comprises at least one, two, three, four, five or six complementarity determining regions (CDRs) (or collectively all of the CDRs) from a heavy and light chain variable region comprising an amino acid sequence shown in Table 5 (e.g., from the heavy and light chain variable region sequences of ABTIM3-hum11 or ABTIM3-hum03 disclosed in Table 5), or encoded by a nucleotide sequence shown in Table 5. In some embodiments, the CDRs are according to the Kabat definition (e.g., as set out in Table 5). In some embodiments, the CDRs are according to the Chothia definition (e.g., as set out in Table 5). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 5, or encoded by a nucleotide sequence shown in Table 5.

In one embodiment, the anti-TIM-3 antibody molecule comprises a heavy chain variable region (VH) comprising a VHCDR1 amino acid sequence of SEQ ID NO: 106, a VHCDR2 amino acid sequence of SEQ ID NO: 107, and a VHCDR3 amino acid sequence of SEQ ID NO: 108; and a light chain variable region (VL) comprising a VLCDR1 amino acid sequence of SEQ ID NO: 115, a VLCDR2 amino acid sequence of SEQ ID NO: 116, and a VLCDR3 amino acid sequence of SEQ ID NO: 117, each disclosed in Table 5. In one embodiment, the anti-TIM-3 antibody molecule comprises a heavy chain variable region (VH) comprising a VHCDR1 amino acid sequence of SEQ ID NO: 106, a VHCDR2 amino acid sequence of SEQ ID NO: 125, and a VHCDR3 amino acid sequence of SEQ ID NO: 108; and a light chain variable region (VL) comprising a VLCDR1 amino acid sequence of SEQ ID NO: 115, a VLCDR2 amino acid sequence of SEQ ID NO: 116, and a VLCDR3 amino acid sequence of SEQ ID NO: 117, each disclosed in Table 5.

In one embodiment, the anti-TIM-3 antibody molecule comprises a VH comprising the amino acid sequence of SEQ ID NO: 111, or an amino acid sequence at least 85%, 90%, 95%, 96%, 97%, 98%, or 99% identical or higher to SEQ ID NO: 111. In one embodiment, the anti-TIM-3 antibody molecule comprises a VL comprising the amino acid sequence of SEQ ID NO: 121, or an amino acid sequence at least 85%, 90%, 95%, 96%, 97%, 98%, or 99% identical or higher to SEQ ID NO: 121. In one embodiment, the anti-TIM-3 antibody molecule comprises a VH comprising the amino acid sequence of SEQ ID NO: 127, or an amino acid sequence at least 85%, 90%, 95%, 96%, 97%, 98%, or 99% identical or higher to SEQ ID NO: 127. In one embodiment, the anti-TIM-3 antibody molecule comprises a VL comprising the amino acid sequence of SEQ ID NO: 131, or an amino acid sequence at least 85%, 90%, 95%, 96%, 97%, 98%, or 99% identical or higher to SEQ ID NO: 131. In one embodiment, the anti-TIM-3 antibody molecule comprises a VH comprising the amino acid sequence of SEQ ID NO: 111 and a VL comprising the amino acid sequence of SEQ ID NO: 121. In one embodiment, the anti-TIM-3 antibody molecule comprises a VH comprising the amino acid sequence of SEQ ID NO: 127 and a VL comprising the amino acid sequence of SEQ ID NO: 128.

In one embodiment, the antibody molecule comprises a VH encoded by the nucleotide sequence of SEQ ID NO: 112, or a nucleotide sequence at least 85%, 90%, 95%, 96%, 97%, 98%, or 99% identical or higher to SEQ ID NO: 112. In one embodiment, the antibody molecule comprises a VL encoded by the nucleotide sequence of SEQ ID NO: 122, or a nucleotide sequence at least 85%, 90%, 95%, 96%, 97%, 98%, or 99% identical or higher to SEQ ID NO: 122. In one embodiment, the antibody molecule comprises a VH encoded by the nucleotide sequence of SEQ ID NO: 128, or a nucleotide sequence at least 85%, 90%, 95%, 96%, 97%, 98%, or 99% identical or higher to SEQ ID NO: 128. In one embodiment, the antibody molecule comprises a VL encoded by the nucleotide sequence of SEQ ID NO: 132, or a nucleotide sequence at least 85%, 90%, 95%, 96%, 97%, 98%, or 99% identical or higher to SEQ ID NO: 132. In one embodiment, the antibody molecule comprises a VH encoded by the nucleotide sequence of SEQ ID NO: 112 and a VL encoded by the nucleotide sequence of SEQ ID NO: 122. In one embodiment, the antibody molecule comprises a VH encoded by the nucleotide sequence of SEQ ID NO: 128 and a VL encoded by the nucleotide sequence of SEQ ID NO: 132.

In one embodiment, the anti-TIM-3 antibody molecule comprises a heavy chain comprising the amino acid sequence of SEQ ID NO: 113, or an amino acid sequence at least 85%, 90%, 95%, 96%, 97%, 98%, or 99% identical or higher to SEQ ID NO: 113. In one embodiment, the anti-TIM-3 antibody molecule comprises a light chain comprising the amino acid sequence of SEQ ID NO: 123, or an amino acid sequence at least 85%, 90%, 95%, 96%, 97%, 98%, or 99% identical or higher to SEQ ID NO: 123. In one embodiment, the anti-TIM-3 antibody molecule comprises a heavy chain comprising the amino acid sequence of SEQ ID NO: 129, or an amino acid sequence at least 85%, 90%, 95%, 96%, 97%, 98%, or 99% identical or higher to SEQ ID NO: 129. In one embodiment, the anti-TIM-3 antibody molecule comprises a light chain comprising the amino acid sequence of SEQ ID NO: 133, or an amino acid sequence at least 85%, 90%, 95%, 96%, 97%, 98%, or 99% identical or higher to SEQ ID NO: 133. In one embodiment, the anti-TIM-3 antibody molecule comprises a heavy chain comprising the amino acid sequence of SEQ ID NO: 113 and a light chain comprising the amino acid sequence of SEQ ID NO: 123. In one embodiment, the anti-TIM-3 antibody molecule comprises a heavy chain comprising the amino acid sequence of SEQ ID NO: 129 and a light chain comprising the amino acid sequence of SEQ ID NO: 133.

In one embodiment, the antibody molecule comprises a heavy chain encoded by the nucleotide sequence of SEQ ID NO: 114, or a nucleotide sequence at least 85%, 90%, 95%, 96%, 97%, 98%, or 99% identical or higher to SEQ ID NO: 114. In one embodiment, the antibody molecule comprises a light chain encoded by the nucleotide sequence of SEQ ID NO: 124, or a nucleotide sequence at least 85%, 90%, 95%, 96%, 97%, 98%, or 99% identical or higher to SEQ ID NO: 124. In one embodiment, the antibody molecule comprises a heavy chain encoded by the nucleotide sequence of SEQ ID NO: 130, or a nucleotide sequence at least 85%, 90%, 95%, 96%, 97%, 98%, or 99% identical or higher to SEQ ID NO: 130. In one embodiment, the antibody molecule comprises a light chain encoded by the nucleotide sequence of SEQ ID NO: 134, or a nucleotide sequence at least 85%, 90%, 95%, 96%, 97%, 98%, or 99% identical or higher to SEQ ID NO: 134. In one embodiment, the antibody molecule comprises a heavy chain encoded by the nucleotide sequence of SEQ ID NO: 114 and a light chain encoded by the nucleotide sequence of SEQ ID NO: 124. In one embodiment, the antibody molecule comprises a heavy chain encoded by the nucleotide sequence of SEQ ID NO: 130 and a light chain encoded by the nucleotide sequence of SEQ ID NO: 134.

The antibody molecules described herein can be made by vectors, host cells, and methods described in US 2015/0218274, incorporated herein by reference in its entirety.

TABLE 5
Amino acid and nucleotide sequences of exemplary anti-TIM-3
antibody molecules

| | | |
|---|---|---|
| ABTIM3-hum11 | | |
| SEQ ID NO: 106 (Kabat) | HCDR1 | SYNMH |
| SEQ ID NO: 107 (Kabat) | HCDR2 | DIYPGNGDTSYNQKFKG |
| SEQ ID NO: 108 (Kabat) | HCDR3 | VGGAFPMDY |
| SEQ ID NO: 109 | HCDR1 | GYTFTSY |
| (Chothia) | | |
| SEQ ID NO: 110 | HCDR2 | YPGNGD |
| (Chothia) | | |
| SEQ ID NO: 108 | HCDR3 | VGGAFPMDY |
| (Chothia) | | |
| SEQ ID NO: 111 | VH | QVQLVQSGAEVKKPGSSVKVSCKASGYTFTSY |
| | | NMHWVRQAPGQGLEWMGDIYPGNGDTSYNQ |
| | | KFKGRVTITADKSTSTVYMELSSLRSEDTAVYY |
| | | CARVGGAFPMDYWGQGTTVTVSS |
| SEQ ID NO: 112 | DNA | CAGGTGCAGCTGGTGCAGTCAGGCGCCGAAG |
| | VH | TGAAGAAACCCGGCTCTAGCGTGAAAGTTTC |
| | | TTGTAAAGCTAGTGGCTACACCTTCACTAGCT |
| | | ATAATATGCACTGGGTTCGCCAGGCCCCAGG |
| | | GCAAGGCCTCGAGTGGATGGGCGATATCTAC |
| | | CCCGGGAACGGCGACACTAGTTATAATCAGA |
| | | AGTTTAAGGGTAGAGTCACTATCACCGCCGA |
| | | TAAGTCTACTAGCACCGTCTATATGGAACTG |
| | | AGTTCCCTGAGGTCTGAGGACACCGCCGTCT |
| | | ACTACTGCGCTAGAGTGGGCGGAGCCTTCCC |
| | | TATGGACTACTGGGGTCAAGGCACTACCGTG |
| | | ACCGTGTCTAGC |
| SEQ ID NO: 113 | Heavy | QVQLVQSGAEVKKPGSSVKVSCKASGYTFTSY |
| | chain | NMHWVRQAPGQGLEWMGDIYPGNGDTSYNQ |
| | | KFKGRVTITADKSTSTVYMELSSLRSEDTAVYY |
| | | CARVGGAFPMDYWGQGTTVTVSSASTKGPSV |
| | | FPLAPCSRSTSESTAALGCLVKDYFPEPVTVSW |
| | | NSGALTSGVHTFPAVLQSSGLYSLSSVVTVPSS |
| | | SLGTKTYTCNVDHKPSNTKVDKRVESKYGPPC |
| | | PPCPAPEFLGGPSVFLFPPKPKDTLMISRTPEVT |
| | | CVVVDVSQEDPEVQFNWYVDGVEVHNAKTKP |
| | | REEQFNSTYRVVSVLTVLHQDWLNGKEYKCK |
| | | VSNKGLPSSIEKTISKAKGQPREPQVYTLPPSQE |
| | | EMTKNQVSLTCLVKGFYPSDIAVEWESNGQPE |
| | | NNYKTTPPVLDSDGSFFLYSRLTVDKSRWQEG |
| | | NVFSCSVMHEALHNHYTQKSLSLSLG |
| SEQ ID NO: 114 | DNA | CAGGTGCAGCTGGTGCAGTCAGGCGCCGAAG |
| | heavy | TGAAGAAACCCGGCTCTAGCGTGAAAGTTTC |
| | chain | TTGTAAAGCTAGTGGCTACACCTTCACTAGCT |
| | | ATAATATGCACTGGGTTCGCCAGGCCCCAGG |
| | | GCAAGGCCTCGAGTGGATGGGCGATATCTAC |
| | | CCCGGGAACGGCGACACTAGTTATAATCAGA |
| | | AGTTTAAGGGTAGAGTCACTATCACCGCCGA |
| | | TAAGTCTACTAGCACCGTCTATATGGAACTG |
| | | AGTTCCCTGAGGTCTGAGGACACCGCCGTCT |
| | | ACTACTGCGCTAGAGTGGGCGGAGCCTTCCC |
| | | TATGGACTACTGGGGTCAAGGCACTACCGTG |
| | | ACCGTGTCTAGCGCTAGCACTAAGGGCCCGT |
| | | CCGTGTTCCCCCTGGCACCTTGTAGCCGGAG |
| | | CACTAGCGAATCCACCGCTGCCCTCGGCTGC |
| | | CTGGTCAAGGATTACTTCCCGGAGCCCGTGA |
| | | CCGTGTCCTGGAACAGCGGAGCCCTGACCTC |
| | | CGGAGTGCACACCTTCCCCGCTGTGCTGCAG |
| | | AGCTCCGGGCTGTACTCGCTGTCGTCGGTGG |
| | | TCACGGTGCCTTCATCTAGCCTGGGTACCAA |
| | | GACCTACACTTGCAACGTGGACCACAAGCCT |
| | | TCCAACACTAAGGTGGACAAGCGCGTCGAAT |
| | | CGAAGTACGGCCCACCGTGCCCGCCTTGTCC |
| | | CGCGCCGGAGTTCCTCGGCGGTCCCTCGGTC |
| | | TTTCTGTTCCCACCGAAGCCCAAGGACACTTT |
| | | GATGATTTCCCGCACCCCTGAAGTGACATGC |
| | | GTGGTCGTGGACGTGTCACAGGAAGATCCGG |
| | | AGGTGCAGTTCAATTGGTACGTGGATGGCGT |
| | | CGAGGTGCACAACGCCAAAACCAAGCCGAG |
| | | GGAGGAGCAGTTCAACTCCACTTACCGCGTC |
| | | GTGTCCGTGCTGACGGTGCTGCATCAGGACT |
| | | GGCTGAACGGGAAGGAGTACAAGTGCAAAG |
| | | TGTCCAACAAGGGACTTCCTAGCTCAATCGA |
| | | AAAGACCATCTCGAAAGCCAAGGGACAGCC |
| | | CCGGGAACCCCAAGTGTATACCCTGCCACCG |
| | | AGCCAGGAAGAAATGACTAAGAACCAAGTC |
| | | TCATTGACTTGCCTTGTGAAGGGCTTCTACCC |
| | | ATCGGATATCGCCGTGGAATGGGAGTCCAAC |
| | | GGCCAGCCGGAAAACAACTACAAGACCACC |
| | | CCTCCGGTGCTGGACTCAGACGGATCCTTCTT |
| | | CCTCTACTCGCGGCTGACCGTGGATAAGAGC |
| | | AGATGGCAGGAGGGAAATGTGTTCAGCTGTT |
| | | CTGTGATGCATGAAGCCCTGCACAACCACTA |
| | | CACTCAGAAGTCCCTGTCCCTCTCCCTGGGA |
| SEQ ID NO: 115 (Kabat) | LCDR1 | RASESVEYYGTSLMQ |
| SEQ ID NO: 116 (Kabat) | LCDR2 | AASNVES |
| SEQ ID NO: 117 (Kabat) | LCDR3 | QQSRKDPST |
| SEQ ID NO: 118 | LCDR1 | SESVEYYGTSL |
| (Chothia) | | |
| SEQ ID NO: 119 | LCDR2 | AAS |
| (Chothia) | | |
| SEQ ID NO: 120 | LCDR3 | SRKDPS |
| (Chothia) | | |
| SEQ ID NO: 121 | VL | AIQLTQSPSSLSASVGDRVTITCRASESVEYYGT |
| | | SLMQWYQQKPGKAPKLLIYAASNVESGVPSRF |
| | | SGSGSGTDFTLTISSLQPEDFATYFCQQSRKDPS |
| | | TFGGGTKVEIK |
| SEQ ID NO: 122 | DNA | GCTATTCAGCTGACTCAGTCACCTAGTAGCCT |
| | VL | GAGCGCTAGTGTGGGCGATAGAGTGACTATC |
| | | ACCTGTAGAGCTAGTGAATCAGTCGAGTACT |
| | | ACGGCACTAGCCTGATGCAGTGGTATCAGCA |
| | | GAAGCCCGGGAAAGCCCCTAAGCTGCTGATC |
| | | TACGCCGCCTCTAACGTGGAATCAGGCGTGC |
| | | CCTCTAGGTTTAGCGGTAGCGGTAGTGGCAC |
| | | CGACTTCACCCTGACTATCTCTAGCCTGCAGC |
| | | CCGAGGACTTCGCTACCTACTTCTGTCAGCA |
| | | GTCTAGGAAGGACCCTAGCACCTTCGGCGGA |
| | | GGCACTAAGGTCGAGATTAAG |
| SEQ ID NO: 123 | Light | AIQLTQSPSSLSASVGDRVTITCRASESVEYYGT |
| | chain | SLMQWYQQKPGKAPKLLIYAASNVESGVPSRF |
| | | SGSGSGTDFTLTISSLQPEDFATYFCQQSRKDPS |
| | | TFGGGTKVEIKRTVAAPSVFIFPPSDEQLKSGTA |
| | | SVVCLLNNFYPREAKVQWKVDNALQSGNSQE |
| | | SVTEQDSKDSTYSLSSTLTLSKADYEKHKVYA |
| | | CEVTHQGLSSPVTKSFNRGEC |
| SEQ ID NO: 124 | DNA | GCTATTCAGCTGACTCAGTCACCTAGTAGCCT |
| | light | GAGCGCTAGTGTGGGCGATAGAGTGACTATC |
| | chain | ACCTGTAGAGCTAGTGAATCAGTCGAGTACT |
| | | ACGGCACTAGCCTGATGCAGTGGTATCAGCA |
| | | GAAGCCCGGGAAAGCCCCTAAGCTGCTGATC |
| | | TACGCCGCCTCTAACGTGGAATCAGGCGTGC |
| | | CCTCTAGGTTTAGCGGTAGCGGTAGTGGCAC |
| | | CGACTTCACCCTGACTATCTCTAGCCTGCAGC |
| | | CCGAGGACTTCGCTACCTACTTCTGTCAGCA |
| | | GTCTAGGAAGGACCCTAGCACCTTCGGCGGA |
| | | GGCACTAAGGTCGAGATTAAGCGTACGGTGG |
| | | CCGCTCCCAGCGTGTTCATCTTCCCCCCCAGC |
| | | GACGAGCAGCTGAAGAGCGGCACCGCCAGC |
| | | GTGGTGTGCCTGCTGAACAACTTCTACCCCC |
| | | GGGAGGCCAAGGTGCAGTGGAAGGTGGACA |
| | | ACGCCCTGCAGAGCGGCAACAGCCAGGAGA |
| | | GCGTCACCGAGCAGGACAGCAAGGACTCCAC |
| | | CTACAGCCTGAGCAGCACCCTGACCCTGAGC |
| | | AAGGCCGACTACGAGAAGCATAAGGTGTAC |
| | | GCCTGCGAGGTGACCCACCAGGGCCTGTCCA |
| | | GCCCCGTGACCAAGAGCTTCAACAGGGGCGA |
| | | GTGC |
| ABTIM3-hum03 | | |
| SEQ ID NO: 106 (Kabat) | HCDR1 | SYNMH |
| SEQ ID NO: 125 (Kabat) | HCDR2 | DIYPGQGDTSYNQKFKG |
| SEQ ID NO: 108 (Kabat) | HCDR3 | VGGAFPMDY |
| SEQ ID NO: 109 | HCDR1 | GYTFTSY |
| (Chothia) | | |
| SEQ ID NO: 126 | HCDR2 | YPGQGD |
| (Chothia) | | |
| SEQ ID NO: 108 | HCDR3 | VGGAFPMDY |
| (Chothia) | | |
| SEQ ID NO: 127 | VH | QVQLVQSGAEVKKPGASVKVSCKASGYTFTSY |
| | | NMHWVRQAPGQGLEWIGDIYPGQGDTSYNQK |
| | | FKGRATMTADKSTSTVYMELSSLRSEDTAVYY |
| | | CARVGGAFPMDYWGQGTLVTVSS |
| SEQ ID NO: 128 | DNA | CAGGTGCAGCTGGTGCAGTCAGGCGCCGAAG |
| | VH | TGAAGAAACCCGGCGCTAGTGTGAAAGTTAG |
| | | CTGTAAAGCTAGTGGCTATACTTTCACTTCTT |
| | | ATAATATGCACTGGGTCCGCCAGGCCCCAGG |
| | | TCAAGGCCTCGAGTGGATCGGCGATATCTAC |
| | | CCCGGTCAAGGCGACACTTCCTATAATCAGA |
| | | AGTTTAAGGGTAGAGCTACTATGACCGCCGA |
| | | TAAGTCTACTICTACCGTCTATATGGAACTGA |
| | | GTTCCCTGAGGTCTGAGGACACCGCCGTCTA |
| | | CTACTGCGCTAGAGTGGGCGGAGCCTTCCCA |
| | | ATGGACTACTGGGGTCAAGGCACCCTGGTCA |
| | | CCGTGTCTAGC |
| SEQ ID NO: 129 | Heavy | QVQLVQSGAEVKKPGASVKVSCKASGYTFTSY |
| | chain | NMHWVRQAPGQGLEWIGDIYPGQGDTSYNQK |
| | | FKGRATMTADKSTSTVYMELSSLRSEDTAVYY |
| | | CARVGGAFPMDYWGQGTLVTVSSASTKGPSV |
| | | FPLAPCSRSTSESTAALGCLVKDYFPEPVTVSW |
| | | NSGALTSGVHTFPAVLQSSGLYSLSSVVTVPSS |
| | | SLGTKTYTCNVDHKPSNTKVDKRVESKYGPPC |
| | | PPCPAPEFLGGPSVFLFPPKPKDTLMISRTPEVT |
| | | CVVVDVSQEDPEVQFNWYVDGVEVHNAKTKP |
| | | REEQFNSTYRVVSVLTVLHQDWLNGKEYKCK |
| | | VSNKGLPSSIEKTISKAKGQPREPQVYTLPPSQE |
| | | EMTKNQVSLTCLVKGFYPSDIAVEWESNGQPE |
| | | NNYKTTPPVLDSDGSFFLYSRLTVDKSRWQEG |
| | | NVFSCSVMHEALHNHYTQKSLSLSLG |
| SEQ ID NO: 130 | DNA | CAGGTGCAGCTGGTGCAGTCAGGCGCCGAAG |
| | heavy | TGAAGAAACCCGGCGCTAGTGTGAAAGTTAG |
| | chain | CTGTAAAGCTAGTGGCTATACTTTCACTTCTT |
| | | ATAATATGCACTGGGTCCGCCAGGCCCCAGG |
| | | TCAAGGCCTCGAGTGGATCGGCGATATCTAC |
| | | CCCGGTCAAGGCGACACTTCCTATAATCAGA |
| | | AGTTTAAGGGTAGAGCTACTATGACCGCCGA |
| | | TAAGTCTACTTCTACCGTCTATATGGAACTGA |
| | | GTTCCCTGAGGTCTGAGGACACCGCCGTCTA |
| | | CTACTGCGCTAGAGTGGGCGGAGCCTTCCCA |
| | | ATGGACTACTGGGGTCAAGGCACCCTGGTCA |
| | | CCGTGTCTAGCGCTAGCACTAAGGGCCCGTC |
| | | CGTGTTCCCCCTGGCACCTTGTAGCCGGAGC |
| | | ACTAGCGAATCCACCGCTGCCCTCGGCTGCC |
| | | TGGTCAAGGATTACTTCCCGGAGCCCGTGAC |
| | | CGTGTCCTGGAACAGCGGAGCCCTGACCTCC |
| | | GGAGTGCACACCTTCCCCGCTGTGCTGCAGA |
| | | GCTCCGGGCTGTACTCGCTGTCGTCGGTGGTC |
| | | ACGGTGCCTTCATCTAGCCTGGGTACCAAGA |
| | | CCTACACTTGCAACGTGGACCACAAGCCTTC |
| | | CAACACTAAGGTGGACAAGCGCGTCGAATCG |
| | | AAGTACGGCCCACCGTGCCCGCCTTGTCCCG |
| | | CGCCGGAGTTCCTCGGCGGTCCCTCGGTCTTT |
| | | CTGTTCCCACCGAAGCCCAAGGACACTTTGA |
| | | TGATTTCCCGCACCCCTGAAGTGACATGCGT |
| | | GGTCGTGGACGTGTCACAGGAAGATCCGGAG |
| | | GTGCAGTTCAATTGGTACGTGGATGGCGTCG |
| | | AGGTGCACAACGCCAAAACCAAGCCGAGGG |
| | | AGGAGCAGTTCAACTCCACTTACCGCGTCGT |
| | | GTCCGTGCTGACGGTGCTGCATCAGGACTGG |
| | | CTGAACGGGAAGGAGTACAAGTGCAAAGTG |
| | | TCCAACAAGGGACTTCCTAGCTCAATCGAAA |
| | | AGACCATCTCGAAAGCCAAGGGACAGCCCCG |
| | | GGAACCCCAAGTGTATACCCTGCCACCGAGC |
| | | CAGGAAGAAATGACTAAGAACCAAGTCTCAT |
| | | TGACTTGCCTTGTGAAGGGCTTCTACCCATCG |
| | | GATATCGCCGTGGAATGGGAGTCCAACGGCC |
| | | AGCCGGAAAACAACTACAAGACCACCCCTCC |
| | | GGTGCTGGACTCAGACGGATCCTTCTTCCTCT |
| | | ACTCGCGGCTGACCGTGGATAAGAGCAGATG |
| | | GCAGGAGGGAAATGTGTTCAGCTGTTCTGTG |
| | | ATGCATGAAGCCCTGCACAACCACTACACTC |
| | | AGAAGTCCCTGTCCCTCTCCCTGGGA |
| SEQ ID NO: 115 (Kabat) | LCDR1 | RASESVEYYGTSLMQ |
| SEQ ID NO: 116 (Kabat) | LCDR2 | AASNVES |
| SEQ ID NO: 117 (Kabat) | LCDR3 | QQSRKDPST |
| SEQ ID NO: 118 | LCDR1 | SESVEYYGTSL |
| (Chothia) | | |
| SEQ ID NO: 119 | LCDR2 | AAS |
| (Chothia) | | |
| SEQ ID NO: 120 | LCDR3 | SRKDPS |
| (Chothia) | | |
| SEQ ID NO: 131 | VL | DIVLTQSPDSLAVSLGERATINCRASESVEYYG |
| | | TSLMQWYQQKPGQPPKLLIYAASNVESGVPDR |
| | | FSGSGSGTDFTLTISSLQAEDVAVYYCQQSRKD |
| | | PSTFGGGTKVEIK |
| SEQ ID NO: 132 | DNA | GATATCGTCCTGACTCAGTCACCCGATAGCC |
| | VL | TGGCCGTCAGCCTGGGCGAGCGGGCTACTAT |
| | | TAACTGTAGAGCTAGTGAATCAGTCGAGTAC |
| | | TACGGCACTAGCCTGATGCAGTGGTATCAGC |
| | | AGAAGCCCGGTCAACCCCCTAAGCTGCTGAT |
| | | CTACGCCGCCTCTAACGTGGAATCAGGCGTG |
| | | CCCGATAGGTTTAGCGGTAGCGGTAGTGGCA |
| | | CCGACTTCACCCTGACTATTAGTAGCCTGCA |
| | | GGCCGAGGACGTGGCCGTCTACTACTGTCAG |
| | | CAGTCTAGGAAGGACCCTAGCACCTTCGGCG |
| | | GAGGCACTAAGGTCGAGATTAAG |
| SEQ ID NO: 133 | Light | DIVLTQSPDSLAVSLGERATINCRASESVEYYG |
| | chain | TSLMQWYQQKPGQPPKLLIYAASNVESGVPDR |
| | | FSGSGSGTDFTLTISSLQAEDVAVYYCQQSRKD |
| | | PSTFGGGTKVEIKRTVAAPSVFIFPPSDEQLKSG |
| | | TASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 134 | DNA | GATATCGTCCTGACTCAGTCACCCGATAGCC |
| | light | TGGCCGTCAGCCTGGGCGAGCGGGCTACTAT |
| | chain | TAACTGTAGAGCTAGTGAATCAGTCGAGTAC |
| | | TACGGCACTAGCCTGATGCAGTGGTATCAGC |
| | | AGAAGCCCGGTCAACCCCCTAAGCTGCTGAT |
| | | CTACGCCGCCTCTAACGTGGAATCAGGCGTG |
| | | CCCGATAGGTTTAGCGGTAGCGGTAGTGGCA |
| | | CCGACTTCACCCTGACTATTAGTAGCCTGCA |
| | | GGCCGAGGACGTGGCCGTCTACTACTGTCAG |
| | | CAGTCTAGGAAGGACCCTAGCACCTTCGGCG |
| | | GAGGCACTAAGGTCGAGATTAAGCGTACGGT |
| | | GGCCGCTCCCAGCGTGTTCATCTTCCCCCCCA |
| | | GCGACGAGCAGCTGAAGAGCGGCACCGCCA |
| | | GCGTGGTGTGCCTGCTGAACAACTTCTACCC |
| | | CCGGGAGGCCAAGGTGCAGTGGAAGGTGGA |
| | | CAACGCCCTGCAGAGCGGCAACAGCCAGGA |
| | | GAGCGTCACCGAGCAGGACAGCAAGGACTC |
| | | CACCTACAGCCTGAGCAGCACCCTGACCCTG |
| | | AGCAAGGCCGACTACGAGAAGCATAAGGTG |
| | | TACGCCTGCGAGGTGACCCACCAGGGCCTGT |
| | | CCAGCCCCGTGACCAAGAGCTTCAACAGGGG |
| | | CGAGTGC |

In some embodiments, the TIM-3 inhibitor is administered at a dose of about 50 mg to about 100 mg, about 200 mg to about 250 mg, about 500 mg to about 1000 mg, or about 1000 mg to about 1500 mg. In embodiments, the TIM-3 inhibitor is administered once every 4 weeks. In other embodiments, the TIM-3 inhibitor is administered at a dose of about 50 mg to about 100 mg once every four weeks. In other embodiments, the TIM-3 inhibitor is administered at a dose of about 200 mg to about 250 mg once every four weeks. In other embodiments, the TIM-3 inhibitor is administered at a dose of about 500 mg to about 1000 mg once every four weeks. In other embodiments, the TIM-3 inhibitor is administered at a dose of about 1000 mg to about 1500 mg once every four weeks.

### Other Exemplary TIM-3 Inhibitors

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table 6. APE5137, APE5121, and other anti-TIM-3 antibodies are disclosed in WO 2016/161270, incorporated herein by reference in its entirety.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain or light chain variable region sequence, or the heavy chain or light chain sequence of F38-2E2.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated herein by reference in their entirety.

In one embodiment, the anti-TIM-3 antibody is an antibody that competes for binding with, and/or binds to the same epitope on TIM-3 as, one of the anti-TIM-3 antibodies described herein.

TABLE 6
Amino acid sequences of other exemplary anti-TIM-3
antibody molecules

| | | |
|---|---|---|
| APE5137 | | |
| SEQ ID NO: | VH | EVQLLESGGGLVQPGGSLRLSCAAASGFTFSSYDMSWVR |
| 135 | | QAPGKGLDWVSTISGGGTYTYYQDSVKGRFTISRDNSKN |
| | | TLYLQMNSLRAEDTAVYYCASMDYWGQGTTVTVSSA |
| SEQ ID NO: | VL | DIQMTQSPSSLSASVGDRVTITCRASQSIRRYLNWYHQKP |
| 136 | | GKAPKLLIYGASTLQSGVPSRFSGSGSGTDFTLTISSLQPE |
| | | DFAVYYCQQSHSAPLTFGGGTKVEIKR |
| APE5121 | | |
| SEQ ID NO: | VH | EVQVLESGGGLVQPGGSLRLYCVASGFTFSGSYAMSWV |
| 137 | | RQAPGKGLEWVSAISGSGGSTYYADSVKGRFTISRDNSK |
| | | NTLYLQMNSLRAEDTAVYYCAKKYYVGPADYWGQGTL |
| | | VTVSSG |
| SEQ ID NO: | VL | DIVMTQSPDSLAVSLGERATINCKSSQSVLYSSNNKNYLA |
| 138 | | WYQHKPGQPPKLLIYWASTRESGVPDRFSGSGSGTDFTL |
| | | TISSLQAEDVAVYYCQQYYSSPLTFGGGTKIEVK |

### GITR Agonists

Glucocorticoid-induced TNFR-related protein (GITR) is a member of the Tumor Necrosis Factor Superfamily (TNFRSF). GITR expression is detected constitutively on murine and human CD4+CD25+ regulatory T cells which can be further increased upon activation. In contrast, effector CD4+CD25− T cells and CD8+CD25− T cells express low to undetectable levels of GITR, which is rapidly upregulated following T cell receptor activation. Expression of GITR has also been detected on activated NK cells, dendritic cells, and macrophages. Signal transduction pathway downstream of GITR has been shown to involve MAPK and the canonical NFκB pathways. Various TRAF family members have been implicated as signaling intermediates downstream of GITR (Nocentini et al. (2005) Eur. J. Immunol. 35:1016-1022).

Cellular activation through GITR is believed to serve several functions depending on the cell type and microenvironment including, but not limited to, costimulation to augment proliferation and effector function, inhibition of suppression by regulatory T cells, and protection from activation-induced cell death (Shevach and Stephens (2006) Nat. Rev. Immunol. 6:613-618). An agonistic monoclonal antibody against mouse GITR effectively induced tumor-specific immunity and eradicated established tumors in a mouse syngeneic tumor model (Ko et al. (2005) J. Exp. Med. 202:885-891).

In certain embodiments, the combination or method according to the invention described herein comprises a GITR agonist as I-O therapeutic agent. In some embodiments, the GITR agonist is chosen from GWN323 (NVS), BMS-986156, MK-4166 or MK-1248 (Merck), TRX518 (Leap Therapeutics), INCAGN1876 (Incyte/Agenus), AMG 228 (Amgen) or INBRX-110 (Inhibrx).

### Exemplary GITR Agonists

In one embodiment, the GITR agonist is an anti-GITR antibody molecule. In one embodiment, the GITR agonist is an anti-GITR antibody molecule as described in WO 2016/057846, published on Apr. 14, 2016, entitled “Compositions and Methods of Use for Augmented Immune Response and Cancer Therapy,” incorporated herein by reference in its entirety.

In one embodiment, the anti-GITR antibody molecule comprises at least one, two, three, four, five or six complementarity determining regions (CDRs) (or collectively all of the CDRs) from a heavy and light chain variable region comprising an amino acid sequence shown in Table 7 (e.g., from the heavy and light chain variable region sequences of MAB7 disclosed in Table 7), or encoded by a nucleotide sequence shown in Table 7. In some embodiments, the CDRs are according to the Kabat definition (e.g., as set out in Table 7). In some embodiments, the CDRs are according to the Chothia definition (e.g., as set out in Table 7). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 7, or encoded by a nucleotide sequence shown in Table 7.

In one embodiment, the anti-GITR antibody molecule comprises a heavy chain variable region (VH) comprising a VHCDR1 amino acid sequence of SEQ ID NO: 147, a VHCDR2 amino acid sequence of SEQ ID NO: 149, and a VHCDR3 amino acid sequence of SEQ ID NO: 151; and a light chain variable region (VL) comprising a VLCDR1 amino acid sequence of SEQ ID NO: 152, a VLCDR2 amino acid sequence of SEQ ID NO: 154, and a VLCDR3 amino acid sequence of SEQ ID NO: 156, each disclosed in Table 7.

In one embodiment, the anti-GITR antibody molecule comprises a VH comprising the amino acid sequence of SEQ ID NO: 139, or an amino acid sequence at least 85%, 90%, 95%, 96%, 97%, 98%, or 99% identical or higher to SEQ ID NO: 139. In one embodiment, the anti-GITR antibody molecule comprises a VL comprising the amino acid sequence of SEQ ID NO: 140, or an amino acid sequence at least 85%, 90%, 95%, 96%, 97%, 98%, or 99% identical or higher to SEQ ID NO: 140. In one embodiment, the anti-GITR antibody molecule comprises a VH comprising the amino acid sequence of SEQ ID NO: 139 and a VL comprising the amino acid sequence of SEQ ID NO: 140.

In one embodiment, the antibody molecule comprises a VH encoded by the nucleotide sequence of SEQ ID NO: 143, or a nucleotide sequence at least 85%, 90%, 95%, 96%, 97%, 98%, or 99% identical or higher to SEQ ID NO: 143. In one embodiment, the antibody molecule comprises a VL encoded by the nucleotide sequence of SEQ ID NO: 144, or a nucleotide sequence at least 85%, 90%, 95%, 96%, 97%, 98%, or 99% identical or higher to SEQ ID NO: 144. In one embodiment, the antibody molecule comprises a VH encoded by the nucleotide sequence of SEQ ID NO: 143 and a VL encoded by the nucleotide sequence of SEQ ID NO: 144.

In one embodiment, the anti-GITR antibody molecule comprises a heavy chain comprising the amino acid sequence of SEQ ID NO: 141, or an amino acid sequence at least 85%, 90%, 95%, 96%, 97%, 98%, or 99% identical or higher to SEQ ID NO: 141. In one embodiment, the anti-GITR antibody molecule comprises a light chain comprising the amino acid sequence of SEQ ID NO: 142, or an amino acid sequence at least 85%, 90%, 95%, 96%, 97%, 98%, or 99% identical or higher to SEQ ID NO: 142. In one embodiment, the anti-GITR antibody molecule comprises a heavy chain comprising the amino acid sequence of SEQ ID NO: 141 and a light chain comprising the amino acid sequence of SEQ ID NO: 142.

In one embodiment, the antibody molecule comprises a heavy chain encoded by the nucleotide sequence of SEQ ID NO: 145, or a nucleotide sequence at least 85%, 90%, 95%, 96%, 97%, 98%, or 99% identical or higher to SEQ ID NO: 145. In one embodiment, the antibody molecule comprises a light chain encoded by the nucleotide sequence of SEQ ID NO: 146, or a nucleotide sequence at least 85%, 90%, 95%, 96%, 97%, 98%, or 99% identical or higher to SEQ ID NO: 146. In one embodiment, the antibody molecule comprises a heavy chain encoded by the nucleotide sequence of SEQ ID NO: 145 and a light chain encoded by the nucleotide sequence of SEQ ID NO: 146.

The antibody molecules described herein can be made by vectors, host cells, and methods described in WO 2016/057846, incorporated herein by reference in its entirety.

TABLE 7
Amino acid and nucleotide sequences of exemplary anti-GITR
antibody molecule

| | | |
|---|---|---|
| MAB7 | | |
| SEQ ID NO: 139 | VH | EVOLVESGGGLVQSGGSLRLSCAASGFSLSSYG |
| | | VDWVRQAPGKGLEWVGVIWGGGGTYYASSLM |
| | | GRFTISRDNSKNTLYLQMNSLRAEDTAVYYCAR |
| | | HAYGHDGGFAMDYWGQGTLVTVSS |
| SEQ ID NO: 140 | VL | EIVMTQSPATLSVSPGERATLSCRASESVSSNVA |
| | | WYQQRPGQAPRLLIYGASNRATGIPARFSGSGS |
| | | GTDFTLTISRLEPEDFAVYYCGQSYSYPFTFGQG |
| | | TKLEIK |
| SEQ ID NO: 141 | Heavy | EVOLVESGGGLVQSGGSLRLSCAASGFSLSSYG |
| | Chain | VDWVRQAPGKGLEWVGVIWGGGGTYYASSLM |
| | | GRFTISRDNSKNTLYLQMNSLRAEDTAVYYCAR |
| | | HAYGHDGGFAMDYWGQGTLVTVSSASTKGPS |
| | | VFPLAPSSKSTSGGTAALGCLVKDYFPEPVTVS |
| | | WNSGALTSGVHTFPAVLQSSGLYSLSSVVTVPS |
| | | SSLGTQTYICNVNHKPSNTKVDKRVEPKSCDKT |
| | | HTCPPCPAPELLGGPSVFLFPPKPKDTLMISRTPE |
| | | VTCVVVDVSHEDPEVKFNWYVDGVEVHNAKT |
| | | KPREEQYNSTYRVVSVLTVLHQDWLNGKEYKC |
| | | KVSNKALPAPIEKTISKAKGQPREPQVYTLPPSR |
| | | EEMTKNQVSLTCLVKGFYPSDIAVEWESNGQPE |
| | | NNYKTTPPVLDSDGSFFLYSKLTVDKSRWQQG |
| | | NVFSCSVMHEALHNHYTQKSLSLSPGK |
| SEQ ID NO: 142 | Light | EIVMTQSPATLSVSPGERATLSCRASESVSSNVA |
| | Chain | WYQQRPGQAPRLLIYGASNRATGIPARFSGSGS |
| | | GTDFTLTISRLEPEDFAVYYCGQSYSYPFTFGQG |
| | | TKLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGEC |
| SEQ ID NO: 143 | DNA | GAGGTGCAGCTGGTGGAATCTGGCGGCGGAC |
| | VH | TGGTGCAGTCCGGCGGCTCTCTGAGACTGTCT |
| | | TGCGCTGCCTCCGGCTTCTCCCTGTCCTCTTAC |
| | | GGCGTGGACTGGGTGCGACAGGCCCCTGGCA |
| | | AGGGCCTGGAATGGGTGGGAGTGATCTGGGG |
| | | CGGAGGCGGCACCTACTACGCCTCTTCCCTGA |
| | | TGGGCCGGTTCACCATCTCCCGGGACAACTCC |
| | | AAGAACACCCTGTACCTGCAGATGAACTCCCT |
| | | GCGGGCCGAGGACACCGCCGTGTACTACTGC |
| | | GCCAGACACGCCTACGGCCACGACGGCGGCT |
| | | TCGCCATGGATTATTGGGGCCAGGGCACCCTG |
| | | GTGACAGTGTCCTCC |
| SEQ ID NO: 144 | DNA | GAGATCGTGATGACCCAGTCCCCCGCCACCCT |
| | VL | GTCTGTGTCTCCCGGCGAGAGAGCCACCCTGA |
| | | GCTGCAGAGCCTCCGAGTCCGTGTCCTCCAAC |
| | | GTGGCCTGGTATCAGCAGAGACCTGGTCAGG |
| | | CCCCTCGGCTGCTGATCTACGGCGCCTCTAAC |
| | | CGGGCCACCGGCATCCCTGCCAGATTCTCCGG |
| | | CTCCGGCAGCGGCACCGACTTCACCCTGACCA |
| | | TCTCCCGGCTGGAACCCGAGGACTTCGCCGTG |
| | | TACTACTGCGGCCAGTCCTACTCATACCCCTT |
| | | CACCTTCGGCCAGGGCACCAAGCTGGAAATC |
| | | AAG |
| SEQ ID | DNA | GAGGTGCAGCTGGTGGAATCTGGCGGCGGACTGGTG |
| NO: 145 | Heavy | CAGTCCGGCGGCTCTCTGAGACTGTCTTGCGCTGCCT |
| | Chain | CCGGCTTCTCCCTGTCCTCTTACGGCGTGGACTGGGT |
| | | GCGACAGGCCCCTGGCAAGGGCCTGGAATGGGTGGG |
| | | AGTGATCTGGGGCGGAGGCGGCACCTACTACGCCTC |
| | | TTCCCTGATGGGCCGGTTCACCATCTCCCGGGACAAC |
| | | TCCAAGAACACCCTGTACCTGCAGATGAACTCCCTG |
| | | CGGGCCGAGGACACCGCCGTGTACTACTGCGCCAGA |
| | | CACGCCTACGGCCACGACGGCGGCTTCGCCATGGAT |
| | | TATTGGGGCCAGGGCACCCTGGTGACAGTGTCCTCC |
| | | GCTAGCACCAAGGGCCCAAGTGTGTTTCCCCTGGCC |
| | | CCCAGCAGCAAGTCTACTTCCGGCGGAACTGCTGCC |
| | | CTGGGTTGCCTGGTGAAGGACTACTTCCCCGAGCCC |
| | | GTGACAGTGTCCTGGAACTCTGGGGCTCTGACTTCCG |
| | | GCGTGCACACCTTCCCCGCCGTGCTGCAGAGCAGCG |
| | | GCCTGTACAGCCTGAGCAGCGTGGTGACAGTGCCCT |
| | | CCAGCTCTCTGGGAACCCAGACCTATATCTGCAACG |
| | | TGAACCACAAGCCCAGCAACACCAAGGTGGACAAG |
| | | AGAGTGGAGCCCAAGAGCTGCGACAAGACCCACAC |
| | | CTGCCCCCCCTGCCCAGCTCCAGAACTGCTGGGAGG |
| | | GCCTTCCGTGTTCCTGTTCCCCCCCAAGCCCAAGGAC |
| | | ACCCTGATGATCAGCAGGACCCCCGAGGTGACCTGC |
| | | GTGGTGGTGGACGTGTCCCACGAGGACCCAGAGGTG |
| | | AAGTTCAACTGGTACGTGGACGGCGTGGAGGTGCAC |
| | | AACGCCAAGACCAAGCCCAGAGAGGAGCAGTACAA |
| | | CAGCACCTACAGGGTGGTGTCCGTGCTGACCGTGCT |
| | | GCACCAGGACTGGCTGAACGGCAAAGAATACAAGT |
| | | GCAAAGTCTCCAACAAGGCCCTGCCAGCCCCAATCG |
| | | AAAAGACAATCAGCAAGGCCAAGGGCCAGCCACGG |
| | | GAGCCCCAGGTGTACACCCTGCCCCCCAGCCGGGAG |
| | | GAGATGACCAAGAACCAGGTGTCCCTGACCTGTCTG |
| | | GTGAAGGGCTTCTACCCCAGCGATATCGCCGTGGAG |
| | | TGGGAGAGCAACGGCC |
| SEQ ID NO: 146 | DNA | GAGATCGTGATGACCCAGTCCCCCGCCACCCT |
| | Light | GTCTGTGTCTCCCGGCGAGAGAGCCACCCTGA |
| | Chain | GCTGCAGAGCCTCCGAGTCCGTGTCCTCCAAC |
| | | GTGGCCTGGTATCAGCAGAGACCTGGTCAGG |
| | | CCCCTCGGCTGCTGATCTACGGCGCCTCTAAC |
| | | CGGGCCACCGGCATCCCTGCCAGATTCTCCGG |
| | | CTCCGGCAGCGGCACCGACTTCACCCTGACCA |
| | | TCTCCCGGCTGGAACCCGAGGACTTCGCCGTG |
| | | TACTACTGCGGCCAGTCCTACTCATACCCCTT |
| | | CACCTTCGGCCAGGGCACCAAGCTGGAAATC |
| | | AAGCGTACGGTGGCCGCTCCCAGCGTGTTCAT |
| | | CTTCCCCCCCAGCGACGAGCAGCTGAAGAGC |
| | | GGCACCGCCAGCGTGGTGTGCCTGCTGAACA |
| | | ACTTCTACCCCCGGGAGGCCAAGGTGCAGTG |
| | | GAAGGTGGACAACGCCCTGCAGAGCGGCAAC |
| | | AGCCAGGAGAGCGTCACCGAGCAGGACAGCA |
| | | AGGACTCCACCTACAGCCTGAGCAGCACCCTG |
| | | ACCCTGAGCAAGGCCGACTACGAGAAGCATA |
| | | AGGTGTACGCCTGCGAGGTGACCCACCAGGG |
| | | CCTGTCCAGCCCCGTGACCAAGAGCTTCAACA |
| | | GGGGCGAGTGC |
| SEQ ID NO: 147 | HCDR | SYGVD |
| (KABAT) | 1 | |
| SEQ ID NO: 148 | HCDR | GFSLSSY |
| (CHOTHIA) | 1 | |
| SEQ ID NO: 149 | HCDR | VIWGGGGTYYASSLMG |
| (KABAT) | 2 | |
| SEQ ID NO: 150 | HCDR | WGGGG |
| (CHOTHIA) | 2 | |
| SEQ ID NO: 151 | HCDR | HAYGHDGGFAMDY |
| (KABAT) | 3 | |
| SEQ ID NO: 151 | HCDR | HAYGHDGGFAMDY |
| (CHOTHIA) | 3 | |
| SEQ ID NO: 152 | LCDR1 | RASESVSSNVA |
| (KABAT) | | |
| SEQ ID NO: 153 | LCDR1 | SESVSSN |
| (CHOTHIA) | | |
| SEQ ID NO: 154 | LCDR2 | GASNRAT |
| (KABAT) | | |
| SEQ ID NO: 155 | LCDR2 | GAS |
| (CHOTHIA) | | |
| SEQ ID NO: 156 | LCDR3 | GQSYSYPFT |
| (KABAT) | | |
| SEQ ID NO: 157 | LCDR3 | SYSYPF |
| (CHOTHIA) | | |

In some embodiments, the GITR agonist is administered at a dose of about 2 mg to about 600 mg (e.g., about 5 mg to about 500 mg). In some embodiments, the GITR agonist is administered once every week. In other embodiments, the GITR agonist is administered once every three weeks. In other embodiments, the GITR agonist is administered once every six weeks.

In some embodiments, the GITR agonist is administered at a dose of about 2 mg to about 10 mg (e.g., about 5 mg), about 5 mg to about 20 mg (e.g., about 10 mg), about 20 mg to about 40 mg (e.g., about 30 mg), about 50 mg to about 100 mg (e.g., about 60 mg), about 100 mg to about 200 mg (e.g., about 150 mg), about 200 mg to about 400 mg (e.g., about 300 mg), or about 400 mg to about 600 mg (e.g., about 500 mg), once every week.

In some embodiments, the GITR agonist is administered at a dose of about 2 mg to about 10 mg (e.g., about 5 mg), about 5 mg to about 20 mg (e.g., about 10 mg), about 20 mg to about 40 mg (e.g., about 30 mg), about 50 mg to about 100 mg (e.g., about 60 mg), about 100 mg to about 200 mg (e.g., about 150 mg), about 200 mg to about 400 mg (e.g., about 300 mg), or about 400 mg to about 600 mg (e.g., about 500 mg), once every three weeks.

In some embodiments, the GITR agonist is administered at a dose of about 2 mg to about 10 mg (e.g., about 5 mg), about 5 mg to about 20 mg (e.g., about 10 mg), about 20 mg to about 40 mg (e.g., about 30 mg), about 50 mg to about 100 mg (e.g., about 60 mg), about 100 mg to about 200 mg (e.g., about 150 mg), about 200 mg to about 400 mg (e.g., about 300 mg), or about 400 mg to about 600 mg (e.g., about 500 mg), once every six weeks.

In some embodiments, three doses of the GITR agonist are administered over a period of three weeks followed by a nine-week pause. In some embodiments, four doses of the GITR agonist are administered over a period of twelve weeks followed by a nine-week pause. In some embodiments, four doses of the GITR agonists are administered over a period of twenty-one or twenty-four weeks followed by a nine-week pause.

### Other Exemplary GITR Agonists

In one embodiment, the anti-GITR antibody molecule is BMS-986156 (Bristol-Myers Squibb), also known as BMS 986156 or BMS986156. BMS-986156 and other anti-GITR antibodies are disclosed, e.g., in U.S. Pat. No. 9,228,016 and WO 2016/196792, incorporated herein by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of BMS-986156, e.g., as disclosed in Table 8.

In one embodiment, the anti-GITR antibody molecule is MK-4166 or MK-1248 (Merck). MK-4166, MK-1248, and other anti-GITR antibodies are disclosed, e.g., in U.S. Pat. No. 8,709,424, WO 2011/028683, WO 2015/026684, and Mahne et al. Cancer Res. 2017; 77(5):1108-1118, incorporated herein by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of MK-4166 or MK-1248.

In one embodiment, the anti-GITR antibody molecule is TRX518 (Leap Therapeutics). TRX518 and other anti-GITR antibodies are disclosed, e.g., in U.S. Pat. Nos. 7,812,135, 8,388,967, 9,028,823, WO 2006/105021, and Ponte J et al. (2010) Clinical Immunology; 135:S96, incorporated herein by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of TRX518.

In one embodiment, the anti-GITR antibody molecule is INCAGN1876 (Incyte/Agenus). INCAGN1876 and other anti-GITR antibodies are disclosed, e.g., in US 2015/0368349 and WO 2015/184099, incorporated herein by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of INCAGN1876.

In one embodiment, the anti-GITR antibody molecule is AMG 228 (Amgen). AMG 228 and other anti-GITR antibodies are disclosed, e.g., in U.S. Pat. No. 9,464,139 and WO 2015/031667, incorporated herein by reference in their entirety. In one embodiment, the anti-GITR antibody molecule comprises one or more of the CDR sequences (or collectively all of the CDR sequences), the heavy chain or light chain variable region sequence, or the heavy chain or light chain sequence of AMG 228.

In one embodiment, the anti-GITR antibody molecule is INBRX-110 (Inhibrx). INBRX-110 and other anti-GITR antibodies are disclosed, e.g., in US 2017/0022284 and WO 2017/015623, incorporated herein by reference in their entirety. In one embodiment, the GITR agonist comprises one or more of the CDR sequences (or collectively all of the CDR sequences), the heavy chain or light chain variable region sequence, or the heavy chain or light chain sequence of INBRX-110.

In one embodiment, the GITR agonist (e.g., a fusion protein) is MEDI 1873 (MedImmune), also known as MEDI1873. MEDI 1873 and other GITR agonists are disclosed, e.g., in US 2017/0073386, WO 2017/025610, and Ross et al. Cancer Res 2016; 76(14 Suppl): Abstract nr 561, incorporated herein by reference in their entirety. In one embodiment, the GITR agonist comprises one or more of an IgG Fc domain, a functional multimerization domain, and a receptor binding domain of a glucocorticoid-induced TNF receptor ligand (GITRL) of MEDI 1873.

In one embodiment, the anti-GITR antibody molecule is an anti-GITR antibody molecule disclosed in WO 2013/039954, incorporated herein by reference in its entirety. In an embodiment, the anti-GITR antibody molecule is an anti-GITR antibody molecule disclosed in US 2014/0072566, incorporated herein by reference in its entirety.

Further known GITR agonists (e.g., anti-GITR antibodies) include those described, e.g., in WO 2016/054638, incorporated herein by reference in its entirety.

In one embodiment, the anti-GITR antibody is an antibody that competes for binding with, and/or binds to the same epitope on GITR as, one of the anti-GITR antibodies described herein.

In one embodiment, the GITR agonist is a peptide that activates the GITR signaling pathway. In one embodiment, the GITR agonist is an immunoadhesin binding fragment (e.g., an immunoadhesin binding fragment comprising an extracellular or GITR binding portion of GITRL) fused to a constant region (e.g., an Fc region of an immunoglobulin sequence).

TABLE 8
Amino acid sequence of other exemplary anti-GITR
antibody molecules

| | | |
|---|---|---|
| BMS-986156 | | |
| SEQ ID NO: 158 | VH | QVQLVESGGGVVQPGRSLRLSCAASGFTFSSYGMH |
| | | WVRQAPGKGLEWVAVIWYEGSNKYYADSVKGRFT |
| | | ISRDNSKNTLYLQMNSLRAEDTAVYYCARGGSMVR |
| | | GDYYYGMDVWGQGTTVTVSS |
| SEQ ID NO: 159 | VL | AIQLTQSPSSLSASVGDRVTITCRASQGISSALAWYQ |
| | | QKPGKAPKLLIYDASSLESGVPSRFSGSGSGTDFTLTI |
| | | SSLQPEDFATYYCQQFNSYPYTFGQGTKLEIK |

### TGF-β Inhibitors

In certain embodiments, the combination or method described herein comprises a PSMA therapeutic agent as described above and a transforming growth factor beta (also known as TGF-β TGFβ, TGFb, or TGF-beta, used interchangeably herein) inhibitor as I-O therapeutic agent.

TGF-β belongs to a large family of structurally-related cytokines including, e.g., bone morphogenetic proteins (BMPs), growth and differentiation factors, activins and inhibins. In some embodiments, the TGF-β inhibitors described herein can bind and/or inhibit one or more isoforms of TGF-β (e.g., one, two, or all of TGF-β1, TGF-β2, or TGF-β3).

Under normal conditions, TGF-β maintains homeostasis and limits the growth of epithelial, endothelial, neuronal and hematopoietic cell lineages, e.g., through the induction of anti-proliferative and apoptotic responses. Canonical and non-canonical signaling pathways are involved in cellular responses to TGF-β. Activation of the TGF-β/Smad canonical pathway can mediate the anti-proliferative effects of TGF-β. The non-canonical TGF-β pathway can activate additional intra-cellular pathways, e.g., mitogen-activated protein kinases (MAPK), phosphatidylinositol 3 kinase/Protein Kinase B, Rho-like GTPases (Tian et al. Cell Signal. 2011; 23(6):951-62; Blobe et al. N Engl J Med. 2000; 342(18):1350-8), thus modulating epithelial to mesenchymal transition (EMT) and/or cell motility.

Alterations of the TGF-β signaling pathway are associated with human diseases, e.g., cancers, cardio-vascular diseases, fibrosis, reproductive disorders, and wound healing. Without wishing to be bound by theory, it is believed that in some embodiments, the role of TGF-β in cancer is dependent on the disease setting (e.g., tumor stage and genetic alteration) and/or cellular context. For example, in late stages of cancer, TGF-β can modulate a cancer-related process, e.g., by promoting tumor growth (e.g., inducing EMT), blocking anti-tumor immune responses, increasing tumor-associated fibrosis, or enhancing angiogenesis (Wakefield and Hill Nat Rev Cancer. 2013; 13(5):328-41). In certain embodiments, a combination or method comprising a TGF-β inhibitor as O—I therapeutic agent described herein is used to treat a cancer in a late stage, a metastatic cancer, or an advanced cancer.

Preclinical evidence indicates that TGF-β plays an important role in immune regulation (Wojtowicz-Praga Invest New Drugs. 2003; 21(1):21-32; Yang et al. Trends Immunol. 2010; 31(6):220-7). TGF-β can down-regulate the host-immune response via several mechanisms, e.g., shift of the T-helper balance toward Th2 immune phenotype; inhibition of anti-tumoral Th1 type response and M1-type macrophages; suppression of cytotoxic CD8+ T lymphocytes (CTL), NK lymphocytes and dendritic cell functions, generation of CD4+CD25+ T-regulatory cells; or promotion of M2-type macrophages with pro-tumoral activity mediated by secretion of immunosuppressive cytokines (e.g., IL10 or VEGF), pro-inflammatory cytokines (e.g., IL6, TNFα, or IL1) and generation of reactive oxygen species (ROS) with genotoxic activity (Yang et al. Trends Immunol. 2010; 31(6):220-7; Truty and Urrutia Pancreatology. 2007; 7(5-6):423-35; Achyut et al Gastroenterology. 2011; 141(4):1167-78).

In some embodiments, the TGF-β inhibitor is used in combination with the PSMA therapeutic agent, such as radiolabeled Compound I, and, in addition, a PD-1 inhibitor, and one or more (e.g., two, three, four, or all) of LAG-3 inhibitor, a GITR agonist, a c-MET inhibitor, an IDO inhibitor, or an A2aR antagonist. In some embodiments, the combination or method may be used to treat a pancreatic cancer, a colorectal cancer, a gastric cancer, a prostate cancer, or a melanoma (e.g., a refractory melanoma).

In some preferred embodiments, the combination or method according to the invention is used to treat PSMA expressing cancer.

In some embodiments, the TGF-β inhibitor is chosen from fresolimumab or XOMA 089.

### Exemplary TGF-β Inhibitors

In some embodiments, the TGF-β inhibitor comprises XOMA 089, or a compound disclosed in International Application Publication No. WO 2012/167143, incorporated herein by reference in its entirety.

XOMA 089 is also known as XPA.42.089. XOMA 089 is a fully human monoclonal antibody that specifically binds and neutralizes TGF-beta 1 and 2 ligands.

The heavy chain variable region of XOMA 089 has
the amino acid sequence of:
(SEQ ID NO: 162)
QVQLVQSGAEVKKPGSSVKVSCKASGGTFSSYAISWVRQAPGQGLEWMGGI
IPIFGTANYAQKFQGRVTITADESTSTAYMELSSLRSEDTAVYYCARGLWE
VRALPSVYWGQGTLVTVSS
(disclosed as SEQ ID NO: 6 in WO 2012/167143).
The light chain variable region of XOMA 089 has
the amino acid sequence of:
(SEQ ID NO: 163)
SYELTQPPSVSVAPGQTARITCGANDIGSKSVHWYQQKAGQAPVLVVSEDI
IRPSGIPERISGSNSGNTATLTISRVEAGDEADYYCQVWDRDSDQYVFGTG
TKVTVLG
(disclosed as SEQ ID NO: 8 in WO 2012/167143).

XOMA 089 binds with high affinity to the human TGF-β isoforms. Generally, XOMA 089 binds with high affinity to TGF-β1 and TGF-β2, and to a lesser extent to TGF-β3. In Biacore assays, the K_{D} of XOMA 089 on human TGF-β is 14.6 pM for TGF-β1, 67.3 pM for TGF-β2, and 948 pM for TGF-β3. Given the high affinity binding to all three TGF-β isoforms, in certain embodiments, XOMA 089 is expected to bind to TGF-β1, 2 and 3 at a dose of XOMA 089 as described herein. XOMA 089 cross-reacts with rodent and cynomolgus monkey TGF-β and shows functional activity in vitro and in vivo, making rodent and cynomolgus monkey relevant species for toxicology studies.

Without wishing to be bound by theory, it is believed that in some embodiments, resistance to PD-1 immunotherapy is associated with the presence of a transcriptional signature which includes, e.g., genes connected to TGF-β signaling and TGF-β-dependent processes, e.g., wound healing or angiogenesis (Hugo et al. Cell. 2016; 165(1):35-44). In some embodiments, TGF-β blockade extends the therapeutic window of a therapy that inhibits the PD-1/PD-L1 axis. TGF-β inhibitors can affect the clinical benefits of PD-1 immunotherapy, e.g., by modulating tumor microenvironment, e.g., vasculogenesis, fibrosis, or factors that affect the recruitment of effector T cells (Yang et al. Trends Immunol. 2010; 31(6):220-7; Wakefield and Hill Nat Rev Cancer. 2013; 13(5):328-41; Truty and Urrutia Pancreatology. 2007; 7(5-6):423-35).

Without wishing to be bound by theory, it is also believed that in some embodiments, a number of elements of the anti-tumor immunity cycle express both PD-1 and TGF-β receptors, and PD-1 and TGF-β receptors are likely to propagate non-redundant cellular signals. For example, in mouse models of autochthonous prostate cancer, the use of either a dominant-negative form of TGFBRII, or abrogation of TGF-β production in T cells delays tumor growth (Donkor et al. Immunity. 2011; 35(1):123-34; Diener et al. Lab Invest. 2009; 89(2):142-51). Studies in the transgenic adenocarcinoma of the mouse prostate (TRAMP) mice showed that blocking TGF-β signaling in adoptively transferred T cells increases their persistence and antitumor activity (Chou et al. J Immunol. 2012; 189(8):3936-46). The antitumor activity of the transferred T cells may decrease over time, partially due to PD-1 upregulation in tumor-infiltrating lymphocytes, supporting a combination of PD-1 and TGF-β inhibition as described herein. The use of neutralizing antibodies against either PD-1 or TGF-β can also affect Tregs, given their high expression levels of PD-1 and their responsiveness to TGF-β stimulation (Riella et al. Am J Transplant. 2012; 12(10):2575-87), supporting a combination of PD-1 and TGF-β inhibition to treat cancer, e.g., by enhancing the modulation of Tregs differentiation and function.

Without wishing to be bound by theory, it is believed that cancers can use TGF-β to escape immune surveillance to facilitate tumor growth and metastatic progression. For example, in certain advanced cancers, high levels of TGF-β are associated with tumor aggressiveness and poor prognosis, and TGF-β pathway can promote one or more of cancer cell motility, invasion, EMT, or a stem cell phenotype. Immune regulation mediated by cancer cells and leukocyte populations (e.g., through a variety of cell-expressed or secreted molecules, e.g., IL-10 or TGF-β) may limit the response to checkpoint inhibitors as monotherapy in certain patients. In certain embodiments, a combined inhibition of TGF-β with a checkpoint inhibitor (e.g., an inhibitor of PD-1 described herein) is used to treat a cancer that does not respond, or responds poorly, to a checkpoint inhibitor (e.g., anti-PD-1) monotherapy, e.g., a pancreatic cancer or a colorectal cancer (e.g., a microsatellite stable colorectal cancer (MSS-CRC)). In other embodiments, a combined inhibition of TGF-β with a checkpoint inhibitor (e.g., an inhibitor of PD-1 described herein) is used to treat a cancer that shows a high level of effector T cell infiltration, e.g., a lung cancer (e.g., a non-small cell lung cancer), a breast cancer (e.g., a triple negative breast cancer), a liver cancer (e.g., a hepatocellular carcinoma), a prostate cancer, or a renal cancer (e.g., a clear cell renal cell carcinoma). In some embodiments, the combination of a TGF-β inhibitor and an inhibitor of PD-1 may result in a synergistic effect.

In one embodiment, the TGF-β inhibitor (e.g., XOMA 089) is administered at a dose between 0.1 mg/kg and 20 mg/kg, e.g., between 0.1 mg/kg and 15 mg/kg, between 0.1 mg/kg and 12 mg/kg, between 0.3 mg/kg and 6 mg/kg, between 1 mg/kg and 3 mg/kg, between 0.1 mg/kg and 1 mg/kg, between 0.1 mg/kg and 0.5 mg/kg, between 0.1 mg/kg and 0.3 mg/kg, between 0.3 mg/kg and 3 mg/kg, between 0.3 mg/kg and 1 mg/kg, between 3 mg/kg and 6 mg/kg, or between 6 mg/kg and 12 mg/kg, e.g., at a dose of about 0.1 mg/kg, 0.3 mg/kg, 0.5 mg/kg, 1 mg/kg, 3 mg/kg, 6 mg/kg, 12 mg/kg, or 15 mg/kg, e.g., once every week, once every two weeks, once every three weeks, once every four weeks, or once every six weeks.

In one embodiment, the TGF-β inhibitor (e.g., XOMA 089) is administered at a dose between 0.1 mg/kg and 15 mg/kg (e.g., between 0.3 mg/kg and 12 mg/kg or between 1 mg/kg and 6 mg, e.g., about 0.1 mg/kg, 0.3 mg/kg, 1 mg/kg, 3 mg/kg, 6 mg/kg, 12 mg/kg, or 15 mg/kg), e.g., once every three weeks. For example, the TGF-β inhibitor (e.g., XOMA 089) can be administered at a dose between 0.1 mg/kg and 1 mg/kg (e.g., between 0.1 mg/kg and 1 mg/kg, e.g., 0.3 mg/kg), e.g., once every three weeks. In one embodiment, the TGF-β inhibitor (e.g., XOMA 089) is administered intravenously.

In some embodiments, the TGF-β inhibitor is administered in combination with a PD-1 inhibitor (e.g., an anti-PD-1 antibody molecule) and along with a PSMA therapeutic agent, such as radiolabeled Compound I, described herein.

In one embodiment, the TGF-β inhibitor (e.g., XOMA 089) is administered at a dose between 0.1 mg/kg and 15 mg/kg (e.g., between 0.3 mg/kg and 12 mg/kg or between 1 mg/kg and 6 mg, e.g., about 0.1 mg/kg, 0.3 mg/kg, 1 mg/kg, 3 mg/kg, 6 mg/kg, 12 mg/kg, or 15 mg/kg), e.g., once every three weeks, e.g., intravenously, and the PD-1 inhibitor (e.g., the anti-PD-1 antibody molecule) is administered at a dose between 50 mg and 500 mg (e.g., between 100 mg and 400 mg, e.g., at a dose of about 100 mg, 200 mg, 300 mg, or 400 mg), e.g., once every 3 weeks or once every 4 weeks, e.g., by intravenous infusion. In some embodiments, the PD-1 inhibitor (e.g., the anti-PD-1 antibody molecule) is administered at a dose between 100 mg and 300 mg (e.g., at a dose of about 100 mg, 200 mg, or 300 mg), e.g., once every 3 weeks, e.g., by intravenous infusion.

In some embodiments, the TGF-β inhibitor (e.g., XOMA 089) is administered at a dose of about 0.1 mg/kg or 0.3 mg/kg, e.g., once every 3 weeks, e.g., by intravenous infusion, and the PD-1 inhibitor (e.g., the anti-PD-1 antibody molecule) is administered at a dose of about 100 mg, e.g., once every 3 weeks, e.g., by intravenous infusion. In some embodiments, the TGF-β inhibitor (e.g., XOMA 089) is administered at a dose of about 0.3 mg/kg, e.g., once every 3 weeks, e.g., by intravenous infusion, and the PD-1 inhibitor (e.g., the anti-PD-1 antibody molecule) is administered at a dose of about 100 mg or 300 mg, e.g., once every 3 weeks, e.g., by intravenous infusion. In some embodiments, the TGF-β inhibitor (e.g., XOMA 089) is administered at a dose of about 1 mg/kg, 3 mg/kg, 6 mg/kg, 12 mg/kg, or 15 mg/kg, e.g., once every 3 weeks, e.g., by intravenous infusion, and the PD-1 inhibitor (e.g., the anti-PD-1 antibody molecule) is administered at a dose of about 300 mg, e.g., once every 3 weeks, e.g., by intravenous infusion.

In some embodiments, the TGF-β inhibitor (e.g., XOMA 089) is administered at a dose between 0.1 mg and 0.2 mg (e.g., about 0.1 mg/kg), e.g., once every three weeks, e.g., by intravenous infusion, and the PD-1 inhibitor (e.g., the anti-PD-1 antibody molecule) is administered at a dose between 50 mg and 200 mg (e.g., about 100 mg), e.g., once every three weeks, e.g., by intravenous infusion.

In some embodiments, the TGF-β inhibitor (e.g., XOMA 089) is administered at a dose between 0.2 mg and 0.5 mg (e.g., about 0.3 mg/kg), e.g., once every three weeks, e.g., by intravenous infusion, and the PD-1 inhibitor (e.g., the anti-PD-1 antibody molecule) is administered at a dose between 50 mg and 200 mg (e.g., about 100 mg), e.g., once every three weeks, e.g., by intravenous infusion.

In some embodiments, the TGF-β inhibitor (e.g., XOMA 089) is administered at a dose between 0.2 mg and 0.5 mg (e.g., about 0.3 mg/kg), e.g., once every three weeks, e.g., by intravenous infusion, and the PD-1 inhibitor (e.g., the anti-PD-1 antibody molecule) is administered at a between 200 mg and 400 mg (e.g., about 300 mg), e.g., once every three weeks, e.g., by intravenous infusion.

In some embodiments, the TGF-β inhibitor (e.g., XOMA 089) is administered at a dose between 0.5 mg and 2 mg (e.g., about 1 mg/kg), e.g., once every three weeks, e.g., by intravenous infusion, and the PD-1 inhibitor (e.g., the anti-PD-1 antibody molecule) is administered at a between 200 mg and 400 mg (e.g., about 300 mg), e.g., once every three weeks, e.g., by intravenous infusion.

In some embodiments, the TGF-β inhibitor (e.g., XOMA 089) is administered at a dose between 2 mg and 5 mg (e.g., about 3 mg/kg), e.g., once every three weeks, e.g., by intravenous infusion, and the PD-1 inhibitor (e.g., the anti-PD-1 antibody molecule) is administered at a between 200 mg and 400 mg (e.g., about 300 mg), e.g., once every three weeks, e.g., by intravenous infusion.

In some embodiments, the TGF-β inhibitor (e.g., XOMA 089) is administered at a dose between 5 mg and 10 mg (e.g., about 6 mg/kg), e.g., once every three weeks, e.g., by intravenous infusion, and the PD-1 inhibitor (e.g., the anti-PD-1 antibody molecule) is administered at a between 200 mg and 400 mg (e.g., about 300 mg), e.g., once every three weeks, e.g., by intravenous infusion.

In some embodiments, the TGF-β inhibitor (e.g., XOMA 089) is administered at a dose between 10 mg and 15 mg (e.g., about 12 mg/kg), e.g., once every three weeks, e.g., by intravenous infusion, and the PD-1 inhibitor (e.g., the anti-PD-1 antibody molecule) is administered at a between 200 mg and 400 mg (e.g., about 300 mg), e.g., once every three weeks, e.g., by intravenous infusion.

In some embodiments, the TGF-β inhibitor (e.g., XOMA 089) is administered at a dose between 10 mg and 20 mg (e.g., about 15 mg/kg), e.g., once every three weeks, e.g., by intravenous infusion, and the PD-1 inhibitor (e.g., the anti-PD-1 antibody molecule) is administered at a between 200 mg and 400 mg (e.g., about 300 mg), e.g., once every three weeks, e.g., by intravenous infusion.

In some embodiments, the TGF-β inhibitor (e.g., XOMA 089) is administered before the PD-1 inhibitor (e.g., the anti-PD-1 antibody molecule) is administered. In other embodiments, the TGF-β inhibitor (e.g., XOMA 089) is administered after the PD-1 inhibitor (e.g., the anti-PD-1 antibody molecule) is administered. In certain embodiments, the TGF-β inhibitor (e.g., XOMA 089) and the PD-1 inhibitor (e.g., the anti-PD-1 antibody molecule), are administered separately with at least a 30-minute (e.g., at least 1, 1.5, or 2 hours) break between the two administrations.

In some embodiments, the combination or method comprises a PD-1 inhibitor (e.g., a PD-1 inhibitor described herein), a TGF-β inhibitor (e.g., a TGF-β inhibitor described herein) and one or more of a MEK inhibitor (e.g., a MEK inhibitor described herein), an IL-1 inhibitor (e.g., a IL-1b inhibitor described herein) or an A2aR antagonist (e.g., an A2aR antagonist described herein), along with the PSMA therapeutic agent, such as radiolabeled Compound I described herein. Without wishing to be bound by theory, it is believed that in some embodiments TGFβ facilitates immunosuppression by Treg subsets in CRC and pancreatic cancer. In some embodiments, the combination or method comprising a PD-1 inhibitor, a TGF-β inhibitor, and one or more of a MEK inhibitor, an IL-1b inhibitor or an A2aR antagonist, along with a PSMA therapeutic agent, such as radiolabeled Compound I described herein, is administered in a therapeutically effective amount to a subject, e.g., with CRC or pancreatic cancer or prostate cancer.

In some embodiments, a combination or method comprising a PD-1 inhibitor (e.g., a PD-1 inhibitor described herein), and a TGF-β inhibitor (e.g., a TGF-β inhibitor described herein), along with a PSMA therapeutic agent, such as radiolabeled Compound I described herein, may show improved efficacy in controlling tumor growth in a murine MC38 CRC model compared to any agent alone. Without wishing to be bound by theory, it is believed that in some embodiments a TGF-β inhibitor in combination with a PD-1 inhibitor improves, e.g., increases, the efficacy of the PD-1 inhibitor. In some embodiments, a combination or method comprising a PD-1 inhibitor (e.g., a PD-1 inhibitor described herein), and a TGF-β inhibitor (e.g., a TGF-β inhibitor described herein) administered to a subject with, e.g., a CRC, may result in an improved, e.g., increased, efficacy of the PD-1 inhibitor.

### Other Exemplary TGF-β Inhibitors

In some embodiments, the TGF-β inhibitor comprises fresolimumab (CAS Registry Number: 948564-73-6). Fresolimumab is also known as GC1008. Fresolimumab is a human monoclonal antibody that binds to and inhibits TGF-beta isoforms 1, 2 and 3.

The heavy chain of fresolimumab has the amino acid
sequence of:
(SEQ ID NO: 160)
QVQLVQSGAEVKKPGSSVKVSCKASGYTFSSNVISWVRQAPGQGLEWMGGV
IPIVDIANYAQRFKGRVTITADESTSTTYMELSSLRSEDTAVYYCASTLGL
VLDAMDYWGQGTLVTVSSASTKGPSVFPLAPCSRSTSESTAALGCLVKDYF
PEPVTVSWNSGALTSGVHTFPAVLQSSGLYSLSSVVTVPSSSLGTKTYTCN
VDHKPSNTKVDKRVESKYGPPCPSCPAPEFLGGPSVFLFPPKPKDTLMISR
TPEVTCVVVDVSQEDPEVQFNWYVDGVEVHNAKTKPREEQFNSTYRVVSVL
TVLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLYS
RLTVDKSRWQEGNVFSCSVMHEALHNHYTQKSLSLSLGK.
The light chain of fresolimumab has the amino acid
sequence of:
(SEQ ID NO: 161)
ETVLTQSPGTLSLSPGERATLSCRASQSLGSSYLAWYQQKPGQAPRLLIYG
ASSRAPGIPDRFSGSGSGTDFTLTISRLEPEDFAVYYCQQYADSPITFGQG
TRLEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSS
PVTKSFNRGEC.

Fresolimumab is disclosed, e.g., in International Application Publication No. WO 2006/086469, and U.S. Pat. Nos. 8,383,780 and 8,591,901, which are incorporated herein by reference in their entirety.

### IL-15/IL-15Ra Complexes

In certain embodiments, the combination or method described herein comprises an IL-15/IL-15Ra complex as I-O therapeutic agent. In some embodiments, the IL-15/IL-15Ra complex is chosen from NIZ985 (Novartis), ATL-803 (Altor) or CYP0150 (Cytune). In some embodiments, the IL-15/IL-15RA complex is NIZ985. Without wishing to be bound by theory, it is believed that in some embodiments, IL-15 potentiates, e.g., enhances, Natural Killer cells to eliminate, e.g., kill, pancreatic cancer cells. In an embodiment, response, e.g., therapeutic response, to a combination or method described herein, e.g., a combination or method comprising an IL-15/IL15Ra complex, in, e.g., an animal model of colorectal cancer is associated with Natural Killer cell infiltration.

### Exemplary IL-15/IL-15Ra Complexes

In one embodiment, the IL-15/IL-15Ra complex comprises human IL-15 complexed with a soluble form of human IL-15Ra. In one aspect, the complex may comprise IL-15 covalently or noncovalently bound to a soluble form of IL-15Ra. In one embodiment, the human IL-15 is noncovalently bonded to a soluble form of IL-15Ra. In another embodiment, the human IL-15 of the composition comprises an amino acid sequence of SEQ ID NO: 164 in Table 9 and the soluble form of human IL-15Ra comprises an amino acid sequence of SEQ ID NO:165 in Table 9, as described in WO 2014/066527, incorporated herein by reference in its entirety. The molecules described herein can be made by vectors, host cells, and methods described in WO 2007/084342, incorporated herein by reference in its entirety.

TABLE 9
Amino acid and nucleotide sequences of exemplary
IL-15/IL-15Ra complexes
NIZ985

| | | |
|---|---|---|
| SEQ ID | Human IL-15 | NWVNVISDLKKIEDLIQSMHIDATLYTESD |
| NO: 164 | | VHPSCKVTAMKCFLLELQVISLESGDASIH |
| | | DTVENLIILANNSLSSNGNVTESGCKECEE |
| | | LEEKNIKEFLQSFVHIVQMFINTS |
| SEQ ID | Human | ITCPPPMSVEHADIWVKSYSLYSRERYICN |
| NO: 165 | Soluble IL- | SGFKRKAGTSSLTECVLNKATNVAHWTTPS |
| | 15Ra | LKCIRDPALVHQRPAPPSTVTTAGVTPQPE |
| | | SLSPSGKEPAASSPSSNNTAATTAAIVPGS |
| | | QLMPSKSPSTGTTEISSHESSHGTPSQTTA |
| | | KNWELTASASHQPPGVYPQG |

Without wishing to be bound by theory, it is believed that in microsatellite stable CRCs with low T cell infiltration, IL-15 may promote, e.g., increase, T cell priming (e.g., as described in Lou, K. J. SciBX 7(16); 10.1038/SCIBX.2014.449). In some embodiments, the combination or method comprises a PD-1 inhibitor (e.g., a PD-1 inhibitor described herein), an IL-15/IL15RA complex (e.g., an IL-15/IL15RA complex described herein) and one or more of a MEK inhibitor (e.g., a MEK inhibitor described herein), an IL-1b inhibitor (e.g., a IL-1b inhibitor described herein) or an A2aR antagonist (e.g., an A2aR antagonist described herein), along with a PSMA therapeutic agent, such as radiolabeled Compound I described herein. In some embodiments, the combination or method promotes, e.g., increases T cell priming. Without wishing to be bound by theory, it is further believed that IL-15 may induce NK cell infiltration. In some embodiments, response to a PD-1 inhibitor, an IL-15/IL-15RA complex and one or more of a MEK inhibitor, an IL-1b inhibitor, or an A2Ar antagonist, along with a PSMA therapeutic agent, such as radiolabeled Compound I described herein, may result in NK cell infiltration.

### Other Exemplary IL-15/IL-15Ra Complexes

In one embodiment, the IL-15/IL-15Ra complex is ALT-803, an IL-15/IL-15Ra Fc fusion protein (IL-15N72D:IL-15RaSu/Fc soluble complex). ALT-803 is disclosed in WO 2008/143794, incorporated herein by reference in its entirety. In one embodiment, the IL-15/IL-15Ra Fc fusion protein comprises the sequences as disclosed in Table 10.

In one embodiment, the IL-15/IL-15Ra complex comprises IL-15 fused to the sushi domain of IL-15Ra (CYP0150, Cytune). The sushi domain of IL-15Ra refers to a domain beginning at the first cysteine residue after the signal peptide of IL-15Ra, and ending at the fourth cysteine residue after said signal peptide. The complex of IL-15 fused to the sushi domain of IL-15Ra is disclosed in WO 2007/04606 and WO 2012/175222, incorporated herein by reference in their entirety. In one embodiment, the IL-15/IL-15Ra sushi domain fusion comprises the sequences as disclosed in Table 10.

TABLE 10
Amino acid sequences of other exemplary IL-15/IL-15Ra complexes
ALT-803 (Altor)

| | | |
|---|---|---|
| SEQ ID | IL-15N72D | NWVNVISDLKKIEDLIQSMHIDATLYTESDVHPSCKV |
| NO: 166 | | TAMKCFLLELQVISLESGDASIHDTVENLIILANDSLS |
| | | TS |
| SEQ ID | IL-15RaSu/Fc | ITCPPPMSVEHADIWVKSYSLYSRERYICNSGFKRKA |
| NO: 167 | | GTSSLTECVLNKATNVAHWTTPSLKCIREPKSCDKT |
| | | HTCPPCPAPELLGGPSVFLFPPKPKDTLMISRTPEVTC |
| | | VVVDVSHEDPEVKFNWYVDGVEVHNAKTKPREEQ |
| | | YNSTYRVVSVLTVLHQDWLNGKEYKCKVSNKALPA |
| | | PIEKTISKAKGQPREPQVYTLPPSRDELTKNQVSLTCL |
| | | VKGFYPSDIAVEWESNGQPENNYKTTPPVLDSDGSF |
| | | FLYSKLTVDKSRWQQGNVFSCSVMHEALHNHYTQK |
| | | SLSLSPGK |

IL-15/IL-15Ra sushi domain fusion (Cytune)

| | | |
|---|---|---|
| SEQ ID | Human IL-15 | NWVNVISDLKKIEDLIQSMHIDATLYTESDVHPSCKV |
| NO: 168 | | TAMKCFLLELQVISLESGDASIHDTVENLIILANNSLS |
| | | SNGNVTESGCKECEELEEKNIKEFLQSFVHIVQMFIN |
| | | TS |
| SEQ ID | | NWVNVISDLKKIEDLIQSMHIDATLYTESDVHPSCKV |
| NO: 169 | | TAMKCFLLELQVISLESGDASIHDTVENLIILANNSLS |
| | | SNGNVTESGCKECEELEKKNIKEFLQSFVHIVQMFIN |
| | | TS |
| SEQ ID | Human IL-15Ra | ITCPPPMSVEHADIWVKSYSLYSRERYICNSGFKRKA |
| NO: 170 | sushi and hinge | GTSSLTECVLNKATNVAHWTTPSLKCIRDPALVHQR |
| | domains | PAPP |

### Further Anti-Cancer Agents

In certain instances, the combination according to the invention comprising the PSMA therapeutic agent, such as radiolabeled Compound I are combined with additional other therapeutic agents, such as other anti-cancer agents, anti-allergic agents, anti-nausea agents (or anti-emetics), chemotherapeutic agents, pain relievers, cytoprotective agents, and combinations thereof.

In other embodiments, anti-cancer agents that can be combined with the PSMA therapeutic agent, such as radiolabeled Compound of the Formula I, II, or IIa, described herein include:

Tyrosine kinase inhibitors: Erlotinib hydrochloride (Tarceva®); Linifanib (N-[4-(3-amino-1H-indazol-4-yl)phenyl]-N′-(2-fluoro-5-methylphenyl)urea, also known as ABT 869, available from Genentech); Sunitinib malate (Sutent®); Bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, and described in U.S. Pat. No. 6,780,996); Dasatinib (Sprycel®); Pazopanib (Votrient®); Sorafenib (Nexavar®); Zactima (ZD6474); and Imatinib or Imatinib mesylate (Gilvec® and Gleevec®).

Vascular Endothelial Growth Factor (VEGF) receptor inhibitors: Bevacizumab (Avastin®), axitinib (Inlyta®); Brivanib alaninate (BMS-582664, (S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TKI258, CAS 852433-84-2); Linifanib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); and Aflibercept (Eylea®), sulfatinib, surufatinib.

Platelet-derived Growth Factor (PDGF) receptor inhibitors: Imatinib (Gleevec®); Linifanib (N-[4-(3-amino-1H-indazol-4-yl)phenyl]-N′-(2-fluoro-5-methylphenyl)urea, also known as ABT 869, available from Genentech); Sunitinib malate (Sutent®); Quizartinib (AC220, CAS 950769-58-1); Pazopanib (Votrient®); Axitinib (Inlyta®); Sorafenib (Nexavar®); Vargatef (BIBF1120, CAS 928326-83-4); Telatinib (BAY57-9352, CAS 332012-40-5); Vatalanib dihydrochloride (PTK787, CAS 212141-51-0); and Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470).

Fibroblast Growth Factor Receptor (FGFR) Inhibitors: Brivanib alaninate (BMS-582664, (S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); Vargatef (BIBF1120, CAS 928326-83-4); Dovitinib dilactic acid (TKI258, CAS 852433-84-2); 3-(2,6-Dichloro-3,5-dimethoxy-phenyl)-1-{6-[4-(4-ethyl-piperazin-1-yl)-phenylamino]-pyrimidin-4-yl}-1-methyl-urea (BGJ398, CAS 872511-34-7); Danusertib (PHA-739358); N-[2-[[4-(Diethylamino)butyl]amino]-6-(3,5-dimethoxyphenyl)pyrido[2,3-d]pyrimidin-7-yl]-N′-(1,1-dimethylethyl)-urea (PD173074, CAS 219580-11-7); sulfatinib; and surufatinib.

Aurora kinase inhibitors: Danusertib (PHA-739358); N-[4-[[6-Methoxy-7-[3-(4-morpholinyl)propoxy]-4-quinazolinyl]amino]phenyl]benzamide (ZM447439, CAS 331771-20-1); 4-(2-Amino-4-methyl-5-thiazolyl)-N-[4-(4-morpholinyl)phenyl]-2-pyrimidinamine (CYC116, CAS 693228-63-6); Tozasertib (VX680 or MK-0457, CAS 639089-54-6); Alisertib (MLN8237); (N-{2-[6-(4-Cyclobutylamino-5-trifluoromethyl-pyrimidine-2-ylamino)-(1S,4R)-1,2,3,4-tetrahydro-1,4-epiazano-naphthalen-9-yl]-2-oxo-ethyl}-acetamide) (PF-03814735); 4-[[9-Chloro-7-(2,6-difluorophenyl)-5H-pyrimido[5,4-d][2]benzazepin-2-yl]amino]-benzoic acid (MLN8054, CAS 869363-13-3); Cenisertib (R-763); Barasertib (AZD1152); and N-cyclopropyl-N′-[3-[6-(4-morpholinylmethyl)-1H-benzimidazol-2-yl]-1H-pyrazol-4-yl]-urea (AT9283).

Cyclin-Dependent Kinase (CDK) inhibitors: Aloisine A; Alvocidib (also known as flavopiridol or HMR-1275, 2-(2-chlorophenyl)-5,7-dihydroxy-8-[(3S,4R)-3-hydroxy-1-methyl-4-piperidinyl]-4-chromenone, and described in U.S. Pat. No. 5,621,002); Crizotinib (PF-02341066, CAS 877399-52-5); 2-(2-Chlorophenyl)-5,7-dihydroxy-8-[(2R,3S)-2-(hydroxymethyl)-1-methyl-3-pyrrolidinyl]-4H-1-benzopyran-4-one, hydrochloride (P276-00, CAS 920113-03-7); Indisulam (E7070); Roscovitine (CYC202); 6-Acetyl-8-cyclopentyl-5-methyl-2-(5-piperazin-1-yl-pyridin-2-ylamino)-8H-pyrido[2,3-d]pyrimidin-7-one, hydrochloride (PD0332991); Dinaciclib (SCH727965); N-[5-[[(5-tert-Butyloxazol-2-yl)methyl]thio]thiazol-2-yl]piperidine-4-carboxamide (BMS 387032, CAS 345627-80-7); 4-[[9-Chloro-7-(2,6-difluorophenyl)-5H-pyrimido[5,4-d][2]benzazepin-2-yl]amino]-benzoic acid (MLN8054, CAS 869363-13-3); 5-[3-(4,6-Difluoro-1H-benzimidazol-2-yl)-1H-indazol-5-yl]-N-ethyl-4-methyl-3-pyridinemethanamine (AG-024322, CAS 837364-57-5); 4-(2,6-Dichlorobenzoylamino)-1H-pyrazole-3-carboxylic acid N-(piperidin-4-yl)amide (AT7519, CAS 844442-38-2); 4-[2-Methyl-1-(1-methylethyl)-1H-imidazol-5-yl]-N-[4-(methylsulfonyl)phenyl]-2-pyrimidinamine (AZD5438, CAS 602306-29-6); Palbociclib (PD-0332991); and (2R,3R)-3-[[2-[[3-[[S(R)]—S-cyclopropylsulfonimidoyl]-phenyl]amino]-5-(trifluoromethyl)-4-pyrimidinyl]oxy]-2-butanol (BAY 10000394), ribociclib.

Checkpoint Kinase (CHK) inhibitors: 7-Hydroxystaurosporine (UCN-01); 6-Bromo-3-(1-methyl-1H-pyrazol-4-yl)-5-(3R)-3-piperidinyl-pyrazolo[1,5-a]pyrimidin-7-amine (SCH900776, CAS 891494-63-6); 5-(3-Fluorophenyl)-3-ureidothiophene-2-carboxylic acid N—[(S)-piperidin-3-yl]amide (AZD7762, CAS 860352-01-8); 4-[((3S)-1-Azabicyclo[2.2.2]oct-3-yl)amino]-3-(1H-benzimidazol-2-yl)-6-chloroquinolin-2(1H)-one (CHIR 124, CAS 405168-58-3); 7-Aminodactinomycin (7-AAD), Isogranulatimide, debromohymenialdisine; N-[5-Bromo-4-methyl-2-[(2S)-2-morpholinylmethoxy]-phenyl]-N′-(5-methyl-2-pyrazinyl)urea (LY2603618, CAS 911222-45-2); Sulforaphane (CAS 4478-93-7,4-Methylsulfinylbutyl isothiocyanate); 9,10,11,12-Tetrahydro-9,12-epoxy-1H-diindolo[1,2,3-fg:3′,2′,1′-kl]pyrrolo[3,4-i][1,6]benzodiazocine-1,3(2H)-dione (SB-218078, CAS 135897-06-2); and TAT-S216A (YGRKKRRQRRRLYRSPAMPENL), and CBP501 ((d-Bpa)sws(d-Phe-F5)(d-Cha)rrrqrr); and (αR)-α-amino-N-[5,6-dihydro-2-(1-methyl-1H-pyrazol-4-yl)-6-oxo-1H-pyrrolo[4,3,2-ef][2,3]benzodiazepin-8-yl]-Cyclohexaneacetamide (PF-0477736).

3-Phosphoinositide-dependent kinase-1 (PDK1 or PDPK1) inhibitors: 7-2-Amino-N-[4-[5-(2-phenanthrenyl)-3-(trifluoromethyl)-1H-pyrazol-1-yl]phenyl]-acetamide (OSU-03012, CAS 742112-33-0); Pyrrolidine-1-carboxylic acid (3-{5-bromo-4-[2-(1H-imidazol-4-yl)-ethylamino]-pyrimidin-2-ylamino}-phenyl)-amide (BX912, CAS 702674-56-4); and 4-Dodecyl-N-1,3,4-thiadiazol-2-yl-benzenesulfonamide (PHT-427, CAS 1191951-57-1).

Pyruvate Dehydrogenase Kinase (PDK) inhibitors: (+)-Dehydroabietylamine; Dichloroacetic acid (DCA); and Leelamine.

Protein Kinase B (PKB) or AKT inhibitors: 8-[4-(1-Aminocyclobutyl)phenyl]-9-phenyl-1,2,4-triazolo[3,4-f][1,6]naphthyridin-3(2H)-one (MK-2206, CAS 1032349-93-1); Perifosine (KRX0401); 4-Dodecyl-N-1,3,4-thiadiazol-2-yl-benzenesulfonamide (PHT-427, CAS 1191951-57-1); 4-[2-(4-Amino-1,2,5-oxadiazol-3-yl)-1-ethyl-7-[(3S)-3-piperidinylmethoxy]-1H-imidazo[4,5-c]pyridin-4-yl]-2-methyl-3-butyn-2-ol (GSK690693, CAS 937174-76-0); 8-(1-Hydroxyethyl)-2-methoxy-3-[(4-methoxyphenyl)methoxy]-6H-dibenzo[b,d]pyran-6-one (palomid 529, P529, or SG-00529); Triciribine (6-Amino-4-methyl-8-(β-D-ribofuranosyl)-4H,8H-pyrrolo[4,3,2-de]pyrimido[4,5-c]pyridazine); (αS)-α-[[[5-(3-Methyl-1H-indazol-5-yl)-3-pyridinyl]oxy]methyl]-benzeneethanamine (A674563, CAS 552325-73-2); 4-[(4-Chlorophenyl)methyl]-1-(7H-pyrrolo[2,3-d]pyrimidin-4-yl)-4-piperidinamine (CCT128930, CAS 885499-61-6); 4-(4-Chlorophenyl)-4-[4-(1H pyrazol-4-yl)phenyl]-piperidine (AT7867, CAS 857531-00-1); and Archexin (RX-0201, CAS 663232-27-7)

Protein Kinase C (PKC) activators: Bryostatin I (bryo-1) and Sotrastaurin (AEB071).

B-RAF inhibitors: Regorafenib (BAY73-4506, CAS 755037-03-7); Tuvizanib (AV951, CAS 475108-18-0); Vemurafenib (Zelboraf®, PLX-4032, CAS 918504-65-1); 5-[1-(2-Hydroxyethyl)-3-(pyridin-4-yl)-1H-pyrazol-4-yl]-2,3-dihydroinden-1-one oxime (GDC-0879, CAS 905281-76-7); 5-[2-[4-[2-(Dimethylamino)ethoxy]phenyl]-5-(4-pyridinyl)-1H-imidazol-4-yl]-2,3-dihydro-1H-Inden-1-one oxime (GSK2118436 or SB590885); (+/−)-Methyl (5-(2-(5-chloro-2-methylphenyl)-1-hydroxy-3-oxo-2,3-dihydro-1H-isoindol-1-yl)-1H-benzimidazol-2-yl)carbamate (also known as XL-281 and BMS908662) and N-(3-(5-chloro-1H-pyrrolo[2,3-b]pyridine-3-carbonyl)-2,4-difluorophenyl)propane-1-sulfonamide (also known as PLX4720).

C-RAF Inhibitors: Sorafenib (Nexavar®); 3-(Dimethylamino)-N-[3-[(4-hydroxybenzoyl)amino]-4-methylphenyl]-benzamide (ZM336372, CAS 208260-29-1); and 3-(1-cyano-1-methylethyl)-N-[3-[(3,4-dihydro-3-methyl-4-oxo-6-quinazolinyl)amino]-4-methylphenyl]-benzamide (AZ628, CAS 1007871-84-2).

Human Granulocyte colony-stimulating factor (G-CSF) modulators: Filgrastim (Neupogen®); Sunitinib malate (Sutent®); Pegfilgrastim (Neulasta®) and Quizartinib (AC220, CAS 950769-58-1).

RET Inhibitors: Sunitinib malate (Sutent®); Vandetanib (Caprelsa®); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Sorafenib (BAY 43-9006); Regorafenib (BAY73-4506, CAS 755037-03-7); and Danusertib (PHA-739358).

FMS-like Tyrosine kinase 3 (FLT3) Inhibitors or CD135: Sunitinib malate (Sutent®); Quizartinib (AC220, CAS 950769-58-1); N-[(1-Methyl-4-piperidinyl)methyl]-3-[3-(trifluoromethoxy)phenyl]-Imidazo[1,2-b]pyridazin-6-amine sulfate (SGI-1776, CAS 1173928-26-1); and Vargatef (BIBF1120, CAS 928326-83-4).

c-KIT Inhibitors: Pazopanib (Votrient®); Dovitinib dilactic acid (TKI258, CAS 852433-84-2); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Masitinib (Masivet®); Regorafenib (BAY73-4506, CAS 755037-03-7); Tivozanib (AV951, CAS 475108-18-0); Vatalanib dihydrochloride (PTK787, CAS 212141-51-0); Telatinib (BAY57-9352, CAS 332012-40-5); Foretinib (GSK1363089, formerly XL880, CAS 849217-64-7); Sunitinib malate (Sutent®); Quizartinib (AC220, CAS 950769-58-1); Axitinib (Inlyta®); Dasatinib (BMS-345825); and Sorafenib (Nexavar®).

Bcr/Abl kinase inhibitors: Imatinib (Gleevec®); Inilotinib hydrochloride; Nilotinib (Tasigna®); Dasatinib (BMS-345825); Bosutinib (SKI-606); Ponatinib (AP24534); Bafetinib (INNO406); Danusertib (PHA-739358), AT9283 (CAS 1133385-83-7); Saracatinib (AZD0530); and N-[2-[(1S,4R)-6-[[4-(Cyclobutylamino)-5-(trifluoromethyl)-2-pyrimidinyl]amino]-1,2,3,4-tetrahydronaphthalen-1,4-imin-9-yl]-2-oxoethyl]-acetamide (PF-03814735, CAS 942487-16-3).

IGF-1R inhibitors: Linsitnib (OSI-906); [7-[trans-3-[(Azetidin-1-yl)methyl]cyclobutyl]-5-(3-benzyloxyphenyl)-7H-pyrrolo[2,3-d]pyrimidin-4-yl]amine (AEW541, CAS 475488-34-7); [5-(3-Benzyloxyphenyl)-7-[trans-3-[(pyrrolidin-1-yl)methyl]cyclobutyl]-7H-pyrrolo[2,3-d]pyrimidin-4-yl]amine (ADW742 or GSK552602A, CAS 475488-23-4); (2-[[3-Bromo-5-(1,1-dimethylethyl)-4-hydroxyphenyl]methylene]-propanedinitrile (Tyrphostin AG1024, CAS 65678-07-1); 4-[[(2S)-2-(3-Chlorophenyl)-2-hydroxyethyl]amino]-3-[7-methyl-5-(4-morpholinyl)-1H-benzimidazol-2-yl]-2(1H)-pyridinone (BMS536924, CAS 468740-43-4); 4-[2-[4-[[(2S)-2-(3-Chlorophenyl)-2-hydroxyethyl]amino]-1,2-dihydro-2-oxo-3-pyridinyl]-7-methyl-1H-benzimidazol-5-yl]-1-piperazinepropanenitrile (BMS554417, CAS 468741-42-6); (2S)-1-[4-[(5-Cyclopropyl-1H-pyrazol-3-yl)amino]pyrrolo[2,1-f][1,2,4]triazin-2-yl]-N-(6-fluoro-3-pyridinyl)-2-methyl-2-pyrrolidinecarboxamide (BMS754807, CAS 1001350-96-4); Picropodophyllotoxin (AXL1717); and Nordihydroguareacetic acid.

IGF-1R antibodies: Figitumumab (CP751871); Cixutumumab (IMC-A12); Ganitumab (AMG-479); Robatumumab (SCH-717454); Dalotuzumab (MK0646); R1507 (available from Roche); BIIB022 (available from Biogen); and MEDI-573 (available from MedImmune).

PIM Kinase inhibitors: 1,10-Dihydropyrrolo[2,3-a]carbazole-3-carbaldehyde (DHPCC-9); N-[(1-Methyl-4-piperidinyl)methyl]-3-[3-(trifluoromethoxy)phenyl]-Imidazo[1,2-b]pyridazin-6-amine sulfate (SGI-1776, CAS 1173928-26-1); and CX-6258 (described in ACS Med. Chem. Lett., 2012, 3 (2), pp 135-139).

MET inhibitors: Cabozantinib (XL184, CAS 849217-68-1); Foretinib (GSK1363089, formerly XL880, CAS 849217-64-7); Tivantinib (ARQ197, CAS 1000873-98-2); 1-(2-Hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide (AMG 458); Cryzotinib (Xalkori®, PF-02341066); (3Z)-5-(2,3-Dihydro-1H-indol-1-ylsulfonyl)-3-({3,5-dimethyl-4-[(4-methylpiperazin-1-yl)carbonyl]-1H-pyrrol-2-yl}methylene)-1,3-dihydro-2H-indol-2-one (SU11271); (3Z)—N-(3-Chlorophenyl)-3-({3,5-dimethyl-4-[(4-methylpiperazin-1-yl)carbonyl]-1H-pyrrol-2-yl}methylene)-N-methyl-2-oxoindoline-5-sulfonamide (SU11274); (3Z)—N-(3-Chlorophenyl)-3-{[3,5-dimethyl-4-(3-morpholin-4-ylpropyl)-1H-pyrrol-2-yl]methylene}-N-methyl-2-oxoindoline-5-sulfonamide (SU11606); 6-[Difluoro[6-(1-methyl-1H-pyrazol-4-yl)-1,2,4-triazolo[4,3-b]pyridazin-3-yl]methyl]-quinoline (JNJ38877605, CAS 943540-75-8); 2-[4-[1-(Quinolin-6-ylmethyl)-1H-[1,2,3]triazolo[4,5-b]pyrazin-6-yl]-1H-pyrazol-1-yl]ethanol (PF04217903, CAS 956905-27-4); N-((2R)-1,4-Dioxan-2-ylmethyl)-N-methyl-N′-[3-(1-methyl-1H-pyrazol-4-yl)-5-oxo-5H-benzo[4,5]cyclohepta[1,2-b]pyridin-7-yl]sulfamide (MK2461, CAS 917879-39-1); 6-[[6-(1-Methyl-1H-pyrazol-4-yl)-1,2,4-triazolo[4,3-b]pyridazin-3-yl]thio]-quinoline (SGX523, CAS 1022150-57-7); and (3Z)-5-[[(2,6-Dichlorophenyl)methyl]sulfonyl]-3-[[3,5-dimethyl-4-[[(2R)-2-(1-pyrrolidinylmethyl)-1-pyrrolidinyl]carbonyl]-1H-pyrrol-2-yl]methylene]-1,3-dihydro-2H-indol-2-one (PHA665752, CAS 477575-56-7).

Human Epidermal Growth Factor Receptor 2 (HER2 receptor) (also known as Neu, ErbB-2, CD340, or p185 inhibitors): Trastuzumab (Herceptin®); Pertuzumab (Omnitarg®); Neratinib (HKI-272, (2E)-N-[4-[[3-chloro-4-[(pyridin-2-yl)methoxy]phenyl]amino]-3-cyano-7-ethoxyquinolin-6-yl]-4-(dimethylamino)but-2-enamide, and described PCT Publication No. WO 05/028443); Lapatinib or Lapatinib ditosylate (Tykerb®); (3R,4R)-4-amino-1-((4-((3-methoxyphenyl)amino)pyrrolo[2,1-f][1,2,4]triazin-5-yl)methyl)piperidin-3-ol (BMS690514); (2E)-N-[4-[(3-Chloro-4-fluorophenyl)amino]-7-[[(3S)-tetrahydro-3-furanyl]oxy]-6-quinazolinyl]-4-(dimethylamino)-2-butenamide (BIBW-2992, CAS 850140-72-6); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS 599626, CAS 714971-09-2); Canertinib dihydrochloride (PD183805 or CI-1033); and N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8).

Epidermal growth factor receptor (EGFR) inhibitors: Erlotinib hydrochloride (Tarceva®), Gefitinib (Iressa®); N-[4-[(3-Chloro-4-fluorophenyl)amino]-7-[[(3“S”)-tetrahydro-3-furanyl]oxy]-6-quinazolinyl]-4(dimethylamino)-2-butenamide, Tovok®); Vandetanib (Caprelsa®); Lapatinib (Tykerb®); (3R,4R)-4-Amino-1-((4-((3-methoxyphenyl)amino)pyrrolo[2,1-f][1,2,4]triazin-5-yl)methyl)piperidin-3-ol (BMS690514); Canertinib dihydrochloride (CI-1033); 6-[4-[(4-Ethyl-1-piperazinyl)methyl]phenyl]-N-[(1R)-1-phenylethyl]-7H-Pyrrolo[2,3-d]pyrimidin-4-amine (AEE788, CAS 497839-62-0); Mubritinib (TAK165); Pelitinib (EKB569); Afatinib (BIBW2992); Neratinib (HKI-272); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS599626); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); and 4-[4-[[(1R)-1-Phenylethyl]amino]-7H-pyrrolo[2,3-d]pyrimidin-6-yl]-phenol (PKI166, CAS 187724-61-4).

EGFR antibodies: Cetuximab (Erbitux®); Panitumumab (Vectibix®); Matuzumab (EMD-72000); Trastuzumab (Herceptin®); Nimotuzumab (hR3); Zalutumumab; TheraCIM h-R3; MDX0447 (CAS 339151-96-1); and ch806 (mAb-806, CAS 946414-09-1).

Hedgehog antagonists: Vismodegib (2-chloro-N-[4-chloro-3-(2-pyridinyl)phenyl]-4-(methylsulfonyl)-benzamide, GDC-0449, and described in PCT Publication No. WO 06/028958); 1-(4-Chloro-3-(trifluoromethyl)phenyl)-3-((3-(4-fluorophenyl)-3,4-dihydro-4-oxo-2-quinazolinyl)methyl)-urea (CAS 330796-24-2); N-[(2S,3R,3′R,3aS,4′aR,6S,6′aR,6′bS,7aR,12′aS,12′bS)-2′,3′,3a,4,4′,4′a,5,5′,6,6′,6′a,6′b,7,7′,7a,8′,10′,12′,12′a,12′b-Eicosahydro-3,6,11′,12′b-tetramethylspiro[furo[3,2-b]pyridine-2(3H),9′(1′H)-naphth[2,1-a]azulen]-3′-yl]-methanesulfonamide (IPI926, CAS 1037210-93-7); and 4-Fluoro-N-methyl-N-[1-[4-(1-methyl-1H-pyrazol-5-yl)-1-phthalazinyl]-4-piperidinyl]-2-(trifluoromethyl)-benzamide (LY2940680, CAS 1258861-20-9).

mTOR inhibitors: Temsirolimus (Torisel®); Ridaforolimus (formally known as deforolimus, (1R,2R,4S)-4-[(2R)-2 [(1R,9S,12S,15R,16E,18R,19R,21R, 23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl dimethylphosphinate, also known as AP23573 and MK8669, and described in PCT Publication No. WO 03/064383); Everolimus (Afinitor® or RAD001); Rapamycin (AY22989, Sirolimus®); Simapimod (CAS 164301-51-3); (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-, inner salt (SF1126, CAS 936487-67-1); and N-[4-[[[3-[(3,5-dimethoxyphenyl)amino]-2-quinoxalinyl]amino]sulfonyl]phenyl]-3-methoxy-4-methyl-benzamide (XL765, also known as SAR245409); and (1r,4r)-4-(4-amino-5-(7-methoxy-1H-indol-2-yl)imidazo[1,5-f][1,2,4]triazin-7-yl)cyclohexanecarboxylic acid (OSI-027).

Phosphoinositide 3-kinase (PI3K) inhibitors: 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730); 2-Methyl-2-[4-[3-methyl-2-oxo-8-(quinolin-3-yl)-2,3-dihydroimidazo[4,5-c]quinolin-1-yl]phenyl]propionitrile (also known as BEZ235 or NVP-BEZ 235, and described in PCT Publication No. WO 06/122806); 4-(trifluoromethyl)-5-(2,6-dimorpholinopyrimidin-4-yl)pyridin-2-amine (also known as BKM120 or NVP-BKM120, and described in PCT Publication No. WO2007/084786); Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione (PX866, CAS 502632-66-8); 8-Phenyl-2-(morpholin-4-yl)-chromen-4-one (LY294002, CAS 154447-36-6); 2-Amino-8-ethyl-4-methyl-6-(1H-pyrazol-5-yl)pyrido[23-d]pyrimidin-7(8H)-one (SAR 245409 or XL 765); 5-Fluoro-3-phenyl-2-[(1S)-1-(9H-purin-6-ylamino)ethyl]-4(3H)-quinazolinone (CAL101); 2-Amino-N-[3-[N-[3-[(2-chloro-5-methoxyphenyl)amino]quinoxalin-2-yl]sulfamoyl]phenyl]-2-methylpropanamide (SAR 245408 or XL 147); and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (BYL719).

BCL-2 inhibitors: 4-[4-[[2-(4-Chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (also known as ABT-263 and described in PCT Publication No. WO 09/155386); Tetrocarcin A; Antimycin; Gossypol ((−)BL-193); Obatoclax; Ethyl-2-amino-6-cyclopentyl-4-(1-cyano-2-ethoxy-2-oxoethyl)-4Hchromone-3-carboxylate (HA14-1); Oblimersen (G3139, Genasense®); Bak BH3 peptide; (−)-Gossypol acetic acid (AT-101); 4-[4-[(4′-Chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-benzamide (ABT-737, CAS 852808-04-9); and Navitoclax (ABT-263, CAS 923564-51-6).

Mitogen-activated protein kinase (MEK) inhibitors: XL-518 (also known as GDC-0973, Cas No. 1029872-29-4, available from ACC Corp.); Selumetinib (5-[(4-bromo-2-chlorophenyl)amino]-4-fluoro-N-(2-hydroxyethoxy)-1-methyl-1H-benzimidazole-6-carboxamide, also known as AZD6244 or ARRY 142886, described in PCT Publication No. WO2003077914); 2-[(2-Chloro-4-iodophenyl)amino]-N-(cyclopropylmethoxy)-3,4-difluoro-benzamide (also known as CI-1040 or PD184352 and described in PCT Publication No. WO2000035436); N-[(2R)-2,3-Dihydroxypropoxy]-3,4-difluoro-2-[(2-fluoro-4-iodophenyl)amino]-benzamide (also known as PD0325901 and described in PCT Publication No. WO2002006213); 2,3-Bis[amino[(2-aminophenyl)thio]methylene]-butanedinitrile (also known as U0126 and described in U.S. Pat. No. 2,779,780); N-[3,4-Difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766 and described in PCT Publication No. WO2007014011); (3S,4R,5Z,8S,9S,11E)-14-(Ethylamino)-8,9,16-trihydroxy-3,4-dimethyl-3,4,9,19-tetrahydro-1H-2-benzoxacyclotetradecine-1,7(8H)-dione] (also known as E6201 and described in PCT Publication No. WO2003076424); 2′-Amino-3′-methoxyflavone (also known as PD98059 available from Biaffin GmbH & Co., KG, Germany); Vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); Pimasertib (AS-703026, CAS 1204531-26-9); Trametinib dimethyl sulfoxide (GSK-1120212, CAS 1204531-25-80); 2-(2-Fluoro-4-iodophenylamino)-N-(2-hydroxyethoxy)-1,5-dimethyl-6-oxo-1,6-dihydropyridine-3-carboxamide (AZD 8330); and 3,4-Difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655)

P38 MAPK inhibitors: Orantinib (TSU-68, CAS 252916-29-3); Dilmapimod (SB681323, CAS 444606-18-2); 6-[(Aminocarbonyl)(2,6-difluorophenyl)amino]-2-(2,4-difluorophenyl)-3-pyridinecarboxamide (VX702); 8-Phenyl-2-(morpholin-4-yl)-chromen-4-one (LY294002, CAS 154447-36-6); 4-[4-(4-fluorophenyl)-2-[4-(methylsulfinyl)phenyl]-1H-imidazol-5-yl]-pyridine (SB203580, CAS 152121-47-6); 4-[4-(4-Fluorophenyl)-2-[4-(methylsulfinyl)phenyl]-1H-imidazol-5-yl]-pyridine (SB203580, CAS 152121-47-6); trans-4-[4-(4-Fluorophenyl)-5-(2-methoxy-4-pyrimidinyl)-1H-imidazol-1-yl]-cyclohexanol (SB 239063, CAS 193551-21-2); 6-(4-Fluorophenyl)-2,3-dihydro-5-(4-pyridinyl)-imidazo[2,1-b]thiazole (SKF 86002, CAS 72873-74-6); 5-(2,6-dichlorophenyl)-2-[(2,4-difluorophenyl)thio]-6H-pyrimido[1,6-b]pyridazin-6-one (VX745, CAS 209410-46-8); Talmapimod (SCIO469, CAS 309913-83-5); 1-[4-[3-(4-chlorophenyl)-4-(4-pyrimidinyl)-1H-pyrazol-5-yl]-1-piperidinyl]-2-hydroxy-ethanone (SD0006, CAS 1184301-42-5); Dilmapimod (SB681323, CAS 444606-18-2); 3-Bromo-4-[(2,4-difluorobenzyl)oxy]-1-[5-[(methylamino)carbonyl]-2-methylphenyl]-6-methylpyridin-2(1H)-one (PH797804, CAS 586379-66-0); 4-[[5-[(Cyclopropylamino)carbonyl]-2-methylphenyl]amino]-5-methyl-N-propyl-pyrrolo[2,1-f][1,2,4]triazine-6-carboxamide (BMS-582949, CAS 623152-17-0); Pamapimod (R1503, CAS 449811-01-2); 2-[(Hexahydrocyclopenta[c]pyrrol-2(1H)-yl)amino]-8-methyl-6-(2-methylphenyl)-pyrido[2,3-d]pyrimidin-7(8H)-one (AW814141, CAS 905285-51-0); 4-[5-(4-Fluorophenyl)-2-(methylthio)-1H-imidazol-4-yl]-N-(1-phenylethyl)-2-pyridinamine, (9CI) (ML 3403); and rel-6-Chloro-5-[[(2R,5S)-4-[(4-fluorophenyl)methyl]-2,5-dimethyl-1-piperazinyl]carbonyl]-N,N,1-trimethyl-α-oxo-1H-Indole-3-acetamide (SCIO 282 and SD 282).

JAK inhibitors: Ruxolitinib (Jakafi®); Tofacitinib (CP690550); Axitinib (AG013736, CAS 319460-85-0); 5-Chloro-N²-[(1S)-1-(5-fluoro-2-pyrimidinyl)ethyl]-N⁴-(5-methyl-1H-pyrazol-3-y)-12,4-pyrimidinediamine (AZD1480, CAS 935666-88-9); and (9E)-15-[2-(1-Pyrrolidinyl)ethoxy]-7,12,26-Trioxa-19,21,24-triazatetracyclo[18.3.1.1^{2,5}.1^{14,18}]-hexacosa-1(24), 2,4,9,14,16,18(25), 20,22-nonaene (SB-1578, CAS 937273-04-6); Momelotinib (CYT 387); Baricitinib (INCB-028050 or LY-3009104); Pacritinib (SB1518); (16E)-14-Methyl-20-Oxa-5,7,14,27-tetraazatetracyclo[19.3.1.12,6.18,12]heptacosa-1(25),2,4,6(27),8,10,12(26),16,21,23-decaene (SB 1317); Gandotinib (LY 2784544); N,N-Cicyclopropyl-4-[(1,5-dimethyl-1H-pyrazol-3-yl)amino]-6-ethyl-1,6-dihydro-1-methyl-imidazo[4,5-d]pyrrolo[2,3-b]pyridine-7-carboxamide (BMS 911543); Alkylating agents: Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednimustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoramide, TESPA and TSPA, Thioplex®); Cyclophosphamide (Endoxan®, Cytoxan®, Neosar®, Procytox®, Revimmune®); and Bendamustine HCl (Treanda®).

Aromatase inhibitors: Exemestane (Aromasin®); Letrozole (Femara®); and Anastrozole (Arimidex®).

Topoisomerase I inhibitors: Irinotecan (Camptosar®); Topotecan hydrochloride (Hycamtin®); and 7-Ethyl-10-hydroxycamptothecin (SN38).

Topoisomerase II inhibitors: Etoposide (VP-16 and Etoposide phosphate, Toposar®, VePesid® and Etopophos®); Teniposide (VM-26, Vumon®); and Tafluposide.

DNA Synthesis inhibitors: Capecitabine (Xeloda®); Gemcitabine hydrochloride (Gemzar®); Nelarabine ((2R,3S,4R,5R)-2-(2-amino-6-methoxy-purin-9-yl)-5-(hydroxymethyl)oxolane-3,4-diol, Arranon® and Atriance®); and Sapacitabine (1-(2-cyano-2-deoxy-β-D-arabinofuranosyl)-4-(palmitoylamino)pyrimidin-2(1H)-one).

Folate Antagonists or Antifolates: Trimetrexate glucuronate (Neutrexin®); Piritrexim isethionate (BW201U); Pemetrexed (LY231514); Raltitrexed (Tomudex®); and Methotrexate (Rheumatrex®, Trexal®).

Immunomodulators: Afutuzumab (available from Roche®); Pegfilgrastim (Neulasta®); Lenalidomide (CC-5013, Revlimid®); Thalidomide (Thalomid®), Actimid (CC4047); and IRX-2 (mixture of human cytokines including interleukin 1, interleukin 2, and interferon 7, CAS 951209-71-5, available from IRX Therapeutics).

Proapoptotic receptor agonists (PARAs) including DR4 (TRAILR1) and DR5 (TRAILR2): Dulanermin (AMG-951, RhApo2L/TRAIL); Mapatumumab (HRS-ETR1, CAS 658052-09-6); Lexatumumab (HGS-ETR2, CAS 845816-02-6); Apomab (Apomab®); Conatumumab (AMG655, CAS 896731-82-1); and Tigatuzumab (CS1008, CAS 946415-34-5, available from Daiichi Sankyo).

Phospholipase A2 (PLA₂) inhibitors: Manoalide; E-(3-Acetamide-1-benzyl-2-ethylindolyl-5-oxy)propane sulfonic acid (LY311727); Anagrelide (Agrylin®); Methyl arachidonoyl fluorophosphonate (MAFP); Arachidonoyl trifluoromethyl ketone (AACOCF₃); (E)-6-(1-bromoethyl)tetrahydro-3-(1-naphthalenyl)-2H-pyran-2-one (Bromoenol lactone or BEL); R-Bromoenol lactone (R-BEL); S-Bromoenol lactone (S-BEL); Diisopropylfluorophosphate (DFP); Phenylmethylsulfonylfluoride (PMSF); and Pefabloc (CAS 34284-75-8, 4-[2-aminoethyl]benzenesulfonyl fluoride).

SRC inhibitors: Dasatinib (Sprycel®); Saracatinib (AZD0530, CAS 379231-04-6); Bosutinib (SKI-606, CAS 380843-75-4); 5-[4-[2-(4-Morpholinyl)ethoxy]phenyl]-N-(phenylmethyl)-2-pyridineacetamide (KX2-391, CAS 897016-82-9); and 4-(2-Chloro-5-methoxyanilino)-6-methoxy-7-(1-methylpiperidin-4-ylmethoxy)quinazoline (AZM475271, CAS 476159-98-5).

Osteoclastic bone resorption inhibitors: Zoledronate (Zometa®); Ibandronate (Boniva®); Alendronate (Fosamax®); Risedronate (Actonel®, Atelvia®, and Benet®); and Mineral trioxide aggregate (MTA).

G-Protein-coupled Somatostatin receptors Inhibitors: Octreotide (also known as octreotide acetate, Sandostatin® and Sandostatin LAR®); Lanreotide acetate (CAS 127984-74-1); Seglitide (MK678); Vapreotide acetate (Sanvar®); and Cyclo(D-Trp-Lys-Abu-Phe-MeAla-Tyr)(BIM23027).

Interleukin-11 and Synthetic Interleukin-11 (IL-11): Oprelvekin (Neumega®).

Cell growth stimulator: Palifermin (Kepivance®), Erythropoietin (Epogen® and Procrit®); Darbepoetin alfa (Aranesp®); Peginesatide (Hematide®); and EPO covalently linked to polyethylene glycol (Micera®).

Receptor Activator for Nuclear Factor κ B (RANK) inhibitors: Denosumab (Prolia® and Xgeva®).

Thrombopoietin mimetic peptibodies: Romiplostim (Nplate®).

Histone deacetylase (HDAC) inhibitors: Voninostat (Zolinza®); Romidepsin (Istodax®); Treichostatin A (TSA); Oxamflatin; Vorinostat (Zolinza®, Suberoylanilide hydroxamic acid); Pyroxamide (syberoyl-3-aminopyridineamide hydroxamic acid); Trapoxin A (RF-1023A); Trapoxin B (RF-10238); Cyclo[(αS,2S)-α-amino-η-oxo-2-oxiraneoctanoyl-O-methyl-D-tyrosyl-L-isoleucyl-L-prolyl] (Cy1-1); Cyclo[(αS,2S)-α-amino-η-oxo-2-oxiraneoctanoyl-O-methyl-D-tyrosyl-L-isoleucyl-(2S)-2-piperidinecarbonyl] (Cy1-2); Cyclic[L-alanyl-D-alanyl-(2S)-η-oxo-L-α-aminooxiraneoctanoyl-D-prolyl] (HC-toxin); Cyclo[(aS,2S)-α-amino-η-oxo-2-oxiraneoctanoyl-D-phenylalanyl-L-leucyl-(2S)-2-piperidinecarbonyl] (WF-3161); Chlamydocin ((S)-Cyclic(2-methylalanyl-L-phenylalanyl-D-prolyl-η-oxo-L-α-aminooxiraneoctanoyl); Apicidin (Cyclo(8-oxo-L-2-aminodecanoyl-1-methoxy-L-tryptophyl-L-isoleucyl-D-2-piperidinecarbonyl); Romidepsin (Istodax®, FR-901228); 4-Phenylbutyrate; Spiruchostatin A; Mylproin (Valproic acid); Entinostat (MS-275, N-(2-Aminophenyl)-4-[N-(pyridine-3-yl-methoxycarbonyl)-amino-methyl]-benzamide); and Depudecin (4,5:8,9-dianhydro-1,2,6,7,11-pentadeoxy-D-threo-D-ido-Undeca-1,6-dienitol).

Biologic response modifiers: Include therapeutics such as interferons, interleukins, colony-stimulating factors, monoclonal antibodies, vaccines (therapeutic and prophylactic), gene therapy, and nonspecific immunomodulating agents. Interferon alpha (Intron®, Roferson®-A); Interferon beta; Interferon gamma; Interleukin-2 (IL-2 or aldesleukin, Proleukin®); Filgrastim (Neupogen®); Sargramostim (Leukine®); Erythropoietin (epoetin); Interleukin-11 (oprelvekin); Imiquimod (Aldara®); Lenalidomide (Revlimid®); Rituximab (Rituxan®); Trastuzumab (Herceptin®); Bacillus calmette-guerin (theraCys® and TICE® BCG); Levamisole (Ergamisol®); and Denileukin diftitox (Ontak®).

Anti-tumor antibiotics: Doxorubicin (Adriamycin® and Rubex®); Bleomycin (Lenoxane®); Daunorubicin (daunorubicin hydrochloride, daunomycin, and rubidomycin hydrochloride, Cerubidine®); Daunorubicin liposomal (daunorubicin citrate liposome, DaunoXome®); Mitoxantrone (DHAD, Novantrone®); Epirubicin (Ellence™); Idarubicin (Idamycin®, Idamycin PFS®); Mitomycin C (Mutamycin®); Geldanamycin; Herbimycin; Ravidomycin; and Desacetylravidomycin.

Anti-microtubule or Anti-mitotic agents: Vinca Alkaloids (such as Vinorelbine tartrate (Navelbine®), Vincristine (Oncovin®), and Vindesine (Eldisine®)); Taxanes (such as paclitaxel and docetaxel); and Estramustine (Emcyl® or Estracyt®); Plant Alkaloids: Paclitaxel (Taxol and Onxal™); Paclitaxel protein-bound (Abraxane®); Vinblastine (also known as vinblastine sulfate, vincaleukoblastine and VLB, Alkaban-AQ® and Velban®); Vincristine (also known as vincristine sulfate, LCR, and VCR, Oncovin® and Vincasar Pfs®); and Vinorelbine (Navelbine®).

Taxane anti-neoplastic agents: Paclitaxel (Taxol®); Docetaxel (Taxotere®); Cabazitaxel (Jevtana®, 1-hydroxy-7β,10β-dimethoxy-9-oxo-5β,20-epoxytax-11-ene-2α,4,13α-triyl-4-acetate-2-benzoate-13-[(2R,3S)-3-{[(tert-butoxy)carbonyl]amino}-2-hydroxy-3-phenylpropanoate); and Larotaxel ((2α,3ξ,4α,5β,7α,10β,13α)-4,10-bis(acetyloxy)-13-({(2R,3S)-3-[(tert-butoxycarbonyl)amino]-2-hydroxy-3-phenylpropanoyl}oxy)-1-hydroxy-9-oxo-5,20-epoxy-7,19-cyclotax-11-en-2-yl benzoate).

Cathepsin K inhibitors: Odanacatib (MK-0822, N-(1-cyanocyclopropyl)-4-fluoro-N2-{(1S)-2,2,2-trifluoro-1-[4′-(methylsulfonyl)biphenyl-4-yl]ethyl}-L-leucinamide and described in PCT Publication no. WO 03/075836); Balicatib (N-(1-((Cyanomethyl)carbamoyl)cyclohexyl)-4-(4-propylpiperazin-1-yl)benzamide, AAE581, CAS 354813-19-7); and Relacatib (SB-462795, CAS 362505-84-8).

Epothilone B analogs: Ixabepilone (Lxempra®); Patupilone (EP0906); Sagopilone (CAS 305841-29-6); and 21-Aminoepothilone B (BMS-310705, CAS 280578-49-6).

Heat Shock Protein (HSP) inhibitors: Tanespimycin (17-allylamino-17-demethoxygeldanamycin, also known as KOS-953 and 17-AAG, available from SIGMA, and described in U.S. Pat. No. 4,261,989); Retaspimycin (IPI504), GaPETespib (STA-9090); [6-Chloro-9-(4-methoxy-3,5-dimethylpyridin-2-ylmethyl)-9H-purin-2-yl]amine (BIIB021 or CNF2024, CAS 848695-25-0); trans-4-[[2-(Aminocarbonyl)-5-[4,5,6,7-tetrahydro-6,6-dimethyl-4-oxo-3-trifluoromethyl)-1H-indazol-1-yl]phenyl]amino]cyclohexyl glycine ester (SNX5422 or PF04929113, CAS 908115-27-5); and 17-Dimethylaminoethylamino-17-demethoxygeldanamycin (17-DMAG).

Farnesyl Transferase Inhibitors (FTI): Tipifarnib (R115777, Zarnestra®); Lonafarnib (SCH66336); [2S-[1[R*(R*)],2R*(S*),3R*]]-2-[[2-[[2-[(2-Amino-3-mercaptopropyl)amino]-3-methylpentyl]oxy]-1-oxo-3-phenylpropyl]amino]-4-(methylsulfonyl)-butanoic acid, 1-methylethyl ester (L-744832, CAS 160141-09-3); and (R)-2,3,4,5-Tetrahydro-1-(1H-imidazol-4-ylmethyl)-3-(phenylmethyl)-4-(2-thienylsulfonyl)-1H-1,4-benzodiazepine-7-carbonitrile (BMS-214662, CAS 195987-41-8).

Thrombopoietin (TpoR) agonists: Eltrombopag (SB497115, Promacta® and Revolade®); and Romiplostim (Nplate®).

Proteosome inhibitors: Bortezomib (Velcade®); Ixazomib citrate (MLN9708, CAS 1201902-80-8); Carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); Marizomib (NPI-0052); Danoprevir (RG7227, CAS 850876-88-9); Ixazomib (MLN2238, CAS 1072833-77-2); and (S)—N-[(phenylmethoxy)carbonyl]-L-leucyl-N-(1-formyl-3-methylbutyl)-L-Leucinamide (MG-132, CAS 133407-82-6); Delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

Kinesis Spindle Protein (KSP) inhibitors (also known as Eg5 inhibitors): Monastrol (Ethyl 4-(3-hydroxyphenyl)-6-methyl-2-sulfanylidene-3,4-dihydro-1H-pyrimidine-5-carboxylate); Ispinesib (SB715992); (2S)-4-(2,5-Difluorophenyl)-N-[(3R,4S)-3-fluoro-1-methyl-4-piperidinyl]-2,5-dihydro-2-(hydroxymethyl)-N-methyl-2-phenyl-1H-pyrrole-1-carboxamide (MK-0731, CAS 845256-65-7); Litronesib (LY2523355, CAS 910634-41-2); and (2S)-2-(3-Aminopropyl)-5-(2,5-difluorophenyl)-N-methoxy-N-methyl-2-phenyl-1,3,4-thiadiazole-3(2H)-carboxamide (ARRY520); and 9-Cyclopentyl-7,9-dihydro-2-[[2-methoxy-4-[(1-methyl-4-piperidinyl)oxy]phenyl]amino]-7-methyl-8H-purin-8-one (AZ 3146).

Polo-like kinase (Plk) inhibitors: (R)-4-[(8-Cyclopentyl-7-ethyl-5,6,7,8-tetrahydro-5-methyl-6-oxo-2-pteridinyl)amino]-3-methoxy-N-(1-methyl-4-piperidinyl)benzamide (B12536, CAS 755038-02-9); Wortmannin; Morin; Quercetin; Volasertib (BI6727); 8-Phenyl-2-(morpholin-4-yl)-chromen-4-one (LY294002); 5-[6-[(4-Methylpiperazin-1-yl)methyl]-1H-benzimidazol-1-yl]-3-[[(1R)-1-[2-(trifluoromethyl)phenyl]ethyl]oxy]-thiophene-2-carboxamide (GSK461364); (E)-4-[2-[2-[N-Acetyl-N-[(p-methoxyphenyl)sulfonyl]amino]phenyl]ethenyl]pyridine 1-oxide (HMN214); and Rigosertib (ON 01910).

Adrenal steroid inhibitors: Aminoglutethimide (Cytadren®); Trilostane (Modrenal® or Vetoryl®); and Mitotane (Lysodren®).

Anti-androgens: Nilutamide (Nilandron® and Anandron®); Bicalutamide (Casodex®); Megestrol (Megace®); Cyproterone acetate (Cyprostat®, Androcur®, or Cyproterone®), and Flutamide (Fulexin™ or Eulexin®); Leuprolide (Lupron®, Viadur® or Eligard®); Foserelin (Zoladex®); Triptorelin (Trelstar Depot®); Abarelix (Plenaxis®); and Finasteride (Andozac® or MK-906).

Anabolic Steroids: Fluoxymesterone (Halotestin®); Oxymetholone (Anadrol 50®); Oxandrolone (Oxandrin)®; and Stanozolol (Winstrol®).

Gonadotropin-releasing hormone (GnRH) receptor agonists: Leuprolide or leuprolide acetate (Viadure®, Eligard® and Lupron®); Buserelin (Suprefact® or Suprecor®); Nafarelin (Synarel®); Histrelin (Supprelin LA® or Vantas®); Goserelin (Zoladex®); Deslorelin (Suprelorin® or Ovuplant®); Degarelix (Firmagon®); and Triptorelin (Decapeptyl®, Diphereline®, Goapeptyl®, Trelstart® or Variopeptyl® 0.1).

HPV vaccines: Human papilloma virus (HPV) vaccine (Cervarix® (ATC code J07BM02), and Gardasil® (ATC code J07BM01).

Iron Chelating agents: Silybin; Curcumin; Ethylene diamine tetraacetic acid (EDTA); Triapine (3-aminopyridine-2-carboxaldehyde thiosemicarbazone); Di-2-pyridylketone thiosemicarbazone; Di-2-pyridylketone-4,4,-dimethyl-3-thiosemicarbazone; Desferrioxamine; and Deferasinox (Exjade®).

Anti-metabolites: Claribine (2-chlorodeoxyadenosine, Leustatin®); 5-Fluorouracil (Adrucil®); 6-Thioguanine (Purinethol®); Pemetrexed (Alimta®); Cytarabine (also known as arabinosylcytosine (Ara-C), Cytosar-U®); Cytarabine liposomal (also known as Liposomal Ara-C, DepoCyt™); Decitabine (Dacogen®); Hydroxyurea (Hydrea®, Droxia™ and Mylocel™); Fludarabine (Fludara®); Floxuridine (FUDR®); Methotrexate (also known as amethopterin, methotrexate sodim (MTX); Rheumatrex® and Trexall™); Pentostatin (Nipent®); Raltitrexed (Tomudex®); and Pralatrexate (Folotyn™).

Bisphosphonates: Pamidronate (Aredia®); Zoledronic acid or Zoledronate (Zometa®, Zomera®, Aclasta®, or Reclast®); Alendronate (Fosamax®); Risedronate (Actonel®); and Ibandronate (Boniva®).

Demethylating agents: 5-Azacitidine (Vidaza®); and Decitabine (Dacogen®).

Retinoids: Alitretinoin (9-cis-retinoic acid, Panretin®); Tretinoin (all-trans retinoic acid, also known as ATRA, Vesanoid®); Isotretinoin (13-cis-retinoic acid, Accutane®, Amnesteem®, Claravis®, Clarus®, Decutan®, Isotane®, Izotech®, Oratane®, Isotret®, and Sotret®); Bexarotene (Targretin®), Liposomal retinoic acid; Tazarotene (Tazorac®, Avage® or Zorac®); all-trans retinol; all-trans retinaldehyde (also known as all-trans retinal); all-trans 4-oxo retinoic acid; retinyl palmitate; and retinyl acetate.

Cytokines: Interleukin-2 (also known as aldesleukin and IL-2, Proleukin®); Interleukin-11 (also known as oprelvekin, Neumega®); and Alpha interferon alfa (also known as IFN-alpha, Intron® A, and Roferon-A®).

Estrogen receptor downregulators: Fulvestrant (Faslodex®).

Anti-estrogens: Tamoxifen (Novaldex®); Toremifene (Fareston®); and Fulvestrant (Faslodex®).

Selective estrogen receptor modulators (SERMs): Raloxifene (Evista®); Bazedoxifene; Tamoxifen (Nolvadex®); and Toremifene (Fareston®).

Leutinizing hormone releasing hormone (LHRH) agonists: Goserelin (Zoladex®); and Leuprolide acetate (Eligard® or Lupron®).

Progesterones: Megestrol (also known as megestrol acetate, Megace®).

17a-hydroxylase/C17,20 lyase (CYP17A1) inhibitors: Abiraterone acetate (Zyitga®).

Miscellaneous cytotoxic agents: Arsenic trioxide (Trisenox®); Asparaginase (also known as L-asparaginase, Erwinia L-asparaginase, Elspar® and Kidrolase®); and Asparaginase Erwinia Chrysanthemi (Erwinaze®).

C—C Chemokine receptor 4 (CCR4) Antibody: Mogamulizumab (Potelligent®)

CD20 antibodies: Rituximab (Riuxan® and MabThera®); and Tositumomab (Bexxar®); and Ofatumumab (Arzerra®).

CD20 Antibody Drug Conjugates: Ibritumomab tiuxetan (Zevalin®); and Tositumomab,

CD22 Antibody Drug Conjugates: Inotuzumab ozogamicin (also referred to as CMC-544 and WAY-207294, available from Hangzhou Sage Chemical Co., Ltd.)

CD30 mAb-cytotoxin Conjugates: Brentuximab vedotin (Adcetrix®);

CD33 Antibody Drug Conjugates: Gemtuzumab ozogamicin (Mylotarg®),

CD40 antibodies: Dacetuzumab (also known as SGN-40 or huS2C6, available from Seattle Genetics, Inc),

CD52 antibodies: Alemtuzumab (Campath®),

Anti-CS1 antibodies: Elotuzumab (HuLuc63, CAS No. 915296-00-3)

CTLA-4 antibodies: Tremelimumab (IgG2 monoclonal antibody available from Pfizer, formerly known as ticilimumab, CP-675,206); and Ipilimumab (CTLA-4 antibody, also known as MDX-010, CAS No. 477202-00-9).

TPH inhibitors: telotristat

PARP (poly ADP ribose polymerase) inhibitors: olaparib (Lynparza), rucaparib (Rubraca), Niraparib (Zeluja), Talazoparib, Veliparib. In one embodiment, the therapeutic agent administered in addition to a PSMA therapeutic agent, such as radiolabeled Compound of the Formula I, II, or IIa described herein, is not Olaparib.

Radio-sensitizers: Idronoxil (Veyonda, also known as NOX-66), Sodium glycididazole, Nimorazole, NBTXR3 (also known as PEP503), [89Zr]AGuIX, Lucanthone, Telomelysin (OBP-301), lonidamine, nimorazole, panobinostat, celecoxib, cilengitide, entinostat, etanidazole, ganetespib ((STA-9090).

In one embodiment, the present invention provides the combination or combination therapy of the PSMA therapeutic agent, such as radiolabeled Compound I, and one or more therapeutic agents selected from the group consisting of octreotide, lanreotide, vaproreotide, pasireotide, satoreotide, everolimus, temozolomide, telotristat, sunitinib, sulfatinib, ribociclib, entinostat, pazopanib, and olaparib. In one embodiment, the therapeutic agent administered in addition to a PSMA therapeutic agent, such as radiolabeled Compound I described herein, is not Olaparib.

Some patients may experience allergic reactions to the compounds of the present invention and/or other anti-cancer agent(s) during or after administration; therefore, anti-allergic agents are often administered to minimize the risk of an allergic reaction. Suitable anti-allergic agents include corticosteroids, such as dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednisol® and Solu-Medrol®); antihistamines, such as diphenhydramine (e.g., Benadryl®), hydroxyzine, and cyproheptadine; and bronchodilators, such as the beta-adrenergic receptor agonists, albuterol (e.g., Proventil®), and terbutaline (Brethine®).

Some patients may experience nausea during and after administration of the compound of the present invention and/or other anti-cancer agent(s); therefore, anti-emetics are used in preventing nausea (upper stomach) and vomiting. Suitable anti-emetics include aprepitant (Emend®), ondansetron (Zofran®), granisetron HCl (Kytril®), lorazepam (Ativan®. dexamethasone (Decadron®), prochlorperazine (Compazine®), casopitant (Rezonic® and Zunrisa®), and combinations thereof

Medication to alleviate the pain experienced during the treatment period is often prescribed to make the patient more comfortable. Common over-the-counter analgesics, such Tylenol®, are often used. However, opioid analgesic drugs such as hydrocodone/paracetamol or hydrocodone/acetaminophen (e.g., Vicodin®), morphine (e.g., Astramorph® or Avinza®), oxycodone (e.g., OxyContin® or Percocet®), oxymorphone hydrochloride (Opana®), and fentanyl (e.g., Duragesic®) are also useful for moderate or severe pain.

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy. Suitable cytoprotective agents include Amifostine (Ethyol®), glutamine, dimesna (Tavocept®), mesna (Mesnex®), dexrazoxane (Zinecard® or Totect®), xaliproden (Xaprila®), and leucovorin (also known as calcium leucovorin, citrovorum factor and folinic acid).

The structure of the active compounds identified by code numbers, generic or trade names may be taken from the actual edition of the standard compendium “The Merck Index” or from databases, e.g. Patents International (e.g. IMS World Publications).

The above-mentioned compounds, which can be used in combination with a PSMA therapeutic agent of the present invention, can be prepared and administered as described in the art, such as in the documents cited above.

In one embodiment, the present invention provides pharmaceutical compositions comprising the combination according to the invention or a pharmaceutically acceptable salt thereof together with a pharmaceutically acceptable carrier suitable for administration to a human or animal subject, either alone or together with other anti-cancer agents as previously described.

In one embodiment, the present invention provides methods of treating human or animal subjects suffering from a cellular proliferative disease, such as cancer, preferably PSMA expressing cancers

The present invention provides methods of treating a human or animal subject in need of such treatment, comprising administering to the subject a therapeutically effective amount of a combination according to the invention) or a pharmaceutically acceptable salt thereof, either alone or in combination with other anti-cancer agents.

In particular, combinations will either be formulated together as a combination therapeutic or administered separately.

In combination therapy, the compound of the present invention and other anti-cancer agent(s) may be administered either simultaneously, concurrently or sequentially with no specific time limits, wherein such administration provides therapeutically effective levels of the two compounds in the body of the patient.

In a preferred embodiment, the combination of the present invention and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The combination of the present invention and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

The combination comprising a PSMA therapeutic, such as radiolabeled Compound I described herein may also be used to advantage in combination with known therapeutic processes, for example, the administration of hormones or especially radiation. A compound of the present invention may in particular be used as a radiosensitizer, especially for the treatment of tumors which exhibit poor sensitivity to radiotherapy

### Methods of Treating Cancer

In one aspect, the disclosure relates to treatment of a subject in vivo using a combination comprising a PSMA therapeutic agent, such as radiolabeled Compound I described herein, and additional therapeutic agents disclosed herein, or a composition or formulation comprising a combination disclosed herein, such that growth of cancerous tumors is inhibited or reduced.

In some embodiments, the additional therapeutic agent is an Immuno-Oncology therapeutic agent as described above. Preferred I-O therapeutic agent(s) is (are) selected from LAG-3 inhibitors, TIM-3 inhibitors, GITR angonists, TGF-β inhibitors, IL15/IL-15RA complexes, and PD-1 inhibitors, PD-L1 inhibitors, and CTLA-4 inhibitors, wherein said PD-1 inhibitors are selected from Spartalizumab, Pembrolizumab, Pidilizumab, Durvalomab, Atezolizumab, Avelumab, Nivolumab, MK-3475, MPDL3280A, MEDI4736, ipilimumab, tremelimumab, MEDI0680, REGN2810, TSR-042, PF-06801591, BGB-A317, BGB-108, INCSHR1210, and AMP-224.

The combination according to the invention comprising a PSMA therapeutic agent, such as radiolabeled Compound I described herein and additional therapeutic agents such as an Immuno-Oncology therapeutic agent disclosed herein are administered or used in accordance with a dosage regimen disclosed herein.

In one embodiment, the combination disclosed herein is used for the treatment of cancer in vivo in a subject, particularly for the treatment of prostate cancer and other cancers that express prostate-specific membrane antigen (PSMA), e.g. PSMA expressing cancers such as PSMA over-expressing cancers and cancers that express PSMA in their neovasculature.

In another embodiment, the combination according to the invention is used for the treatment in a subject of PSMA expressing cancer selected from thyroid cancer, renal clear cell carcinoma, transitional cell carcinoma of the bladder, colonic adenocarcinoma, neuroendocrine carcinoma, glioblastoma multiforme, malignant melanoma, pancreatic duct carcinoma, non-small cell lung carcinoma, soft tissue sarcoma, and breast carcinoma

For example, the combination can be used to inhibit the growth of cancerous tumors. The combination can also be used in combination with one or more of: a standard of care treatment (e.g., for cancers or infectious disorders), a vaccine (e.g., a therapeutic cancer vaccine), a cell therapy, a radiation therapy, surgery, or any other therapeutic agent or modality, to treat a disorder herein. For example, to achieve antigen-specific enhancement of immunity, the combination can be administered together with an antigen of interest. In one embodiment, the combination disclosed herein can be administered in any order or simultaneously.

In another aspect, a method of treating a subject, e.g., reducing or ameliorating, a hyperproliferative condition or disorder (e.g., a cancer), e.g., solid tumor, a hematological cancer, soft tissue tumor, or a metastatic lesion, in a subject is provided. The method includes administering to the subject the combination comprising at least two, or two or more, or at least three or more (e.g., four or more) therapeutic agents disclosed herein, or a composition or formulation comprising a combination disclosed herein, e.g., in accordance with a dosage regimen disclosed herein.

As used herein, the term “cancer” is meant to include all types of cancerous growths or oncogenic processes, metastatic tissues or malignantly transformed cells, tissues, or organs, irrespective of histopathological type or stage of invasiveness. Examples of cancerous disorders include, but are not limited to, solid tumors, hematological cancers, soft tissue tumors, and metastatic lesions. Examples of solid tumors include malignancies, e.g., sarcomas, and carcinomas (including adenocarcinomas and squamous cell carcinomas), of the various organ systems, such as those affecting liver, lung, breast, lymphoid, thyroid, colon, the neuroendocrine system, gastrointestinal (e.g., colon), genitourinary tract (e.g., renal, urothelial, bladder cells), prostate, CNS (e.g., brain, neural or glial cells), skin (e.g., melanoma), pancreas, and pharynx. Adenocarcinomas include malignancies such as most colon cancers, rectal cancer, renal-cell carcinoma, liver cancer, non-small cell carcinoma of the lung, cancer of the small intestine and cancer of the esophagus. Squamous cell carcinomas include malignancies, e.g., in the lung, esophagus, skin, head and neck region, oral cavity, anus, and cervix. Metastatic lesions of the aforementioned cancers can also be treated or prevented using the methods, combinations, and compositions of the invention.

As used herein, the term “subject” is intended to include human and non-human animals.

In one embodiment, the combination therapies described herein can include a composition of the present invention co-formulated with, and/or co-administered with, one or more additional therapeutic agents as previously described, e.g., one or more anti-cancer agents, cytotoxic or cytostatic agents, hormone treatment, vaccines, and/or other immunotherapies as previously described. In other embodiments, the combination is further administered or used in combination with other therapeutic treatment modalities, including surgery, radiation, cryosurgery, and/or thermotherapy. In one aspect, such combination therapies may advantageously utilize lower dosages of the administered therapeutic agents, thus avoiding possible toxicities or complications associated with the various monotherapies.

In one embodiment, when administered in combination, the PSMA therapeutic agent, such as radiolabeled Compound I, or the additional therapeutic agent can be administered in an amount or dose that is higher or lower than, or the same as, the amount or dosage of each agent used individually, e.g., as a monotherapy. In certain embodiments, the administered amount or dosage of the PSMA therapeutic agent, such as radiolabeled Compound I, or the additional therapeutic agent is lower (e.g., at least 20%, at least 30%, at least 40%, or at least 50%) than the amount or dosage of each agent used individually, e.g., as a monotherapy. In other embodiments, the amount or dosage of the PSMA therapeutic agent, such as radiolabeled Compound I, or the additional therapeutic agent that results in a desired effect (e.g., treatment of cancer) is lower (e.g., at least 20%, at least 30%, at least 40%, or at least 50% lower).

### Pharmaceutical Compositions

In another aspect, the present invention provides compositions, e.g., pharmaceutically acceptable compositions, which includes one or more of, e.g., two, three, four, five, six, seven, eight, or more of, a PSMA therapeutic agent, such as radiolabeled Compound I and an additional therapeutic agent, such as the Immune-Oncology therapeutic agent formulated alone or together with a pharmaceutically acceptable carrier.

As used herein, “pharmaceutically acceptable carrier” includes any and all solvents, dispersion media, isotonic and absorption delaying agents, and the like that are physiologically compatible.

In one aspect, the carrier can be suitable for intravenous, intramuscular, subcutaneous, parenteral, rectal, spinal or epidermal administration (e.g. by injection or infusion).

The compositions described herein may be in a variety of forms.

In various embodiments, these include, for example, liquid, semi-solid and solid dosage forms, such as liquid solutions (e.g., injectable and infusible solutions), dispersions or suspensions, liposomes and suppositories. The form depends on the intended mode of administration and therapeutic application.

In one aspect, compositions are in the form of injectable or infusible solutions. In certain embodiments, the mode of administration is parenteral (e.g., intravenous, subcutaneous, intraperitoneal, or intramuscular).

In an embodiment, the composition is administered by intravenous infusion or injection. In another embodiment, the composition is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

In one aspect, therapeutic compositions should be sterile and stable under the conditions of manufacture and storage.

In various embodiments, the composition can be formulated as a solution, microemulsion, dispersion, liposome, or other ordered structure.

In one embodiment, the composition is suitable for high antibody concentration. Sterile injectable solutions can be prepared by incorporating the active Compound I and the additional therapeutic agent in the required amount in an appropriate solvent with one or a combination of ingredients enumerated above, as required, followed by filtered sterilization.

In one embodiment, dispersions are prepared by incorporating the active Compound I and the additional therapeutic agent into a sterile vehicle that contains a basic dispersion medium and the required other ingredients from those enumerated above. In the case of sterile powders for the preparation of sterile injectable solutions, suitable methods of preparation are vacuum drying and freeze-drying that yields a powder of the active ingredient plus any additional desired ingredient from a previously sterile-filtered solution thereof.

In one aspect, the proper fluidity of a solution can be maintained, for example, by the use of a coating such as lecithin, by the maintenance of the required particle size in the case of dispersion and by the use of surfactants.

In another aspect, prolonged absorption of injectable compositions can be brought about by including in the composition an agent that delays absorption, for example, monostearate salts and gelatin.

In some embodiments, a PSMA therapeutic agent, such as radiolabeled Compound I, and the additional therapeutic agent, such as the Immuno-Oncology therapeutic agent can be formulated into a formulation (e.g., a dose formulation or dosage form) suitable for administration (e.g., intravenous administration) to a subject as described herein. Preferably, the Immuno-Oncology therapeutic agent is (are) selected from PD-1 inhibitors, LAG-3 inhibitors, TIM-3 inhibitors, GITR agonists, TGF-β inhibitors, an IL-15/IL-15RA complexes, or any combination thereof.

In certain embodiments, the formulation is a drug substance formulation. In other embodiments, the formulation is a lyophilized formulation, e.g., lyophilized or dried from a drug substance formulation. In other embodiments, the formulation is a reconstituted formulation, e.g., reconstituted from a lyophilized formulation. In other embodiments, the formulation is a liquid formulation.

In some embodiments, the formulation (e.g., drug substance formulation) comprises a combination comprising a PSMA therapeutic agent, such as radiolabeled Compound I described herein, and additional therapeutic agents, such as an Immuno-Oncology therapeutic agent previously described, for example a PD-1 inhibitor, a LAG-3 inhibitor, a TIM-3 inhibitor, a GITR agonist, a TGF-β inhibitor, an IL-15/IL-15RA complex, or any combination thereof.

In some embodiments, the formulation is a drug substance formulation. In some embodiments, the formulation (e.g., drug substance formulation) comprises the combination according to the invention and a buffering agent.

In some embodiments, the formulation (e.g., drug substance formulation) comprises the combination according to the invention at a concentration of 10 to 50 mg/mL, e.g., 15 to 50 mg/mL, 20 to 45 mg/mL, 25 to 40 mg/mL, 30 to 35 mg/mL, 25 to 35 mg/mL, or 30 to 40 mg/mL, e.g., 15 mg/mL, 20 mg/mL, 25 mg/mL, 30 mg/mL, 33.3 mg/mL, 35 mg/mL, 40 mg/mL, 45 mg/mL, or 50 mg/mL. In certain embodiments, the combination according to the invention is present at a concentration of 30 to 35 mg/mL, e.g., 33.3 mg/mL.

In some embodiments, the formulation (e.g., drug substance formulation) comprises a buffering agent comprising histidine (e.g., a histidine buffer). In certain embodiments, the buffering agent (e.g., histidine buffer) is present at a concentration of 1 mM to 20 mM, e.g., 2 mM to 15 mM, 3 mM to 10 mM, 4 mM to 9 mM, 5 mM to 8 mM, or 6 mM to 7 mM, e.g., 1 mM, 2 mM, 3 mM, 4 mM, 5 mM, 6 mM, 6.7 mM, 7 mM, 8 mM, 9 mM, 10 mM, 11 mM, 12 mM, 13 mM, 14 mM, 15 mM, 16 mM, 17 mM, 18 mM, 19 mM, or 20 mM. In some embodiments, the buffering agent (e.g., histidine buffer) is present at a concentration of 6 mM to 7 mM, e.g., 6.7 mM. In other embodiments, the buffering agent (e.g., a histidine buffer) has a pH of 4 to 7, e.g., 5 to 6, e.g., 5, 5.5, or 6. In some embodiments, the buffering agent (e.g., histidine buffer) has a pH of 5 to 6, e.g., 5.5. In certain embodiments, the buffering agent comprises histidine at a concentration of 6 mM to 7 mM (e.g., 6.7 mM) and has a pH of 5 to 6 (e.g., 5.5).

In some embodiments, the formulation (e.g., drug substance formulation) comprises the combination according to the invention at a concentration of 30 to 35 mg/mL, e.g., 33.3 mg/mL; and a buffering agent that comprises histidine at a concentration of 6 mM to 7 mM (e.g., 6.7 mM) and has a pH of 5 to 6 (e.g., 5.5).

In some embodiments, the formulation (e.g., drug substance formulation) further comprises a carbohydrate. In certain embodiments, the carbohydrate is sucrose. In some embodiments, the carbohydrate (e.g., sucrose) is present at a concentration of 50 mM to 150 mM, e.g., 25 mM to 150 mM, 50 mM to 100 mM, 60 mM to 90 mM, 70 mM to 80 mM, or 70 mM to 75 mM, e.g., 25 mM, 50 mM, 60 mM, 70 mM, 73.3 mM, 80 mM, 90 mM, 100 mM, or 150 mM. In some embodiments, the formulation comprises a carbohydrate or sucrose present at a concentration of 70 mM to 75 mM, e.g., 73.3 mM.

In some embodiments, the formulation (e.g., drug substance formulation) comprises the combination according to the invention at a concentration of 30 to 35 mg/mL, e.g., 33.3 mg/mL; a buffering agent that comprises histidine at a concentration of 6 mM to 7 mM (e.g., 6.7 mM) and has a pH of 5 to 6 (e.g., 5.5); and a carbohydrate or sucrose present at a concentration of 70 mM to 75 mM, e.g., 73.3 mM.

In some embodiments, the formulation is a drug substance formulation. In some embodiments, the formulation (e.g., drug substance formulation) comprises the combination according to the invention, which comprises PSMA therapeutic agent, such as radiolabeled Compound I, and an Immuno-Oncology therapeutic agent, for example a PD-1 inhibitor, a LAG-3 inhibitor, a TIM-3 inhibitor, a GITR agonist, a TGF-β inhibitor, an IL-15/IL-15RA complex, or any combination thereof and a buffering agent.

In some embodiments, the formulation (e.g., drug substance formulation) further comprises a surfactant. In certain embodiments, the surfactant is polysorbate 20. In some embodiments, the surfactant or polysorbate 20) is present at a concentration of 0.005% to 0.025% (w/w), e.g., 0.0075% to 0.02% or 0.01% to 0.015% (w/w), e.g., 0.005%, 0.0075%, 0.01%, 0.013%, 0.015%, or 0.02% (w/w). In some embodiments, the formulation comprises a surfactant or polysorbate 20 present at a concentration of 0.01% to 0.015%, e.g., 0.013% (w/w).

In some embodiments, the formulation (e.g., drug substance formulation) the combination according to the invention at a concentration of 30 to 35 mg/mL, e.g., 33.3 mg/mL; a buffering agent that comprises histidine at a concentration of 6 mM to 7 mM (e.g., 6.7 mM) and has a pH of 5 to 6 (e.g., 5.5); and a surfactant or polysorbate 20 present at a concentration of 0.01% to 0.015%, e.g., 0.013% (w/w).

In some embodiments, the formulation (e.g., drug substance formulation) comprises the combination according to the invention at a concentration of 30 to 35 mg/mL, e.g., 33.3 mg/mL; a buffering agent that comprises histidine at a concentration of 6 mM to 7 mM (e.g., 6.7 mM) and has a pH of 5 to 6 (e.g., 5.5); a carbohydrate or sucrose present at a concentration of 70 mM to 75 mM, e.g., 73.3 mM; and a surfactant or polysorbate 20 present at a concentration of 0.01% to 0.015%, e.g., 0.013% (w/w).

In some embodiments, the formulation (e.g., drug substance formulation) comprises the combination according to the invention at a concentration of 33.3 mg/mL; a buffering agent that comprises histidine at a concentration of 6.7 mM and has a pH of 5.5; sucrose present at a concentration of 73.3 mM; and polysorbate 20 present at a concentration of 0.013% (w/w).

In some embodiments, the formulation is a lyophilized formulation. In certain embodiments, the lyophilized formulation is lyophilized from a drug substance formulation described herein. For example, 2 to 5 mL, e.g., 3 to 4 mL, e.g., 3.6 mL, of the drug substance formulation described herein can be filled per container (e.g., vial) and lyophilized.

In certain embodiments, the formulation is a reconstituted formulation. For example, a reconstituted formulation can be prepared by dissolving a lyophilized formulation in a diluent such that the drug substance is dispersed in the reconstituted formulation. In some embodiments, the lyophilized formulation is reconstituted with 0.5 mL to 2 mL, e.g., 1 mL, of water or buffer for injection. In certain embodiments, the lyophilized formulation is reconstituted with 1 mL of water for injection, e.g., at a clinical site.

In some embodiments, the formulation (e.g., reconstituted formulation) comprises the combination according to the invention comprising a PSMA therapeutic agent, such as radiolabeled Compound I described herein, and additional therapeutic agents, such as an Immuno-Oncology therapeutic agent, for example PD-1 inhibitor, a LAG-3 inhibitor, a TIM-3 inhibitor, a GITR agonist, a SERD, a CDK4/6 inhibitor, a CXCR2 inhibitor, a CSF-1/1R binding agent, a c-MET inhibitor, a TGF-β inhibitor, an A2aR antagonist, an IDO inhibitor, a MEK inhibitor, an IL-15/IL-15RA complex, an IL-1β inhibitor, or any combination thereof, and a buffering agent.

In some embodiments, the formulation (e.g., reconstituted formulation) comprises the combination according to the invention at a concentration of 20 mg/mL to 200 mg/mL, e.g., 50 mg/mL to 150 mg/mL, 80 mg/mL to 120 mg/mL, or 90 mg/mL to 110 mg/mL, e.g., 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL. In certain embodiments, the combination according to the invention is present at a concentration of 80 to 120 mg/mL, e.g., 100 mg/mL.

In some embodiments, the formulation (e.g., reconstituted formulation) comprises a buffering agent comprising histidine (e.g., a histidine buffer). In certain embodiments, the buffering agent (e.g., histidine buffer) is present at a concentration of 5 mM to 100 mM, e.g., 10 mM to 50 mM, 15 mM to 25 mM, e.g., 5 mM, 10 mM, 20 mM, 30 mM, 40 mM, 50 mM, 60 mM, 70 mM, 80 mM, 90 mM, or 100 mM. In some embodiments, the buffering agent (e.g., histidine buffer) is present at a concentration of 15 mM to 25 mM, e.g., 20 mM. In other embodiments, the buffering agent (e.g., a histidine buffer) has a pH of 4 to 7, e.g., 5 to 6, e.g., 5, 5.5 or 6. In some embodiments, the buffering agent (e.g., histidine buffer) has a pH of 5 to 6, e.g., 5.5. In certain embodiments, the buffering agent comprises histidine at a concentration of 15 mM to 25 mM (e.g., 20 mM) and has a pH of 5 to 6 (e.g., 5.5).

In some embodiments, the formulation (e.g., reconstituted formulation) comprises the combination according to the invention at a concentration of 80 to 120 mg/mL, e.g., 100 mg/mL; and a buffering agent that comprises histidine at a concentration of 6 mM to 7 mM (e.g., 6.7 mM) and has a pH of 5 to 6 (e.g., 5.5).

In some embodiments, the formulation (e.g., reconstituted formulation) further comprises a carbohydrate. In certain embodiments, the carbohydrate is sucrose. In some embodiments, the carbohydrate (e.g., sucrose) is present at a concentration of 100 mM to 500 mM, e.g., 150 mM to 400 mM, 175 mM to 300 mM, or 200 mM to 250 mM, e.g., 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, or 300 mM. In some embodiments, the formulation comprises a carbohydrate or sucrose present at a concentration of 200 mM to 250 mM, e.g., 220 mM.

In some embodiments, the formulation (e.g., reconstituted formulation) comprises the combination according to the invention present at a concentration of 80 to 120 mg/mL, e.g., 100 mg/mL; and a buffering agent that comprises histidine at a concentration of 6 mM to 7 mM (e.g., 6.7 mM) and has a pH of 5 to 6 (e.g., 5.5); and a carbohydrate or sucrose present at a concentration of 200 mM to 250 mM, e.g., 220 mM.

In some embodiments, the formulation (e.g., reconstituted formulation) further comprises a surfactant. In certain embodiments, the surfactant is polysorbate 20. In some embodiments, the surfactant or polysorbate 20 is present at a concentration of 0.01% to 0.1% (w/w), e.g., 0.02% to 0.08%, 0.025% to 0.06% or 0.03% to 0.05% (w/w), e.g., 0.01%, 0.025%, 0.03%, 0.04%, 0.05%, 0.06%, 0.07%, 0.08%, 0.09%, or 0.1% (w/w). In some embodiments, the formulation comprises a surfactant or polysorbate 20 present at a concentration of 0.03% to 0.05%, e.g., 0.04% (w/w).

In some embodiments, the formulation (e.g., reconstituted formulation) comprises the combination according to the invention at a concentration of 80 to 120 mg/mL, e.g., 100 mg/mL; and a buffering agent that comprises histidine at a concentration of 6 mM to 7 mM (e.g., 6.7 mM) and has a pH of 5 to 6 (e.g., 5.5); and a surfactant or polysorbate 20 present at a concentration of 0.03% to 0.05%, e.g., 0.04% (w/w).

In some embodiments, the formulation (e.g., reconstituted formulation) comprises the combination according to the invention at a concentration of 80 to 120 mg/mL, e.g., 100 mg/mL; and a buffering agent that comprises histidine at a concentration of 6 mM to 7 mM (e.g., 6.7 mM) and has a pH of 5 to 6 (e.g., 5.5); a carbohydrate or sucrose present at a concentration of 200 mM to 250 mM, e.g., 220 mM; and a surfactant or polysorbate 20 present at a concentration of 0.03% to 0.05%, e.g., 0.04% (w/w).

In some embodiments, the formulation (e.g., reconstituted formulation) comprises the combination according to the invention at a concentration of 100 mg/mL; and a buffering agent that comprises histidine at a concentration of 6.7 mM and has a pH of 5.5; sucrose present at a concentration of 220 mM; and polysorbate 20 present at a concentration of 0.04% (w/w).

In some embodiments, the formulation is reconstituted such that an extractable volume of at least 1 mL (e.g., at least 1.5 mL, 2 mL, 2.5 mL, or 3 mL) of the reconstituted formulation can be withdrawn from the container (e.g., vial) containing the reconstituted formulation. In certain embodiments, the formulation is reconstituted and/or extracted from the container (e.g., vial) at a clinical site. In certain embodiments, the formulation (e.g., reconstituted formulation) is injected to an infusion bag, e.g., within 1 hour (e.g., within 45 minutes, 30 minutes, or 15 minutes) before the infusion starts to the patient.

In certain embodiments, the formulation is a liquid formulation. In some embodiments, the liquid formulation is prepared by diluting a drug substance formulation described herein. For example, a drug substance formulation can be diluted, e.g., with 10 to 30 mg/mL (e.g., 25 mg/mL) of a solution comprising one or more excipients (e.g., concentrated excipients). In some embodiments, the solution comprises one, two, or all of histidine, sucrose, or polysorbate 20. In certain embodiments, the solution comprises the same excipient(s) as the drug substance formulation. Exemplary excipients include, but are not limited to, an amino acid (e.g., histidine), a carbohydrate (e.g., sucrose), or a surfactant (e.g., polysorbate 20). In certain embodiments, the liquid formulation is not a reconstituted lyophilized formulation. In other embodiments, the liquid formulation is a reconstituted lyophilized formulation. In some embodiments, the formulation is stored as a liquid. In other embodiments, the formulation is prepared as a liquid and then is dried, e.g., by lyophilization or spray-drying, prior to storage.

In some embodiments, the formulation (e.g., liquid formulation) the combination according to the invention comprising a PSMA therapeutic agent, such as radiolabeled Compound I described herein, and therapeutic agents, such as an Immuno-Oncology therapeutic agent previously described at a concentration of 5 mg/mL to 50 mg/mL, e.g., 10 mg/mL to 40 mg/mL, 15 mg/mL to 35 mg/mL, or 20 mg/mL to 30 mg/mL, e.g., 5 mg/mL, 10 mg/mL, 15 mg/mL, 20 mg/mL, 25 mg/mL, 30 mg/mL, 35 mg/mL, 40 mg/mL, 45 mg/mL, or 50 mg/mL. In certain embodiments, the PD-1 inhibitor (e.g., the anti-PD-1 antibody molecule) is present at a concentration of 20 to 30 mg/mL, e.g., 25 mg/mL.

In some embodiments, the formulation (e.g., liquid formulation) comprises a buffering agent comprising histidine (e.g., a histidine buffer). In certain embodiments, the buffering agent (e.g., histidine buffer) is present at a concentration of 5 mM to 100 mM, e.g., 10 mM to 50 mM, 15 mM to 25 mM, e.g., 5 mM, 10 mM, 20 mM, 30 mM, 40 mM, 50 mM, 60 mM, 70 mM, 80 mM, 90 mM, or 100 mM. In some embodiments, the buffering agent (e.g., histidine buffer) is present at a concentration of 15 mM to 25 mM, e.g., 20 mM. In other embodiments, the buffering agent (e.g., a histidine buffer) has a pH of 4 to 7, e.g., 5 to 6, e.g., 5, 5.5 or 6. In some embodiments, the buffering agent (e.g., histidine buffer) has a pH of 5 to 6, e.g., 5.5. In certain embodiments, the buffering agent comprises histidine at a concentration of 15 mM to 25 mM (e.g., 20 mM) and has a pH of 5 to 6 (e.g., 5.5).

In some embodiments, the formulation (e.g., liquid formulation) comprises the combination according to the invention at a concentration of 20 to 30 mg/mL, e.g., 25 mg/mL; and a buffering agent that comprises histidine at a concentration of 6 mM to 7 mM (e.g., 6.7 mM) and has a pH of 5 to 6 (e.g., 5.5).

In some embodiments, the formulation (e.g., liquid formulation) further comprises a carbohydrate. In certain embodiments, the carbohydrate is sucrose. In some embodiments, the carbohydrate (e.g., sucrose) is present at a concentration of 100 mM to 500 mM, e.g., 150 mM to 400 mM, 175 mM to 300 mM, or 200 mM to 250 mM, e.g., 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, or 300 mM. In some embodiments, the formulation comprises a carbohydrate or sucrose present at a concentration of 200 mM to 250 mM, e.g., 220 mM.

In some embodiments, the formulation (e.g., liquid formulation) comprises a PD-1 inhibitor (e.g., an anti-PD-1 antibody molecule) present at a concentration of 20 to 30 mg/mL, e.g., 25 mg/mL; and a buffering agent that comprises histidine at a concentration of 6 mM to 7 mM (e.g., 6.7 mM) and has a pH of 5 to 6 (e.g., 5.5); and a carbohydrate or sucrose present at a concentration of 200 mM to 250 mM, e.g., 220 mM.

In some embodiments, the formulation (e.g., liquid formulation) further comprises a surfactant. In certain embodiments, the surfactant is polysorbate 20. In some embodiments, the surfactant or polysorbate 20 is present at a concentration of 0.01% to 0.1% (w/w), e.g., 0.02% to 0.08%, 0.025% to 0.06% or 0.03% to 0.05% (w/w), e.g., 0.01%, 0.025%, 0.03%, 0.04%, 0.05%, 0.06%, 0.07%, 0.08%, 0.09%, or 0.1% (w/w). In some embodiments, the formulation comprises a surfactant or polysorbate 20 present at a concentration of 0.03% to 0.05%, e.g., 0.04% (w/w).

In some embodiments, the formulation (e.g., liquid formulation) comprises the combination according to the invention at a concentration of 20 to 30 mg/mL, e.g., 25 mg/mL; and a buffering agent that comprises histidine at a concentration of 6 mM to 7 mM (e.g., 6.7 mM) and has a pH of 5 to 6 (e.g., 5.5); and a surfactant or polysorbate 20 present at a concentration of 0.03% to 0.05%, e.g., 0.04% (w/w).

In some embodiments, the formulation (e.g., liquid d formulation) comprises the combination according to the invention at a concentration of 20 to 30 mg/mL, e.g., 25 mg/mL; and a buffering agent that comprises histidine at a concentration of 6 mM to 7 mM (e.g., 6.7 mM) and has a pH of 5 to 6 (e.g., 5.5); a carbohydrate or sucrose present at a concentration of 200 mM to 250 mM, e.g., 220 mM; and a surfactant or polysorbate 20 present at a concentration of 0.03% to 0.05%, e.g., 0.04% (w/w).

In some embodiments, the formulation (e.g., liquid formulation) comprises the combination according to the invention at a concentration of 25 mg/mL; and a buffering agent that comprises histidine at a concentration of 6.7 mM and has a pH of 5.5; sucrose present at a concentration of 220 mM; and polysorbate 20 present at a concentration of 0.04% (w/w).

In certain embodiments, 1 mL to 10 mL (e.g., 2 mL to 8 mL, 3 mL to 7 mL, or 4 mL to 5 mL, e.g., 3 mL, 4 mL, 4.3 mL, 4.5 mL, 5 mL, or 6 mL) of the liquid formulation is filled per container (e.g., vial). In other embodiments, the liquid formulation is filled into a container (e.g., vial) such that an extractable volume of at least 2 mL (e.g., at least 3 mL, at least 4 mL, or at least 5 mL) of the liquid formulation can be withdrawn per container (e.g., vial). In certain embodiments, the liquid formulation is diluted from the drug substance formulation and/or extracted from the container (e.g., vial) at a clinical site. In certain embodiments, the formulation (e.g., liquid formulation) is injected to an infusion bag, e.g., within 1 hour (e.g., within 45 minutes, 30 minutes, or 15 minutes) before the infusion starts to the patient.

A formulation described herein can be stored in a container. The container used for any of the formulations described herein can include, e.g., a vial, and optionally, a stopper, a cap, or both. In certain embodiments, the vial is a glass vial, e.g., a 6R white glass vial. In other embodiments, the stopper is a rubber stopper, e.g., a grey rubber stopper. In other embodiments, the cap is a flip-off cap, e.g., an aluminum flip-off cap. In some embodiments, the container comprises a 6R white glass vial, a grey rubber stopper, and an aluminum flip-off cap. In some embodiments, the container (e.g., vial) is a single-use container. In certain embodiments, 50 mg to 150 mg, e.g., 80 mg to 120 mg, 90 mg to 110 mg, 100 mg to 120 mg, 100 mg to 110 mg, 110 mg to 120 mg, or 110 mg to 130 mg, of the drug substance, is present in the container (e.g., vial).

Other exemplary buffering agents that can be used in the formulations described herein include, but are not limited to, an arginine buffer, a citrate buffer, or a phosphate buffer. Other exemplary carbohydrates that can be used in the formulation described herein include, but are not limited to, trehalose, mannitol, sorbitol, or a combination thereof. The formulations described herein may also contain a tonicity agent, e.g., sodium chloride, and/or a stabilizing agent, e.g., an amino acid (e.g., glycine, arginine, methionine, or a combination thereof).

The combination according to the invention, inhibitors, antagonist or binding agents, can be administered by a variety of methods known in the art, although for many therapeutic applications, a suitable route/mode of administration is intravenous injection or infusion. For example, the PSMA therapeutic agent, such as radiolabeled Compound I, or other therapeutic agents can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m².

In certain embodiments, the composition according to the invention and the other therapeutic agents can be administered by intravenous infusion at a rate of less than 10 mg/min, or less than or equal to 5 mg/min to reach a dose of about 1 to 100 mg/m², about 5 to 50 mg/m², about 7 to 25 mg/m², or about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. In various embodiments, biodegradable, biocompatible polymers can be used, such as ethylene vinyl acetate, polyanhydrides, polyglycolic acid, collagen, polyorthoesters, and polylactic acid. Many methods for the preparation of such formulations are generally known to those skilled in the art. See, e.g., Sustained and Controlled Release Drug Delivery Systems, J. R. Robinson, ed., Marcel Dekker, Inc., New York, 1978.

In certain embodiments, combination according to the invention can be orally administered, for example, with an inert diluent or an assimilable edible carrier. In another embodiment, any of the therapeutic agents described herein (and other ingredients, if desired) may also be enclosed in a hard or soft shell gelatin capsule, compressed into tablets, or incorporated directly into the subject's diet. For oral therapeutic administration, the therapeutic agents may be incorporated with excipients and used in the form of ingestible tablets, buccal tablets, troches, capsules, elixirs, suspensions, syrups, wafers, and the like. In one embodiment, to administer a therapeutic agent of the disclosure by other than parenteral administration, it may be necessary to coat the therapeutic agent with, or co-administer the therapeutic agent with, a material to prevent its inactivation. In another aspect, therapeutic compositions can also be administered with medical devices known in the art.

Dosage regimens can be adjusted to provide the optimum desired response (e.g., a therapeutic response). For example, a single bolus may be administered, several divided doses may be administered over time or the dose may be proportionally reduced or increased as indicated by the exigencies of the therapeutic situation. In one embodiment, parenteral compositions can be formulated in dosage unit form for ease of administration and uniformity of dosage. Dosage unit form as used herein refers to physically discrete units suited as unitary dosages for the subjects to be treated; each unit may contain a predetermined quantity of active compound calculated to produce the desired therapeutic effect in association with the required pharmaceutical carrier. In various aspects, the specification for the dosage unit forms of the invention are dictated by and directly dependent on (a) the unique characteristics of the active compound and the particular therapeutic effect to be achieved, and (b) the limitations inherent in the art of compounding such an active compound for the treatment of the subject.

An exemplary, non-limiting range for a therapeutically or prophylactically effective amount of a therapeutic agent, such as PSMA therapeutic agent as described above, is 0.1-30 mg/kg, or 1-25 mg/kg. Dosages and therapeutic regimens of can be determined by a skilled artisan. In certain embodiments, the therapeutic agent, such as PSMA therapeutic agent as described above, is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In one embodiment, the therapeutic agent is administered at a dose from about 10 to 20 mg/kg every other week.

As another example, a non-limiting range for a therapeutically or prophylactically effective amount of therapeutic agent described herein is 200-500 mg, or 300-400 mg/kg. In certain embodiments, the therapeutic agent, such as PSMA therapeutic agent as described above, is administered by injection (e.g., subcutaneously or intravenously) at a dose (e.g., a flat dose) of about 200 mg to 500 mg, e.g., about 250 mg to 450 mg, about 300 mg to 400 mg, about 250 mg to 350 mg, about 350 mg to 450 mg, or about 300 mg or about 400 mg. The dosing schedule (e.g., flat dosing schedule) can vary from e.g., once a week to once every 2, 3, 4, 5, or 6 weeks.

In one embodiment the therapeutic agent is administered at a dose from about 300 mg to 400 mg once every three or once every four weeks.

In one embodiment, the therapeutic agent is administered at a dose from about 300 mg once every three weeks.

In one embodiment, the combination according to the invention is administered at a dose from about 400 mg once every four weeks. In one embodiment, the combination according to the invention is administered at a dose from about 300 mg once every four weeks. In one embodiment, the combination according to the invention is administered at a dose from about 400 mg once every three weeks. While not wishing to be bound by theory, in some embodiments, flat or fixed dosing can be beneficial to patients, for example, to save drug supply and to reduce pharmacy errors.

In some embodiments, the clearance (CL) of the combination according to the invention is from about 6 to 16 mL/h, e.g., about 7 to 15 mL/h, about 8 to 14 mL/h, about 9 to 12 mL/h, or about 10 to 11 mL/h, e.g., about 8.9 mL/h, 10.9 mL/h, or 13.2 mL/h.

In some embodiments, the exponent of weight on CL of the combination according to the invention is from about 0.4 to 0.7, about 0.5 to 0.6, or 0.7 or less, e.g., 0.6 or less, or about 0.54.

In some embodiments, the volume of distribution at steady state (Vss) of the combination according to the invention is from about 5 to 10 V, e.g., about 6 to 9 V, about 7 to 8 V, or about 6.5 to 7.5 V, e.g., about 7.2 V.

In some embodiments, the half-life of the combination according to the invention is from about 10 to 30 days, e.g., about 15 to 25 days, about 17 to 22 days, about 19 to 24 days, or about 18 to 22 days, e.g., about 20 days.

In some embodiments, the Cmin (e.g., for a 80 kg patient) of the combination according to the invention is at least about 0.4 μg/mL, e.g., at least about 3.6 μg/mL, e.g., from about 20 to 50 μg/mL, e.g., about 22 to 42 μg/mL, about 26 to 47 μg/mL, about 22 to 26 μg/mL, about 42 to 47 μg/mL, about 25 to 35 μg/mL, about 32 to 38 μg/mL, e.g., about 31 μg/mL or about 35 μg/mL.

In one embodiment, the Cmin is determined in a patient receiving the combination according to the invention at a dose of about 400 mg once every four weeks.

In another embodiment, the Cmin is determined in a patient receiving the combination according to the invention at a dose of about 300 mg once every three weeks.

In certain embodiments, the Cmin is at least about 50-fold higher, e.g., at least about 60-fold, 65-fold, 70-fold, 75-fold, 80-fold, 85-fold, 90-fold, 95-fold, or 100-fold, e.g., at least about 77-fold, higher than the EC50 of the therapeutic agent, e.g., as determined based on IL-2 change in an SEB ex-vivo assay. In other embodiments, the Cmin is at least 5-fold higher, e.g., at least 6-fold, 7-fold, 8-fold, 9-fold, or 10-fold, e.g., at least about 8.6-fold, higher than the EC90 of the therapeutic agent, e.g., as determined based on IL-2 change in an SEB ex-vivo assay.

The PSMA therapeutic agent, such as radiolabeled Compound I, or the additional therapeutic agent can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m².

In embodiments, the infusion rate of about 110 to 130 mg/m² achieves a level of about 3 mg/kg.

In other embodiments, the combination according to the invention can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or, about 10 mg/m².

In some embodiments, the PSMA therapeutic agent, such as radiolabeled Compound I, or another therapeutic agent is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include a “therapeutically effective amount” or a “prophylactically effective amount” of a combination according to the invention.

A “therapeutically effective amount” refers to an amount effective, at dosages and for periods of time necessary, to achieve the desired therapeutic result. A therapeutically effective amount of the combination according to the invention may vary according to factors such as the disease state, age, sex, and weight of the individual, and the ability of the PSMA therapeutic agent, such as radiolabeled Compound I, and additional therapeutic agent, such as the Immuno-Oncology therapeutic agent, to elicit a desired response in the individual. A therapeutically effective amount is also one in which any toxic or detrimental effects of the PSMA therapeutic agent, such as radiolabeled Compound I, or another therapeutic agent is outweighed by the therapeutically beneficial effects.

A “therapeutically effective dosage” can inhibit a measurable parameter, e.g., tumor growth rate by at least about 20%, by at least about 40%, by at least about 60%, or by at least about 80% relative to untreated subjects. The ability of the combination according to the invention to inhibit a measurable parameter, e.g., cancer, can be evaluated in an animal model system predictive of efficacy in human tumors. Alternatively, this property of a composition can be evaluated by examining the ability of the combination according to the invention to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

### Kits

A combination of therapeutic agents disclosed herein can be provided in a kit.

In one embodiment, the combination according to the invention which comprises a PSMA therapeutic agent, such as radiolabeled Compound I and additional therapeutic agent such as Immuno-Oncology therapeutic agent described herein is provided in a vial or a container.

As appropriate, the PSMA therapeutic agents and the additional therapeutic agent, such as Immuno-Oncology therapeutic agent, can be in liquid or dried (e.g., lyophilized) form.

In one aspect, the kits can comprise one or more (e.g., one, two, three, four, five, or all) of the PSMA therapeutic agent, such as radiolabeled Compound I and additional therapeutic agent such as Immuno-Oncology therapeutic agent disclosed herein.

In some embodiments, the kit further contains a pharmaceutically acceptable diluent.

In various embodiments, the PSMA therapeutic agent, such as radiolabeled Compound I and additional therapeutic agent such as Immuno-Oncology therapeutic agent can be provided in the kit in the same or separate formulations (e.g., as mixtures or in separate containers).

The kits can contain aliquots of said PSMA therapeutic agent and the additional therapeutic agent, such as Immuno-Oncology therapeutic agents, that provide for one or more doses. If aliquots for multiple administrations are provided, the doses can be uniform or varied. For example, varied dosing regimens can be escalating or decreasing, as appropriate. In one aspect, the dosages of the therapeutic agents in the combination can be independently uniform or varying. In additional embodiments, the kit can include one or more other elements including: instructions for use; other reagents, e.g., a label, or an agent useful for chelating, or otherwise coupling, a therapeutic agent to a label a therapeutic agent, or a radioprotective composition; devices or other materials for preparing the therapeutic agents for administration; pharmaceutically acceptable carriers; and devices or other materials for administration to a subject.

### INCORPORATION BY REFERENCE

All publications, patents, and Accession numbers mentioned herein are hereby incorporated herein by reference in their entirety as if each individual publication or patent was specifically and individually indicated to be incorporated herein by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

### EXAMPLES

Radiolabeled Compound I can be prepared according to the methods described in WO2015055318, incorporated herein by reference for the preparation of radiolabeled Compound I. The preparation of radiolabeled Compound I is briefly described below.

### Example 1: Synthesis of Compound I

Compounds I can be synthesized using the methods disclosed in WO2017/165473.

In particular, Compound I can be synthesized as disclosed in scheme 1. The p-bromobenzyl group modified of Glu-Lys urea (2) can be prepared by reductive alkylation of Glu-Lys urea (1) with p-bromobenzaldehyde in presence of sodium cyanoborohydride in methanol according to the method described in Tykvart, et al. (2015) Journal of medicinal chemistry 58, 4357-63. Then, an aliphatic linker, Boc-6-aminohexanoic acid can be coupled to the same ε-Lys amine of (2), for example, using a base (like N,N-diisopropylethylamine) and a coupling agent (like N,N,N′,N′-Tetramethyl-O—(N-succinimidyl)uronium tetrafluoroborate or 1-[Bis(dimethylamino)methylene]-1H-1,2,3-triazolo[4,5-b]pyridinium 3-oxid hexafluoro-phosphate), to yield compound (3). Compound (3) can then be deprotected to yield compound (4), for example using an acid like trifluoroacetic acid. Finally, conjugation with commercially available DOT A-NHS ester can be performed to yield Compound I.

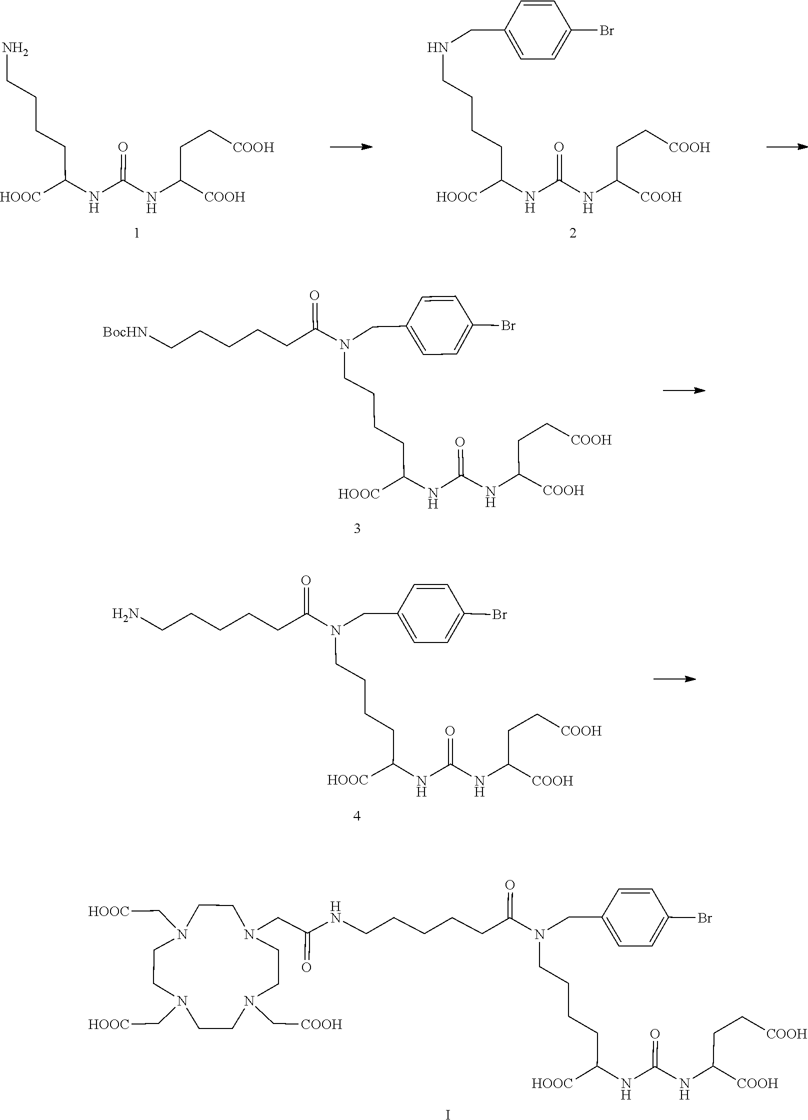

### Example 2: Radiolabeling of Compound I

Compound I can be radiolabeled using methods which are commonly used in the field of radiolabeling. In particular, Compound I can also be radiolabeled with ¹⁷⁷Lu, to form the ¹⁷⁷Lu radiolabeled Compound I, using the method described in WO2017/165473.

1. A combination comprising a compound of Formula I (Compound I) 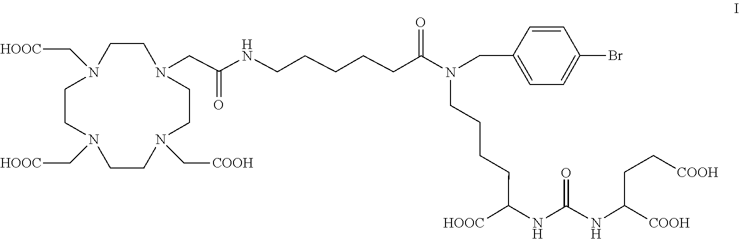 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]
wherein Compound I is radiolabeled and one or more immuno-oncology (I-O) therapeutic agent(s) for use in treating a PSMA expressing cancer in a subject, wherein said I-O therapeutic agent(s) is (are) selected from LAG-3 inhibitors, TIM-3 inhibitors, GITR angonists, TGF-β inhibitors, IL15/IL-15RA complexes, PD-1 inhibitors, PD-L1 inhibitors, and CTLA-4 inhibitors, wherein said PD-1 inhibitors are selected from Spartalizumab, Pembrolizumab, Pidilizumab, Durvalomab, Atezolizumab, Avelumab, Nivolumab, MK-3475, MPDL3280A, MEDI4736, ipilimumab, tremelimumab, MEDI0680, REGN2810, TSR-042, PF-06801591, BGB-A317, BGB-108, INCSHR1210, and AMP-224.

2. A method of treating a PSMA expressing cancer in a subject,
comprising administering to the subject a combination of a compound of Formula I (Compound I) 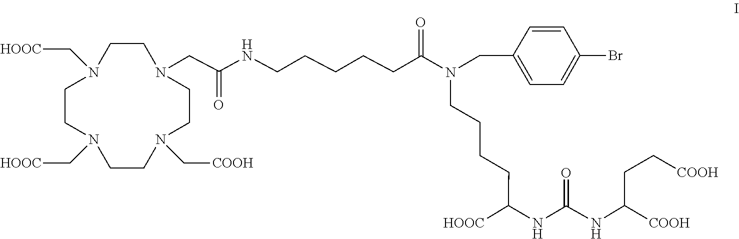 [SMILES: O=C(O)CCC(NC(=O)NC(CCCCN(Cc1ccc(Br)cc1)C(=O)CCCCCNC(=O)CN1CCN(CC(=O)O)CCN(CC(=O)O)CCN(CC(=O)O)CC1)C(=O)O)C(=O)O]
wherein Compound I is radiolabeled and one or more immuno-oncology (I-O) therapeutic agent(s), wherein said I-O therapeutic agent(s) is(are) selected from an LAG-3 inhibitor, a TIM-3 inhibitor, a GITR angonists, a TGF-β inhibitor, an IL15/IL-15RA complex, and a PD-1 inhibitor, wherein said PD-1 inhibitor is selected from Spartalizumab, Pembrolizumab, Pidilizumab, Durvalomab, Atezolizumab, Avelumab, Nivolumab, MK-3475, MPDL3280A, MEDI4736, ipilimumab, tremelimumab, MEDI0680, REGN2810, TSR-042, PF-06801591, BGB-A317, BGB-108, INCSHR1210, and AMP-224.

3. The combination of claim 1, wherein the radiolabeled Compound I and the I-O therapeutic agent(s) are in separate compositions and are administered to the subject separately.

4. The combination of claim 1, wherein the LAG-3 inhibitor is chosen from LAG525, BMS-986016, or TSR-033, wherein the TIM-3 inhibitor is MBG453 or TSR-022, wherein the GITR agonist is chosen from GWN323, BMS-986156, MK-4166, MK-1248, TRX518, INCAGN1876, AMG 228, or INBRX-110, wherein the TGF-β inhibitor is XOMA 089 or fresolimumab, wherein the IL-15/IL-15RA complex is chosen from NIZ985, ATL-803 or CYP0150 and wherein the IL-15/IL-15RA complex is chosen from NIZ985, ATL-803 or CYP0150.

5. The combination of claim 1, comprising one or more further anti-cancer agent(s) selected from octreotide, lanreotide, vaproreotide, pasireotide, satoreotide, everolimus, temozolomide, telotristat, sunitinib, sulfatinib, ribociclib, entinostat, and pazopanib.

6. The combination of claim 1, wherein the PSMA expressing cancer is a prostate cancer.

7. The combination of claim 1, wherein the PSMA-expressing cancer is selected from thyroid cancer, renal clear cell carcinoma, transitional cell carcinoma of the bladder, colonic adenocarcinoma, neuroendocrine carcinoma, glioblastoma multiforme, malignant melanoma, pancreatic duct carcinoma, non-small cell lung carcinoma, soft tissue sarcoma, and breast carcinoma.

8. The combination of claim 1, wherein Compound I is bound to a radionuclide selected from ¹⁷⁷Lu and ²²⁵Ac.

9. The combination claim 8 wherein both the Compound I bound to ¹⁷⁷Lu and the Compound I bound to ²²⁵Ac are administered to the subject.

10. The combination of claim 1, wherein a PD-1 inhibitor is administered, and the PD-1 inhibitor is not Pembrolizumab.

11. The combination of claim 9, wherein the amount of the Compound I bound to ¹⁷⁷Lu that is administered is from about 2 GBq to about 13 GBq.

12. The combination of claim 9, wherein the amount of the Compound I bound to ²²⁵Ac that is administered is from about 1 MBq to about 6 MBq.

13. The combination of claim 1, wherein the I-O therapeutic agent is nivolumab, ipilimumab or tremelimumab.

14. The method of claim 2, wherein the radiolabeled Compound I and the I-O therapeutic agent(s) are in separate compositions and are administered to the subject separately.

15. The method of claim 2, wherein the LAG-3 inhibitor is chosen from LAG525, BMS-986016, or TSR-033, wherein the TIM-3 inhibitor is MBG453 or TSR-022, wherein the GITR agonist is chosen from GWN323, BMS-986156, MK-4166, MK-1248, TRX518, INCAGN1876, AMG 228, or INBRX-110, wherein the TGF-β inhibitor is XOMA 089 or fresolimumab, wherein the IL-15/IL-15RA complex is chosen from NIZ985, ATL-803 or CYP0150 and wherein the IL-15/IL-15RA complex is chosen from NIZ985, ATL-803 or CYP0150.

16. The method of claim 2, comprising administering one or more further anti-cancer agent(s) selected from octreotide, Ianreotide, vaproreotide, pasireotide, satoreotide, everolimus, temozolomide, telotristat, sunitinib, sulfatinib, ribociclib, entinostat, and pazopanib.

17. The method of claim 2, wherein the PSMA expressing cancer is a prostate cancer.

18. The method of claim 2, wherein the PSMA-expressing cancer is selected from thyroid cancer, renal clear cell carcinoma, transitional cell carcinoma of the bladder, colonic adenocarcinoma, neuroendocrine carcinoma, glioblastoma multiforme, malignant melanoma, pancreatic duct carcinoma, non-small cell lung carcinoma, soft tissue sarcoma, and breast carcinoma.

19. The method of claim 2, wherein Compound I is bound to a radionuclide selected from ¹⁷⁷Lu and ²²⁵Ac.

20. The method of claim 19 wherein both the Compound I bound to ¹⁷⁷Lu and the Compound I bound to ²²⁵Ac are administered to the subject.

21. The method of claim 2, wherein a PD-1 inhibitor is administered, and the PD-1 inhibitor is not Pembrolizumab.

22. The method of claim 20, wherein the amount of the Compound I bound to ¹⁷⁷Lu that is administered is from about 2 GBq to about 13 GBq.

23. The method of claim 20, wherein the amount of the Compound I bound to ²²⁵Ac that is administered is from about 1 MBq to about 6 MBq.

24. The method of claim 2, wherein the I-O therapeutic agent is nivolumab, ipilimumab or tremelimumab.


SEQUENCE LISTING (171 sequences)

SEQ ID NO: 1 - protein, 5 aa, Artificial Sequence
  Antibody fragment
TYWMH

SEQ ID NO: 2 - protein, 17 aa, Artificial Sequence
  Antibody fragment
NIYPGTGGSNFDEKFKN

SEQ ID NO: 3 - protein, 8 aa, Artificial Sequence
  Antibody fragment
WTTGTGAY

SEQ ID NO: 4 - protein, 7 aa, Artificial Sequence
  Antibody fragment
GYTFTTY

SEQ ID NO: 5 - protein, 6 aa, Artificial Sequence
  Antibody fragment
YPGTGG

SEQ ID NO: 6 - protein, 8 aa, Artificial Sequence
  Antibody fragment
WTTGTGAY

SEQ ID NO: 7 - DNA, 351 bp, Artificial Sequence
  antibody sequence
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 8 - protein, 443 aa, Artificial Sequence
  Antibody fragment
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 9 - DNA, 1329 bp, Artificial Sequence
  Antibody fragment
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 10 - protein, 17 aa, Artificial Sequence
  Antibody fragment
KSSQSLLDSGNQKNFLT

SEQ ID NO: 11 - protein, 7 aa, Artificial Sequence
  Antibody fragment
WASTRES

SEQ ID NO: 12 - protein, 9 aa, Artificial Sequence
  Antibody fragment
QNDYSYPYT

SEQ ID NO: 13 - protein, 13 aa, Artificial Sequence
  Antibody fragment
SQSLLDSGNQKNF

SEQ ID NO: 14 - protein, 3 aa, Artificial Sequence
  Antibody fragment
WAS

SEQ ID NO: 15 - protein, 6 aa, Artificial Sequence
  Antibody fragment
DYSYPY

SEQ ID NO: 16 - protein, 113 aa, Artificial Sequence
  Antibody fragment
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 17 - DNA, 339 bp, Artificial Sequence
  Antibody fragment
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 18 - protein, 220 aa, Artificial Sequence
  Antibody fragment
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 660 bp, Artificial Sequence
  Antibody fragment
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 20 - protein, 113 aa, Artificial Sequence
  Antibody fragment
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 21 - DNA, 339 bp, Artificial Sequence
  Antibody fragment
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 22 - protein, 220 aa, Artificial Sequence
  Antibody fragment
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 660 bp, Artificial Sequence
  Antibody fragment
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 24 - DNA, 15 bp, Artificial Sequence
  Antibody fragment
acctactggatgcac

SEQ ID NO: 25 - DNA, 51 bp, Artificial Sequence
  Antibody fragment
aatatctaccccggcaccggcggctctaacttcgacgagaagtttaagaat

SEQ ID NO: 26 - DNA, 24 bp, Artificial Sequence
  Antibody fragment
tggactaccggcacaggcgcctac

SEQ ID NO: 27 - DNA, 21 bp, Artificial Sequence
  Antibody fragment
ggctacaccttcactacctac

SEQ ID NO: 28 - DNA, 18 bp, Artificial Sequence
  Antibody fragment
taccccggcaccggcggc

SEQ ID NO: 29 - DNA, 51 bp, Artificial Sequence
  Antibody fragment
aaatctagtcagtcactgctggatagcggtaatcagaagaacttcctgacc

SEQ ID NO: 30 - DNA, 21 bp, Artificial Sequence
  Antibody fragment
tgggcctctactagagaatca

SEQ ID NO: 31 - DNA, 27 bp, Artificial Sequence
  Antibody fragment
cagaacgactatagctacccctacacc

SEQ ID NO: 32 - DNA, 39 bp, Artificial Sequence
  Antibody fragment
agtcagtcactgctggatagcggtaatcagaagaacttc

SEQ ID NO: 33 - DNA, 9 bp, Artificial Sequence
  Antibody fragment
tgggcctct

SEQ ID NO: 34 - DNA, 18 bp, Artificial Sequence
  Antibody fragment
gactatagctacccctac

SEQ ID NO: 35 - protein, 447 aa, Artificial Sequence
  Antibody fragment
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 218 aa, Artificial Sequence
  Antibody fragment
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 447 aa, Artificial Sequence
  Antibody fragment
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 213 aa, Artificial Sequence
  Antibody fragment
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 10 aa, Artificial Sequence
  Antibody fragment
GYTFTTYWMH

SEQ ID NO: 40 - protein, 5 aa, Artificial Sequence
  Antibody fragment
NYGMN

SEQ ID NO: 41 - protein, 17 aa, Artificial Sequence
  Antibody fragment
WINTDTGEPTYADDFKG

SEQ ID NO: 42 - protein, 16 aa, Artificial Sequence
  Antibody fragment
NPPYYYGTNNAEAMDY

SEQ ID NO: 43 - protein, 7 aa, Artificial Sequence
  Antibody fragment
GFTLTNY

SEQ ID NO: 44 - protein, 6 aa, Artificial Sequence
  Antibody fragment
NTDTGE

SEQ ID NO: 45 - protein, 125 aa, Artificial Sequence
  Antibody fragment
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSS

SEQ ID NO: 46 - DNA, 375 bp, Artificial Sequence
  Antibody fragment
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagc

SEQ ID NO: 47 - DNA, 375 bp, Artificial Sequence
  Antibody fragment
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 48 - protein, 451 aa, Artificial Sequence
  Antibody fragment
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 49 - protein, 11 aa, Artificial Sequence
  Antibody fragment
SSSQDISNYLN

SEQ ID NO: 50 - protein, 7 aa, Artificial Sequence
  Antibody fragment
YTSTLHL

SEQ ID NO: 51 - protein, 9 aa, Artificial Sequence
  Antibody fragment
QQYYNLPWT

SEQ ID NO: 52 - protein, 7 aa, Artificial Sequence
  Antibody fragment
SQDISNY

SEQ ID NO: 53 - protein, 3 aa, Artificial Sequence
  Antibody fragment
YTS

SEQ ID NO: 54 - protein, 6 aa, Artificial Sequence
  Antibody fragment
YYNLPW

SEQ ID NO: 55 - DNA, 1353 bp, Artificial Sequence
  Antibody fragment
caagtgcagctggtgcagtcgggagccgaagtgaagaagcctggagcctcggtgaaggtgtcgtgcaaggcatccggattcaccctcaccaattacgggatgaactgggtcagacaggcccggggtcaacggctggagtggatcggatggattaacaccgacaccggggagcctacctacgcggacgatttcaagggacggttcgtgttctccctcgacacctccgtgtccaccgcctacctccaaatctcctcactgaaagcggaggacaccgccgtgtactattgcgcgaggaacccgccctactactacggaaccaacaacgccgaagccatggactactggggccagggcaccactgtgactgtgtccagcgcgtccactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 56 - DNA, 1353 bp, Artificial Sequence
  Antibody fragment
caggtgcagctggtgcagtctggcgccgaagtgaagaaacctggcgcctccgtgaaggtgtcctgcaaggcctctggcttcaccctgaccaactacggcatgaactgggtgcgacaggccaggggccagcggctggaatggatcggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 57 - protein, 107 aa, Artificial Sequence
  Antibody fragment
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIK

SEQ ID NO: 58 - DNA, 321 bp, Artificial Sequence
  Antibody fragment
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaag

SEQ ID NO: 59 - DNA, 321 bp, Artificial Sequence
  Antibody fragment
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaag

SEQ ID NO: 60 - protein, 214 aa, Artificial Sequence
  Antibody fragment
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - DNA, 642 bp, Artificial Sequence
  Antibody fragment
gatattcagatgactcagtcacctagtagcctgagcgctagtgtgggcgatagagtgactatcacctgtagctctagtcaggatatctctaactacctgaactggtatctgcagaagcccggtcaatcacctcagctgctgatctactacactagcaccctgcacctgggcgtgccctctaggtttagcggtagcggtagtggcaccgagttcaccctgactatctctagcctgcagcccgacgacttcgctacctactactgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 62 - DNA, 642 bp, Artificial Sequence
  Antibody fragment
gacatccagatgacccagtccccctccagcctgtctgcttccgtgggcgacagagtgaccatcacctgttcctccagccaggacatctccaactacctgaactggtatctgcagaagcccggccagtcccctcagctgctgatctactacacctccaccctgcacctgggcgtgccctccagattttccggctctggctctggcaccgagtttaccctgaccatcagctccctgcagcccgacgacttcgccacctactactgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 63 - protein, 125 aa, Artificial Sequence
  Antibody fragment
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSS

SEQ ID NO: 64 - DNA, 375 bp, Artificial Sequence
  Antibody fragment
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagc

SEQ ID NO: 65 - DNA, 375 bp, Artificial Sequence
  Antibody fragment
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctct

SEQ ID NO: 66 - protein, 451 aa, Artificial Sequence
  Antibody fragment
QVQLVQSGAEVKKPGASVKVSCKASGFTLTNYGMNWVRQAPGQGLEWMGWINTDTGEPTYADDFKGRFVFSLDTSVSTAYLQISSLKAEDTAVYYCARNPPYYYGTNNAEAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 67 - DNA, 1353 bp, Artificial Sequence
  Antibody fragment
caggtgcagctggtgcagtcaggcgccgaagtgaagaaacccggcgctagtgtgaaagtcagctgtaaagctagtggcttcaccctgactaactacgggatgaactgggtccgccaggccccaggtcaaggcctcgagtggatgggctggattaacaccgacaccggcgagcctacctacgccgacgactttaagggcagattcgtgtttagcctggacactagtgtgtctaccgcctacctgcagatctctagcctgaaggccgaggacaccgccgtctactactgcgctagaaaccccccctactactacggcactaacaacgccgaggctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 68 - DNA, 1353 bp, Artificial Sequence
  Antibody fragment
caggtgcagctggtgcagtctggcgccgaagtgaagaaacctggcgcctccgtgaaggtgtcctgcaaggcctctggcttcaccctgaccaactacggcatgaactgggtgcgacaggcccctggacagggcctggaatggatgggctggatcaacaccgacaccggcgagcctacctacgccgacgacttcaagggcagattcgtgttctccctggacacctccgtgtccaccgcctacctgcagatctccagcctgaaggccgaggataccgccgtgtactactgcgcccggaaccccccttactactacggcaccaacaacgccgaggccatggactattggggccagggcaccaccgtgaccgtgtcctctgcttctaccaaggggcccagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggc

SEQ ID NO: 69 - protein, 107 aa, Artificial Sequence
  Antibody fragment
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIK

SEQ ID NO: 70 - DNA, 321 bp, Artificial Sequence
  Antibody fragment
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaag

SEQ ID NO: 71 - DNA, 321 bp, Artificial Sequence
  Antibody fragment
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaag

SEQ ID NO: 72 - protein, 214 aa, Artificial Sequence
  Antibody fragment
DIQMTQSPSSLSASVGDRVTITCSSSQDISNYLNWYQQKPGKAPKLLIYYTSTLHLGIPPRFSGSGYGTDFTLTINNIESEDAAYYFCQQYYNLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - DNA, 642 bp, Artificial Sequence
  Antibody fragment
gatattcagatgactcagtcacctagtagcctgagcgctagtgtgggcgatagagtgactatcacctgtagctctagtcaggatatctctaactacctgaactggtatcagcagaagcccggtaaagcccctaagctgctgatctactacactagcaccctgcacctgggaatcccccctaggtttagcggtagcggctacggcaccgacttcaccctgactattaacaatatcgagtcagaggacgccgcctactacttctgtcagcagtactataacctgccctggaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 74 - DNA, 642 bp, Artificial Sequence
  Antibody fragment
gacatccagatgacccagtccccctccagcctgtctgcttccgtgggcgacagagtgaccatcacctgttcctccagccaggacatctccaactacctgaactggtatcagcagaagcccggcaaggcccccaagctgctgatctactacacctccaccctgcacctgggcatcccccctagattctccggctctggctacggcaccgacttcaccctgaccatcaacaacatcgagtccgaggacgccgcctactacttctgccagcagtactacaacctgccctggaccttcggccagggcaccaaggtggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgc

SEQ ID NO: 75 - DNA, 15 bp, Artificial Sequence
  Antibody fragment
aattacgggatgaac

SEQ ID NO: 76 - DNA, 15 bp, Artificial Sequence
  Antibody fragment
aactacggcatgaac

SEQ ID NO: 77 - DNA, 51 bp, Artificial Sequence
  Antibody fragment
tggattaacaccgacaccggggagcctacctacgcggacgatttcaaggga

SEQ ID NO: 78 - DNA, 51 bp, Artificial Sequence
  Antibody fragment
tggatcaacaccgacaccggcgagcctacctacgccgacgacttcaagggc

SEQ ID NO: 79 - DNA, 48 bp, Artificial Sequence
  Antibody fragment
aacccgccctactactacggaaccaacaacgccgaagccatggactac

SEQ ID NO: 80 - DNA, 48 bp, Artificial Sequence
  Antibody fragment
aaccccccttactactacggcaccaacaacgccgaggccatggactat

SEQ ID NO: 81 - DNA, 21 bp, Artificial Sequence
  Antibody fragment
ggattcaccctcaccaattac

SEQ ID NO: 82 - DNA, 21 bp, Artificial Sequence
  Antibody fragment
ggcttcaccctgaccaactac

SEQ ID NO: 83 - DNA, 18 bp, Artificial Sequence
  Antibody fragment
aacaccgacaccggggag

SEQ ID NO: 84 - DNA, 18 bp, Artificial Sequence
  Antibody fragment
aacaccgacaccggcgag

SEQ ID NO: 85 - DNA, 33 bp, Artificial Sequence
  Antibody fragment
agctctagtcaggatatctctaactacctgaac

SEQ ID NO: 86 - DNA, 33 bp, Artificial Sequence
  Antibody fragment
tcctccagccaggacatctccaactacctgaac

SEQ ID NO: 87 - DNA, 21 bp, Artificial Sequence
  Antibody fragment
tacactagcaccctgcacctg

SEQ ID NO: 88 - DNA, 21 bp, Artificial Sequence
  Antibody fragment
tacacctccaccctgcacctg

SEQ ID NO: 89 - DNA, 27 bp, Artificial Sequence
  Antibody fragment
cagcagtactataacctgccctggacc

SEQ ID NO: 90 - DNA, 27 bp, Artificial Sequence
  Antibody fragment
cagcagtactacaacctgccctggacc

SEQ ID NO: 91 - DNA, 21 bp, Artificial Sequence
  Antibody fragment
agtcaggatatctctaactac

SEQ ID NO: 92 - DNA, 21 bp, Artificial Sequence
  Antibody fragment
agccaggacatctccaactac

SEQ ID NO: 93 - DNA, 9 bp, Artificial Sequence
  Antibody fragment
tacactagc

SEQ ID NO: 94 - DNA, 9 bp, Artificial Sequence
  Antibody fragment
tacacctcc

SEQ ID NO: 95 - DNA, 18 bp, Artificial Sequence
  Antibody fragment
tactataacctgccctgg

SEQ ID NO: 96 - DNA, 18 bp, Artificial Sequence
  Antibody fragment
tactacaacctgccctgg

SEQ ID NO: 97 - DNA, 15 bp, Artificial Sequence
  Antibody fragment
aactacgggatgaac

SEQ ID NO: 98 - DNA, 51 bp, Artificial Sequence
  Antibody fragment
tggattaacaccgacaccggcgagcctacctacgccgacgactttaagggc

SEQ ID NO: 99 - DNA, 48 bp, Artificial Sequence
  Antibody fragment
aaccccccctactactacggcactaacaacgccgaggctatggactac

SEQ ID NO: 100 - DNA, 21 bp, Artificial Sequence
  Antibody fragment
ggcttcaccctgactaactac

SEQ ID NO: 101 - protein, 447 aa, Artificial Sequence
  Antibody fragment
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 102 - protein, 214 aa, Artificial Sequence
  Antibody fragment
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 446 aa, Artificial Sequence
  Antibody fragment
QVQLKESGPGLVAPSQSLSITCTVSGFSLTAYGVNWVRQPPGKGLEWLGMIWDDGSTDYNSALKSRLSISKDNSKSQVFLKMNSLQTDDTARYYCAREGDVAFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 220 aa, Artificial Sequence
  Antibody fragment
DIVMTQSPSSLAVSVGQKVTMSCKSSQSLLNGSNQKNYLAWYQQKPGQSPKLLVYFASTRDSGVPDRFIGSGSGTDFTLTISSVQAEDLADYFCLQHFGTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 10 aa, Artificial Sequence
  Antibody fragment
GFTLTNYGMN

SEQ ID NO: 106 - protein, 5 aa, Artificial Sequence
  Antibody fragment
SYNMH

SEQ ID NO: 107 - protein, 17 aa, Artificial Sequence
  Antibody fragment
DIYPGNGDTSYNQKFKG

SEQ ID NO: 108 - protein, 9 aa, Artificial Sequence
  Antibody fragment
VGGAFPMDY

SEQ ID NO: 109 - protein, 7 aa, Artificial Sequence
  Antibody fragment
GYTFTSY

SEQ ID NO: 110 - protein, 6 aa, Artificial Sequence
  Antibody fragment
YPGNGD

SEQ ID NO: 111 - protein, 118 aa, Artificial Sequence
  Antibody fragment
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 112 - DNA, 354 bp, Artificial Sequence
  Antibody fragment
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagc

SEQ ID NO: 113 - protein, 444 aa, Artificial Sequence
  Antibody fragment
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 114 - DNA, 1332 bp, Artificial Sequence
  Antibody fragment
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 115 - protein, 15 aa, Artificial Sequence
  Antibody fragment
RASESVEYYGTSLMQ

SEQ ID NO: 116 - protein, 7 aa, Artificial Sequence
  Antibody fragment
AASNVES

SEQ ID NO: 117 - protein, 9 aa, Artificial Sequence
  Antibody fragment
QQSRKDPST

SEQ ID NO: 118 - protein, 11 aa, Artificial Sequence
  Antibody fragment
SESVEYYGTSL

SEQ ID NO: 119 - protein, 3 aa, Artificial Sequence
  Antibody fragment
AAS

SEQ ID NO: 120 - protein, 6 aa, Artificial Sequence
  Antibody fragment
SRKDPS

SEQ ID NO: 121 - protein, 111 aa, Artificial Sequence
  Antibody fragment
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIK

SEQ ID NO: 122 - DNA, 333 bp, Artificial Sequence
  Antibody fragment
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaag

SEQ ID NO: 123 - protein, 218 aa, Artificial Sequence
  Antibody fragment
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - DNA, 654 bp, Artificial Sequence
  Antibody fragment
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 125 - protein, 17 aa, Artificial Sequence
  Antibody fragment
DIYPGQGDTSYNQKFKG

SEQ ID NO: 126 - protein, 6 aa, Artificial Sequence
  Antibody fragment
YPGQGD

SEQ ID NO: 127 - protein, 118 aa, Artificial Sequence
  Antibody fragment
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSS

SEQ ID NO: 128 - DNA, 354 bp, Artificial Sequence
  Antibody fragment
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagc

SEQ ID NO: 129 - protein, 444 aa, Artificial Sequence
  Antibody fragment
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 130 - DNA, 1332 bp, Artificial Sequence
  Antibody fragment
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 131 - protein, 111 aa, Artificial Sequence
  Antibody fragment
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIK

SEQ ID NO: 132 - DNA, 333 bp, Artificial Sequence
  Antibody fragment
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaag

SEQ ID NO: 133 - protein, 218 aa, Artificial Sequence
  Antibody fragment
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - DNA, 654 bp, Artificial Sequence
  Antibody fragment
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 135 - protein, 114 aa, Artificial Sequence
  Antibody fragment
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 136 - protein, 108 aa, Artificial Sequence
  Antibody fragment
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 137 - protein, 120 aa, Artificial Sequence
  Antibody fragment
EVQVLESGGGLVQPGGSLRLYCVASGFTFSGSYAMSWVRQAPGKGLEWVSAISGSGGSTYYADSVKGRFTISRDNSKNTLYLQMNSLRAEDTAVYYCAKKYYVGPADYWGQGTLVTVSSG

SEQ ID NO: 138 - protein, 113 aa, Artificial Sequence
  Antibody fragment
DIVMTQSPDSLAVSLGERATINCKSSQSVLYSSNNKNYLAWYQHKPGQPPKLLIYWASTRESGVPDRFSGSGSGTDFTLTISSLQAEDVAVYYCQQYYSSPLTFGGGTKIEVK

SEQ ID NO: 139 - protein, 121 aa, Artificial Sequence
  Antibody fragment
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSS

SEQ ID NO: 140 - protein, 107 aa, Artificial Sequence
  Antibody fragment
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIK

SEQ ID NO: 141 - protein, 451 aa, Artificial Sequence
  Antibody fragment
EVQLVESGGGLVQSGGSLRLSCAASGFSLSSYGVDWVRQAPGKGLEWVGVIWGGGGTYYASSLMGRFTISRDNSKNTLYLQMNSLRAEDTAVYYCARHAYGHDGGFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 214 aa, Artificial Sequence
  Antibody fragment
EIVMTQSPATLSVSPGERATLSCRASESVSSNVAWYQQRPGQAPRLLIYGASNRATGIPARFSGSGSGTDFTLTISRLEPEDFAVYYCGQSYSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - DNA, 363 bp, Artificial Sequence
  Antibody fragment
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctcc

SEQ ID NO: 144 - DNA, 321 bp, Artificial Sequence
  Antibody fragment
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaag

SEQ ID NO: 145 - DNA, 1353 bp, Artificial Sequence
  Antibody fragment
gaggtgcagctggtggaatctggcggcggactggtgcagtccggcggctctctgagactgtcttgcgctgcctccggcttctccctgtcctcttacggcgtggactgggtgcgacaggcccctggcaagggcctggaatgggtgggagtgatctggggcggaggcggcacctactacgcctcttccctgatgggccggttcaccatctcccgggacaactccaagaacaccctgtacctgcagatgaactccctgcgggccgaggacaccgccgtgtactactgcgccagacacgcctacggccacgacggcggcttcgccatggattattggggccagggcaccctggtgacagtgtcctccgctagcaccaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 146 - DNA, 642 bp, Artificial Sequence
  Antibody fragment
gagatcgtgatgacccagtcccccgccaccctgtctgtgtctcccggcgagagagccaccctgagctgcagagcctccgagtccgtgtcctccaacgtggcctggtatcagcagagacctggtcaggcccctcggctgctgatctacggcgcctctaaccgggccaccggcatccctgccagattctccggctccggcagcggcaccgacttcaccctgaccatctcccggctggaacccgaggacttcgccgtgtactactgcggccagtcctactcataccccttcaccttcggccagggcaccaagctggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 147 - protein, 5 aa, Artificial Sequence
  Antibody fragment
SYGVD

SEQ ID NO: 148 - protein, 7 aa, Artificial Sequence
  Antibody fragment
GFSLSSY

SEQ ID NO: 149 - protein, 16 aa, Artificial Sequence
  Antibody fragment
VIWGGGGTYYASSLMG

SEQ ID NO: 150 - protein, 5 aa, Artificial Sequence
  Antibody fragment
WGGGG

SEQ ID NO: 151 - protein, 13 aa, Artificial Sequence
  Antibody fragment
HAYGHDGGFAMDY

SEQ ID NO: 152 - protein, 11 aa, Artificial Sequence
  Antibody fragment
RASESVSSNVA

SEQ ID NO: 153 - protein, 7 aa, Artificial Sequence
  Antibody fragment
SESVSSN

SEQ ID NO: 154 - protein, 7 aa, Artificial Sequence
  Antibody fragment
GASNRAT

SEQ ID NO: 155 - protein, 3 aa, Artificial Sequence
  Antibody fragment
GAS

SEQ ID NO: 156 - protein, 9 aa, Artificial Sequence
  Antibody fragment
GQSYSYPFT

SEQ ID NO: 157 - protein, 6 aa, Artificial Sequence
  Antibody fragment
SYSYPF

SEQ ID NO: 158 - protein, 124 aa, Artificial Sequence
  Antibody fragment
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 159 - protein, 107 aa, Artificial Sequence
  Antibody fragment
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 160 - protein, 447 aa, Artificial Sequence
  Antibody fragment
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 161 - protein, 215 aa, Artificial Sequence
  Antibody fragment
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 121 aa, Artificial Sequence
  Antibody fragment
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLWEVRALPSVYWGQGTLVTVSS

SEQ ID NO: 163 - protein, 109 aa, Artificial Sequence
  Antibody fragment
SYELTQPPSVSVAPGQTARITCGANDIGSKSVHWYQQKAGQAPVLVVSEDIIRPSGIPERISGSNSGNTATLTISRVEAGDEADYYCQVWDRDSDQYVFGTGTKVTVLG

SEQ ID NO: 164 - protein, 114 aa, Artificial Sequence
  Antibody fragment
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 165 - protein, 170 aa, Artificial Sequence
  Antibody fragment
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 166 - protein, 114 aa, Artificial Sequence
  Antibody fragment
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 167 - protein, 297 aa, Artificial Sequence
  Antibody fragment
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 114 aa, Artificial Sequence; identical to SEQ ID NO: 164
  Antibody fragment

SEQ ID NO: 169 - protein, 114 aa, Artificial Sequence
  Antibody fragment
NWVNVISDLKKIEDLIQSMHIDATLYTESDVHPSCKVTAMKCFLLELQVISLESGDASIHDTVENLIILANNSLSSNGNVTESGCKECEELEKKNIKEFLQSFVHIVQMFINTS

SEQ ID NO: 170 - protein, 77 aa, Artificial Sequence
  Antibody fragment
ITCPPPMSVEHADIWVKSYSLYSRERYICNSGFKRKAGTSSLTECVLNKATNVAHWTTPSLKCIRDPALVHQRPAPP

SEQ ID NO: 171 - protein, 22 aa, Artificial Sequence
  Synthetic peptide
YGRKKRRQRRRLYRSPAMPENL